(19) United States
(12) Patent Application Publication
(10) Pub. No.: US 2010/0063023 A1
(43) Pub. Date: Mar. 11, 2010

(54) Bicyclic and Tricyclic Substituted 6-Methylidene Carbapenems as Broad Spectrum Beta-Lactamase Inhibitors

(75) Inventors: Tarek Suhayl Mansour, New City, NY (US); Aranapakam Mudumbai Venkatesan, Rego Park, NY (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/555,850

(22) Filed: Sep. 9, 2009

Related U.S. Application Data
(60) Provisional application No. 61/095,649, filed on Sep. 10, 2008.

(51) Int. Cl. A61K 31/407; C07D 487/04; A61P 31/04; A61K 31/546

(52) U.S. Cl. 514/210.09; 540/302; 514/210.05

(57) ABSTRACT

Provided is a β-lactamase antibiotic and a compound of formula I, a process of producing the compound, pharmaceutical compositions and the use thereof for the treatment of bacterial infection or disease in a patient in need thereof.

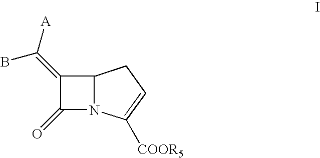

### FIELD OF INVENTION

In one aspect, these teachings relate to bicyclic and tricyclic 6-alkylidene penems, which act as broad-spectrum β-lactamase inhibitors. In another aspect, the compounds of the present invention can be combined with a β-lactamase antibiotic, including a “fourth-generation” cephalosporin antibiotic such as, for example, cefepime, a penicillin antibiotic, or a carbapenem antibiotic. β-lactamases hydrolyze β-lactamase antibiotics, and as such serve as the primary cause of bacterial resistance. The compounds of the present teachings when combined with a β-lactamase antibiotic such as, cefepime provide an effective treatment against life threatening bacterial infections.

### BACKGROUND

Penicillin and cephalosporin are the most frequently and widely used β-lactamase antibiotics in the clinic. However, the development of resistance to β-lactamase antibiotics by different pathogens has had a damaging effect on maintaining the effective treatment of bacterial infections. (Coleman, K. Expert Opin. Invest. Drugs 1995, 4, 693; Sutherland, R. Infection 1995, 23 (4) 191; Bush, K, Cur. Pharm. Design 1999, 5, 839-845). The most significant known mechanism related to the development of bacterial resistance to the β-lactamase antibiotics is the production of Class-A, Class-B and Class-C serine β-lactamase. These enzymes degrade the β-lactamase antibiotics, resulting in the loss of antibacterial activity. Class-A enzymes preferentially hydrolyze penicillin where as Class-C lactamase has a substrate profile favoring cephalosporin hydrolysis. (Bush, K.; Jacoby, G. A.; Medeiros, A. A. Antimicrob. Agents Chemother. 1995, 39, 1211). To date over 250 different β-lactamase have been reported (Payne, D. J., Du, W and Bateson, J. H. Exp. Opin. Invest. Drugs 2000, 247) and there is a need for a new generation of broad-spectrum β-lactamase inhibitors. Bacterial resistance to these antibiotics can be greatly reduced by administering the β-lactamase antibiotic in combination with a compound, which inhibits these enzymes.

Cefepime is a parenteral aminothiazolylacetamido cephalosporin antibiotic. (Sanders, C. C. 1993. Cefepime: the next generation? Clin. Infect. Dis. 17:369-379). Even though cefepime was shown to have good activity against many pathogens that cause nosocomial pneumonia and other serious infections, it is not active against Enterococcus faecalis, Clostridium difficile and methicillin-resistant S. aureus. (Jand ones, R. N. 2001. Resistance patterns among nosocomial pathogens: trends over the past few years. Chest 119:397S-404S; Okamoto, M. P., R. K. Nakahiro, A. Chin, A. Bedikian, and M. V. Gill. 1994. Cefepime: a new fourth-generation cephalosporin. Am. J. Hosp. Pharm. 41:463-477.) Cefepime is also hydrolyzed by the extended-spectrum beta-lactamases (ESBLs) produced by some members of the Enterobacteriaceae.

Commercially available β-lactamase inhibitors such as, for example, clavulanic acid, sulbactam and tazobactam are all effective against Class-A producing pathogens. Clavulanic acid is clinically used in combination with amoxicillin and ticarcillin; similarly sulbactam with ampicillin and tazobactam with piperacillin. However, these compounds are ineffective against Class-C producing organisms. The mechanism of inactivation of Class-A, β-lactamases (such as, for example, PCI and TEM-1) has been elucidated. (Bush, K.; Antimicrob. Agents Chemother. 1993, 37, 851; Yang, Y.; Janota, K.; Tabei, K.; Huang, N.; Seigal, M. M.; Lin, Y. I.; Rasmussen, B. A. and Shlaes, D. M. J. Biol. Chem. 2000, 35, 26674-26682).

Recently it has been shown that 6-methylidene derivatives of general formula (II) are effective, broad-spectrum β-lactamase inhibitors when combined with β-lactamase antibiotics. WO 03/093280 A1, WO 03/093279 A1, WO 03/093277 A1, and US 2004 132708 A1 all of which are incorporated herein by reference in their entirety.

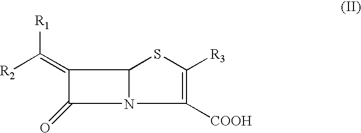

However, there remains a need for effective treatments against life threatening bacterial infections. The present invention is directed to these and other important ends.

### SUMMARY

In one aspect, there is provided compounds of formula I:

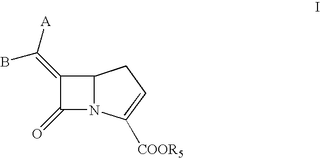

wherein the constituent variables are as defined herein.

In another aspect, there is provided pharmaceutical compositions containing a compound of the invention, and a pharmaceutically acceptable carrier.

In further aspects, there is provided methods for the treatment of a patient suffering from a chronic condition such as, schizophrenia, paranoia, manic-depressive illness, or anxiety.

In yet other aspects, there is provided methods for producing compounds of Formula I. Other aspects of the present invention are described further in the following detailed description.

### DETAILED DESCRIPTION

The present teachings relate to low molecular weight, broad-spectrum compounds having 6-lactamase inhibitory and antibacterial properties. The present teachings further relate to a class of bicyclic and tricyclic 6-alkylidine penems, salts or derivatives thereof in which the compounds are useful in the treatment of antibacterial infections in humans or animals, when used alone or in combination with other antibiotics.

In accordance with the present teachings there are provided compounds of general formula I or a pharmaceutically acceptable salt or in vivo hydrolyzable ester thereof:

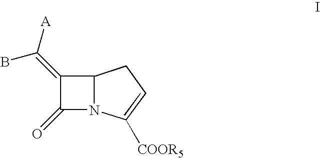

wherein:

one of A and B denotes hydrogen and the other an 8- to 14-membered fused bicyclic or tricyclic heteroaryl group optionally substituted with nitro, -aryl, -heteroaryl, C₁₋₆alkoxycarbonyl-, C₁₋₆alkoxy, -alkoxyalkyl, alkyl-O—C₂₋₄alkyl-O—, -cyano, -halogen, -hydroxy, —N—R₆R₇, -trifluoromethyl, -trifluoromethoxy, arylalkyl, alkylaryl, R₆R₇N-alkyl-, HO—O₁₋₆alkyl-, alkoxyalkyl-, alkyl-S—, —SO₂N—R₆R₇, —SO₂NHR₆, —CO₂H, CONR₆R₇, aryl-O—, heteroaryl-O—, —S(═O)ₛaryl where s is 0-2, -alkyl-O-alkyl-NR₆R₇, -alkyl-aryl-O-alkylN—R₆R₇, C₂₋₈alkenyl, C₂₋₆alkynyl, C₃₋₇cycloalkyl, alkoxy-alkyl-O—, R₆R₇N—(C₁₋₆alkyl), and —S(═O)ₛ-heteroaryl where s is 0-2;

R₅ is H, C₁₋₆alkyl, C₅₋₆cycloalkyl, or CHR₃OCOC₁₋₆alkyl; and

R₃ is hydrogen, C₁₋₆alkyl, C₃₋₇cycloalkyl, C₆₋₁₄aryl, or 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, wherein aryl or heteroaryl can be optionally substituted with 1 or 2 of nitro, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl, C₁₋₆alkoxy, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, alkyl-S—, —SO₂N—R₆R₇, aryl-O—, heteroaryl-O—, —S((═O)ₛ-aryl where s is 0-2, -alkyl-O-alkyl-NR₆R₇, -alkyl-aryl-O-alkylN—R₆R₇, -alkyl-aryl-O-alkylN—R₅R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; or a pharmaceutically acceptable salt thereof.

Fused heteroaryl groups of the present teachings include both fused bicyclic and fused tricyclic heteroaryl groups.

Some examples of fused bicyclic heteroaryl groups are as follows:

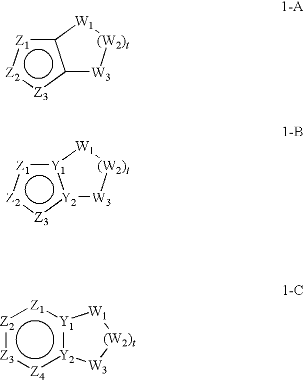

Further examples of bicyclic heteroaryl groups are as follows:

In an Embodiment of Formula 1-A:

Z₁, Z₂ and Z₃ are independently CR₂, N, O, S, or N—R₁; and any one of Z₁, Z₂, and Z₃ is a carbon bonded to the remainder of the molecule as shown in formula I; or

one of Z₁ or Z₂ is CR₂; the other Z₁ or Z₂ and Z₃ independently can be N, O, S, or N—R₁, in any combination consistent with the aromaticity of the ring; or

two of Z₁, Z₂, and Z₃ are CR₂; the other can be optionally selected from N, O, S, and N—R₁ in any combination consistent with the aromaticity of the ring;

W₁, W₂, and W₃ are each independently CR₄R₄, S, SO, SO₂, O, N—R₁, or C(═O); with the proviso that they form no S—S or O—O or S—O bond; and

t is 1-4;

R₁ is H, —CONR₆R₇, —SO₂NR₆R₇, or is a group selected from C₁₋₆alkyl, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₃₋₇cycloalkyl, C₂₋₈alkenyl, C₂₋₆alkynyl, C₁₋₆perfluoroalkyl, —S(═O)₂C₁₋₆alkyl or C₆₋₁₄aryl, —C(═O)heteroaryl, —C(═O)aryl, —C(═O)(C₁₋₆alkyl), —C(═O)C₃₋₆cycloalkyl, —C(═O)mono or bicyclic saturated heterocyclyl, C₁₋₆alkylaryl, (C₁₋₆alkyl)heteroaryl, aryl(C₁₋₆alkyl), heteroaryl(C₁₋₆alkyl), C₁₋₆alkyl mono or bicyclic saturated heterocyclyl, arylalkenyl of 8 to 16 carbon atoms, arylalkyloxyalkyl, (C₁₋₆alkyl)O(C₁₋₆alkyl)-aryl, (C₁₋₆alkyl)O(C₁₋₆alkyl)heteroaryl, aryloxyalkyl, heteroaryloxyalkyl, aryloxyaryl, aryloxyheteroaryl, alkylaryloxyaryl, alkylaryloxyheteroaryl, alkylaryloxyalkylamines, C₁₋₆alkoxycarbonyl, aryloxy carbonyl, heteroaryloxy carbonyl, said group being optionally substituted with nitro, -aryl, heteroaryl, C₁₋₆alkoxycarbonyl, C₁₋₆alkoxy, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, alkyl-S—, —SO₂N—R₆R₇, aryl-O—, heteroaryl-O—, —S((═O)ₛ-aryl where s is 0-2, -alkyl-O-alkyl—NR₆R₇, -alkyl-aryl-O-alkylN—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; provided that the atom of R₁ which bonds to N is not a carbon that is double or triple bonded to another carbon;

R₂ is absent, hydrogen, halogen, cyano, N—R₆R₇, hydroxy; COOR₆, C₁₋₆alkylamino (C₁₋₆alkoxy), alkylenedioxy, C₁₋₆perfluoroalkyl, CONR₆R₇, guanidino or cyclic guanidino, SO₂NR₆R₇, C₁₋₆alkyl, C₂₋₈alkenyl having 1 to 2 double bonds, C₂₋₆alkynyl having 1 to 2 triple bonds; C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxy, alkylaryloxy alkylamines, aryloxy, heteroaryloxy, alkenyloxy, alkynyloxy, aryloxyalkyl amine, C₁₋₆perfluoroalkyl, S(═O)_{q}—C₁₋₆alkyl, S(═O)_{q}— where q is 0-2, alkylaryl, arylalkyl, C₁₋₆alkylheteroaryl, heteroaryl-C₁₋₆alkyl, C₁₋₆alkyl mono or bicyclic saturated heterocyclyl having 1-3 heteroatoms independently selected from O, N or S, arylalkenyl of 8 to 16 carbon atoms, arylalkyloxyalkyl, aryloxyalkyl, heteroaryloxyalkyl, aryloxyaryl, aryloxyheteroaryl, heteroaryloxyaryl, alkyl aryloxyaryl, alkylaryloxyheteroaryl, aryloxyalkyl, heteroaryloxyalkyl, alkylaryloxyalkylamines, C₃₋₇cycloalkyl, saturated or partially saturated heterocyclyl, said group being optionally substituted with nitro, C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, C₁₋₆alkyl-S—, —SO₂N—R₆R₇, C₆₋₁₄aryl-O—, heteroaryl-O—, —S((═O)ₛ-aryl where s is 0-2, -alkyl-O-alkyl-NR₆R₇, -alkyl-aryl-O-alkylN—R₆R₇, -alkyl-aryl-O-alkylN—R₅R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; provided that the atom of R₂ that bonds to N is not a carbon that is double or triple bonded to another carbon;

each R₄ is independently H or C₁₋₆alkyl optionally substituted with nitro, C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl, C₁₋₆alkoxy, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, C₁₋₆alkyl-S—, —SO₂N—R₆R₇, C₆₋₁₄aryl-O—, heteroaryl-O—, —S((═O)ₛ-aryl where s is 0-2, -alkyl-O-alkyl-NR₆R₇, -alkyl-aryl-O-alkylN—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; or any one of R₄ can be OH, C₁₋₆alkoxy, —S—C₁₋₆alkyl, COOR₆, —NR₆R₇, —CONR₆R₇; or R₄R₄ may together be (═O); or R₄R₄ together with the carbon to which they are attached may form a spiro ring of five to eight members optionally having 1-3 heteroatoms selected from N, O, S(═O)ₙ where n is 0-2, and N—R₁; and

R₆ and R₇ are independently H, or a group selected from C₁₋₆alkyl, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, alkylaryl, arylalkyl, heteroarylalkyl, C₁₋₆alkylheteroaryl, said group being optionally substituted with nitro, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl-, C₁₋₆alkoxy, -alkoxyalkyl, alkyl-O—C₂₋₄alkyl -O-cyano, -halogen, -hydroxy, —NR₆R₇, —COOH, —COO-alkyl, -trifluoromethyl, -trifluoromethoxy, arylalkyl, alkylaryl, R₆R₇N-alkyl-, HO—C₁₋₆alkyl, alkoxyalkyl-, C₁₋₆alkyl-S—, —SO₂NHR₆, —CO₂H, CONR₆R₇, C₆₋₁₄aryl-O—, heteroaryl-O—, —S(═O)ₛ-aryl, wherein s is 0-2, -alkyl-O-alkyl-NR₆R₇, -alkyl-aryl-O-alkyl-N—R₆R_{7,} C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl, wherein S is 0-2, or R₆ and R₇ can together with the nitrogen to which they are attached form a 3 to 7 membered saturated ring system optionally having one or two heteroatoms selected from N—R₁, O, and S(═O)ₙ, where n is 0-2.

In an Embodiment of Formula 1-B:

two of Z₁, Z₂, and Z₃ are independently CR₂, N, O, S, or N—R₁ and the other is a carbon bonded to the remainder of the molecule; or

one of Z₁, Z₂, or Z₃ is CR₂ and the other two are independently CR₂, N, O, S, or N—R₁ in any combinations consistent with the aromaticity of the ring; or

two of Z₁, Z₂, and Z₃ are N and the other is carbon bonded to the penem portion of the molecule; W₁, W₂, and W₃ are independently CR₄R₄, S, SO, SO₂, O, or N—R₁; Y₁, and Y₂ are N or C;

t is1-4;

R₁ is H, —CONR₆R₇, —SO₂NR₆R₇, or is a group selected from C₁₋₆alkyl, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₃₋₇cycloalkyl, C₂₋₈alkenyl, C₂₋₆alkynyl, C₁₋₆perfluoroalkyl, —S(═O)₂C₁₋₆alkyl or C₆₋₁₄aryl, —C(═O)heteroaryl, —C(═O)aryl, —C(═O)(C₁₋₆alkyl), —C(═O)C₃₋₆cycloalkyl, —C(═O)mono or bicyclic saturated heterocyclyl, C₁₋₆alkylaryl, (C₁₋₆alkyl)heteroaryl, aryl(C₁₋₆alkyl), heteroaryl(C₁₋₆alkyl), C₁₋₆alkyl mono or bicyclic saturated heterocyclyl, arylalkenyl of 8 to 16 carbon atoms, arylalkyloxyalkyl, (C₁₋₆alkyl)O(C₁₋₆alkyl)-aryl, (C₁₋₆alkyl)O(C₁₋₆alkyl)heteroaryl, aryloxyalkyl, heteroaryloxyalkyl, aryloxyaryl, aryloxyheteroaryl, alkylaryloxyaryl, alkylaryloxyheteroaryl, alkylaryloxyalkylamines, C₁₋₆alkoxycarbonyl, aryloxy carbonyl, heteroaryloxy carbonyl, said group being optionally substituted with nitro, -aryl, heteroaryl, C₁₋₆alkoxycarbonyl, C₁₋₆alkoxy, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, alkyl-S—, —SO₂N—R₆R₇, aryl-O—, heteroaryl-O—, —S((═O)ₛ-aryl where s is 0-2, -alkyl-O-alkyl-NR₆R₇, -alkyl-aryl-O-alkylN—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; provided the atom of R₁ which bonds to N is not a carbon that is double or triple bonded to another carbon;

R₂ is absent, hydrogen, halogen, cyano, N—R₆R₇, hydroxy; COOR₆, C₁₋₆alkylamino (C₁₋₆alkoxy), alkylenedioxy, C₁₋₆perfluoroalkyl, CONR₆R₇, guanidino or cyclic guanidino, SO₂NR₆R₇, C₁₋₆alkyl, C₂₋₈alkenyl having 1 to 2 double bonds, C₂₋₆alkynyl having 1 to 2 triple bonds; C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxy, alkylaryloxy alkylamines, aryloxy, heteroaryloxy, alkenyloxy, alkynyloxy, aryloxyalkyl amine, C₁₋₆perfluoroalkyl, S(═O)_{q}—C₁₋₆alkyl, S(═O)_{q}— where q is 0-2, alkylaryl, arylalkyl, C₁₋₆alkylheteroaryl, heteroaryl-C₁₋₆alkyl, C₁₋₆alkyl mono or bicyclic saturated heterocyclyl having 1-3 heteroatoms independently selected from O, N or S, arylalkenyl of 8 to 16 carbon atoms, arylalkyloxyalkyl, aryloxyalkyl, heteroaryloxyalkyl, aryloxyaryl, aryloxyheteroaryl, heteroaryloxyaryl, alkyl aryloxyaryl, alkylaryloxyheteroaryl, aryloxyalkyl, heteroaryloxyalkyl, alkylaryloxyalkylamines, C₃₋₇cycloalkyl, saturated or partially saturated heterocyclyl, said group being optionally substituted with nitro, C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl, C₁₋₆alkoxy, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, C₁₋₆alkyl-S—, —SO₂N—R₆R₇, C₆₋₁₄aryl-O—, heteroaryl-O—, —S((═O)ₛ-aryl where s is 0-2, -alkyl-O-alkyl-NR₆R₇, -alkyl-aryl-O-alkylN—R₆R₇, -alkyl-aryl-O-alkylN—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; provided that the atom of R₂ that bonds to N is not a carbon that is double or triple bonded to another carbon;

each R₄ is independently H or C₁₋₆alkyl optionally substituted with nitro, C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl, C₁₋₆alkoxy, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, C₁₋₆alkyl-S—, —SO₂N—R₆R₇, C₆₋₁₄aryl-O—, heteroaryl-O—, —S((═O)ₛ-aryl where s is 0-2, -alkyl-O-alkyl-NR₆R₇, -alkyl-aryl-O-alkylN—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; or any one of R₄ can be OH, C₁₋₆alkoxy, —S—C₁₋₆alkyl, COOR₆, —NR₆R₇, —CONR₆R₇; or R₄R₄ may together be (═O); or R₄R₄ together with the carbon to which they are attached may form a spiro ring of five to eight members optionally having 1-3 heteroatoms selected from N, O, S(═O)ₙ where n is 0-2, and N—R₁; and

R₆ and R₇ are independently H, or a group selected from C₁₋₆alkyl, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, alkylaryl, arylalkyl, heteroarylalkyl, C₁₋₆alkylheteroaryl, said group being optionally substituted with nitro, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl-, C₁₋₆alkoxy, -alkoxyalkyl, alkyl-O—C₂₋₄alkyl-O-cyano, -halogen, -hydroxy, —NR₆R₇, —COOH, —COO-alkyl, -trifluoromethyl, -trifluoromethoxy, arylalkyl, alkylaryl, R₆R₇N-alkyl-, HO—C₁₋₆alkyl, alkoxyalkyl-, C₁₋₆alkyl-S—, —SO₂NHR₆, —CO₂H, CONR₆R₇, C₆₋₁₄aryl-O—, heteroaryl-O—, —S(═O)ₛ-aryl, wherein s is 0-2, -alkyl-O-alkyl-NR₆R₇, -alkyl-aryl-O-alkyl-N—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl, wherein S is 0-2, or R₆ and R₇ can together with the nitrogen to which they are attached form a 3 to 7 membered saturated ring system optionally having one or two heteroatoms selected from N—R₁, O, and S(═O)ₙ, where n is 0-2.

In an Embodiment of the Formula 1-C:

three of Z₁, Z₂, Z₃, and Z₄ are independently CR₂ or N; and one of Z₁, Z₂, Z₃, and Z₄ is a carbon bonded to the remainder of the molecule;

W₁, W₂, and W₃ are independently CR₄R₄, S, SO, SO₂, O, or N—R₁; with the proviso that they form no S—S or O—O or S—O bond; Y₁ and Y₂ are independently C or N;

t is 1 to 4;

R₁ is H, —CONR₆R₇, —SO₂NR₆R₇, or is a group selected from C₁₋₆alkyl, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or 5, C₃₋₇cycloalkyl, C₂₋₈alkenyl, C₂₋₆alkynyl, C₁₋₆perfluoroalkyl, —S(═O)₂C₁₋₆alkyl or C₆₋₁₄aryl, —C(═O)heteroaryl, —C(═O)aryl, —C(═O)(C₁₋₆alkyl), —C(═O)C₃₋₆cycloalkyl, —C(═O)mono or bicyclic saturated heterocyclyl, C₁₋₆alkylaryl, (C₁₋₆alkyl)heteroaryl, aryl(C₁₋₆alkyl), heteroaryl(C₁₋₆alkyl), C₁₋₆alkyl mono or bicyclic saturated heterocyclyl, arylalkenyl of 8 to 16 carbon atoms, arylalkyloxyalkyl, (C₁₋₆alkyl)O(C₁₋₆alkyl)-aryl, (C₁₋₆alkyl)O(C₁₋₆alkyl)heteroaryl, aryloxyalkyl, heteroaryloxyalkyl, aryloxyaryl, aryloxyheteroaryl, alkylaryloxyaryl, alkylaryloxyheteroaryl, alkylaryloxyalkylamines, C₁₋₆alkoxycarbonyl, aryloxy carbonyl, heteroaryloxy carbonyl, said group being optionally substituted with nitro, -aryl, heteroaryl, C₁₋₆alkoxycarbonyl, C₁₋₆alkoxy, -alkoxyalkyl, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, alkyl-S—, —SO₂N—R₆R₇, aryl-O—, heteroaryl-O—, —S((═O)ₛ-aryl where s is 0-2, -alkyl-O-alkyl-NR₆R₇, -alkyl-aryl-O-alkylN—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; provided the atom of R₁ which bonds to N is not a carbon that is double or triple bonded to another carbon;

R₂ is absent, hydrogen, halogen, cyano, N—R₆R₇, hydroxy; COOR₆, C₁₋₆alkylamino (C₁₋₆alkoxy), alkylenedioxy, C₁₋₆perfluoroalkyl, CONR₆R₇, guanidino or cyclic guanidino, SO₂NR₆R₇, C₁₋₆alkyl, C₂₋₈alkenyl having 1 to 2 double bonds, C₂₋₆alkynyl having 1 to 2 triple bonds; C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxy, alkylaryloxy alkylamines, aryloxy, heteroaryloxy, alkenyloxy, alkynyloxy, aryloxyalkyl amine, C₁₋₆perfluoroalkyl, S(═O)_{q}—C₁₋₆alkyl, S(═O)_{q}— where q is 0-2, alkylaryl, arylalkyl, C₁₋₆alkylheteroaryl, heteroaryl-C₁₋₆alkyl, C₁₋₆alkyl mono or bicyclic saturated heterocyclyl having 1-3 heteroatoms independently selected from O, N or S, arylalkenyl of 8 to 16 carbon atoms, arylalkyloxyalkyl, aryloxyalkyl, heteroaryloxyalkyl, aryloxyaryl, aryloxyheteroaryl, heteroaryloxyaryl, alkyl aryloxyaryl, alkylaryloxyheteroaryl, aryloxyalkyl, heteroaryloxyalkyl, alkylaryloxyalkylamines, C₃₋₇cycloalkyl, saturated or partially saturated heterocyclyl, said group being optionally substituted with nitro, C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl, C₁₋₆alkoxy, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, C₁₋₆alkyl-S—, —SO₂N—R₆R₇, C₆₋₁₄aryl-O-, heteroaryl-O—, —S((═O)ₛ-aryl where s is 0-2, -alkyl-O-alkyl-NR₆R₇, -alkyl-aryl-O-alkylN—R₆R₇, -alkyl-aryl-O-alkylN—R₅R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; provided that the atom of R₂ that bonds to N is not a carbon that is double or triple bonded to another carbon;

each R₄ is independently H or C₁₋₆alkyl optionally substituted with nitro, C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl, C₁₋₆alkoxy, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, C₁₋₆alkyl-S—, —SO₂N—R₆R₇, C₆₋₁₄aryl-O—, heteroaryl-O—, —S((═O)ₛ-aryl where s is 0-2, -alkyl-O-alkyl-NR₆R₇, -alkyl-aryl-O-alkylN—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; or any one of R₄ can be OH, C₁₋₆alkoxy, —S—C₁₋₆alkyl, COOR₆, —NR₆R₇, —CONR₆R₇; or R₄R₄ may together be (═O); or R₄R₄ together with the carbon to which they are attached may form a spiro ring of five to eight members optionally having 1-3 heteroatoms selected from N, O, S(═O)ₙ where n is 0-2, and N—R₁; and

R₆ and R₇ are independently H, or a group selected from C₁₋₆alkyl, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, alkylaryl, arylalkyl, heteroarylalkyl, C₁₋₆alkylheteroaryl, said group being optionally substituted with nitro, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl-, C₁₋₆alkoxy, -alkoxyalkyl, alkyl-O—C₂₋₄alkyl-O-cyano, -halogen, -hydroxy, —NR₆R₇, —COOH, —COO-alkyl, -trifluoromethyl, -trifluoromethoxy, arylalkyl, alkylaryl, R₆R₇N-alkyl-, HO—C₁₋₆alkyl, alkoxyalkyl-, C₁₋₆alkyl-S—, —SO₂NHR₆, —CO₂H, CONR₆R₇, C₆₋₁₄aryl-O—, heteroaryl-O—, —S(═O)ₛ-aryl, wherein s is 0-2, -alkyl-O-alkyl-NR₆R₇, -alkyl-aryl-O-alkyl-N—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl, wherein S is 0-2, or R₆ and R₇ can together with the nitrogen to which they are attached form a 3 to 7 membered saturated ring system optionally having one or two heteroatoms selected from N—R₁, O, and S(═O)ₙ, where n is 0-2.

In Some Embodiments of the Formula 1-A:

Z₁ is N, S, N—R₁, or O; one of Z₂ or Z₃ is CR₂; and the other of Z₂ or Z₃ is a carbon bonded to the remainder of the molecule; and t is 1-3; or

Z₃ is N, S, N—R₁, or O; one of Z₁ or Z₂ is CR₂; and the other of Z₂ or Z₁ is a carbon bonded to the remainder of the molecule; and t is 1-3; or

Z₂ is N, S, N—R₁, or O; one of Z₁ or Z₃ is CR₂; the other of Z₁ or Z₃ is a carbon bonded to the remainder of the molecule; and t is 1-3; or

Z₁ is N, N—R₁, O, or S; Z₂ is N, O, or S; Z₃ is a carbon bonded to the penem portion of the molecule; and t is 1-3; or

Z₃ is N, N—R₁, O, or S; Z₂ is N, O, or S; Z₁ is a carbon bonded to the penem portion of the molecule; and t is 1-3; or

Z₁ is N, N—R₁, O, or S; Z₃ is N, O, or S; Z₂ is a carbon bonded to the penem portion of the molecule; and t is 1-3; or

Z₁ is N, S, N—R₁, or O; Z₂ or Z₃ is CR₂; the other Z is a carbon bonded to the remainder of the molecule; W₁, W₂, and W₃ are independently CR₄R₄; and t is 1-3; or

Z₃ is N, S, N—R₁, or O; one of Z₂ or Z₁ is CR₂; the other Z is a carbon bonded to the remainder of the molecule; W₁, W₂, and W₃ are independently CR₄R₄; and t is 1-3; or

Z₂ is N, S, N—R₁ or O; one of Z₁ or Z₃ is CR₂; the other Z is a carbon bonded to the remainder of the molecule; W₁, W₂, and W₃ are independently CR₄R₄; and t is 1-3; or

Z₁ is N, N—R₁, O, or S; Z₂ is N, O, or S; Z₃ is a carbon bonded to the penem portion of the molecule; W₁, W₂, W₃ are independently CR₄R₄; and t is 1-3; or

Z₃ is N, N—R₁, O, or S; Z₂ is N, O, or S; Z₁ is a carbon bonded to the penem portion of the molecule; W₁, W₂, W₃ are independently CR₄R₄; and t is 1-3; or

Z₁ is N, N—R₁, O, or S; Z₃ is N, O, or S; Z₂ is a carbon bonded to the penem portion of the molecule; W₁, W₂, W₃ are independently CR₄R₄; and t is 1-3; or

Z₃ is N, N—R₁, O, or S; Z₁ is N, O, or S; Z₂ is a carbon bonded to the penem portion of the molecule; W₁, W₂, W₃ are independently CR₄R₄; and t is 1-3; or

Z₁ is N, S, N—R₁, or O; one of Z₂ or Z₃ is CR₂; the other Z is a carbon bonded to the remainder of the molecule; one W₂ is N—R₁, O, or S(═O)ₙ where n is 0-2; another W₂ is CR₄R₄; and t is 1-3; or

Z₃ is N, S, N—R₁, or O; one of Z₂ or Z₁ is CR₂; the other Z is a carbon bonded to the remainder of the molecule; one W₂ is N—R₁, O, or S(═O)ₙ where n is 0-2; another W₂ is CR₄R₄; and t is 1-3; or

Z₂ is N, S, N—R₁, or O; one of Z₁ or Z₃ is CR₂; the other Z is a carbon bonded to the remainder of the molecule; one W₂ is N—R₁, O, or S(═O)ₙ where n is 0-2; another W₂ is CR₄R₄; and t is 1-3; or

Z₁ is N, N—R₁, O, or S; Z₂ is N, O, or S; Z₃ is a carbon bonded to the penem portion of the molecule; one W₂ is N—R₁, O, or S(═O)ₙ where n is 0-2; another W₂ is CR₄R₄; and t is 1-3; or

Z₃ is N, N—R₁, O, or S; Z₂ is N, O, or S; Z₁ is a carbon bonded to the penem portion of the molecule; one W₂ is N—R₁, O, or S(═O)ₙ where n is 0-2; another W₂ is CR₄R₄; and t is 1-3; or

Z₁ is N, N—R₁, O, or S; Z₃ is N, O, or S; Z₂ is a carbon bonded to the penem portion of the molecule; one W₂ is N—R₁, O, or S(═O)ₙ where n is 0-2; another W₂ is CR₄R₄; and t is 1-3; or

Z₁ is N, S, N—R₁, or O; Z₂ or Z₃ is CR₂; and the other Z is a carbon bonded to the remainder of the molecule; W₁ and W₃ are CH₂ or both hydrogens on the methylene linkage can be substituted to form a spiro system with or without the presence of hetero atoms selected from O, S(═O)ₙ where n is 0-2, or N—R₁ to form a five- to eight-membered cyclic system; one W₂ is N—R₁, O or S(═O)ₙ where n is 0-2; another W₂ is CR₄R₄; and t is 1-3; or

Z₃ is N, S, N—R₁, or O; Z₂ or Z₁ is CR₂; the other Z is a carbon bonded to the remainder of the molecule; W₁ and W₃ are CR₄R₄; one W₂ is N—R₁, O, or S(═O)ₙ where n is 0-2; another W₂ is CR₄R₄; and t is 1-3; or

Z₂ is N, S, N—R₁, or O; Z₁ or Z₃ is CR₂; the other Z is carbon bonded to the remainder of the molecule; W₁ and W₃ are CR₄R₄; one W₂ is N—R₁, O, or S(═O)ₙ where n is 0-2; another W₂ is CR₄R₄; and t is 1-3; or

Z₁ is N, N—R₁, O, or S; Z₂ is N, O, or S; Z₃ is a carbon bonded to the penem portion of the molecule; W₁ and W₃ are CR₄R₄; one W₂ is N—R₁, O, or S(═O)ₙ where n is 0-2, another W₂ is CR₄R₄; and t is 1-3; or

Z₃ is N, N—R₁, O, or S; Z₂ is N, O, or S; Z₁ is a carbon bonded to the penem portion of the molecule; W₁ and W₃ are CR₄R₄; one W₂ is N—R₁, O, or S(═O)ₙ where n is 0-2; another W₂ is CR₄R₄; and t is 1-3; or

Z₁ is N, N—R₁, O, or S; Z₃ is N, O, or S; Z₂ is a carbon bonded to the penem portion of the molecule; W₁ and W₃ are CR₄R₄; one W₂ is N—R₁, O, or S(═O)ₙ where n is 0-2; another W₂ is CR₄R₄; and t is 1-3; or

Z₃ is N, N—R₁, O or S; Z₁ is N, O or S; Z₂ is a carbon bonded to the remainder of the molecule; W₁ and W₃ are CR₄R₄; one W₂ is N—R₁, O or S(═O)ₙ where n is 0-2; another W₂ is CR₄R_{4;} t is 1-3; and

R₁, R₂, R₄, R₆, and R₇ are as defined above.

In Some Embodiments of the Formula 1-B:

t is 3; and

Z₁ and Z₃ are N; Y₁ is N; Y₂ is C; and Z₂ is a carbon bonded to the remainder of the molecule; or

Z₂ and Z₃ are N; Y₁ is N; Y₂ is C; and Z₁ is a carbon bonded to the remainder of the molecule; or

Z₁ is N; Y₁ is N; Y₂ is C; one of Z₂ or Z₃ is CR₂; and the other Z is a carbon bonded to the remainder of the molecule; or

Z₁ is N; Y₁ is C; Y₂ is N; one of Z₂ or Z₃ is CR₂; and the other Z is a carbon bonded to the remainder of the molecule; or

Z₁ is N; Y₁ is N; Y₂ is C; one of Z₂ or Z₃ is CR₂; the other Z is a carbon bonded to the remainder of the molecule; W₁ and W₃ are independently CR₄R₄; one W₂ is N—R₁, O, S(═O)ₙ where n is 0-2; another W₂ is CR₄R₄; and t is 1-3; or

Z₁ is N; Y₁ is C; Y₂ is N; and one of Z₂ or Z₃ is CR₂ and the other Z is a carbon bonded to the remainder of the molecule; W₁ and W₃ are independently CR₄R₄; W₂ is N—R₁, O, S(═O)ᵣ where r is 0-2, and another W₂ is CR₄R₄; or

Z₃ is N; Y₁ is N; Y₂ is C; and one of Z₁ or Z₂ is CR₂ and the other is a carbon bonded to the remainder of the molecule; or

Z₂ is N; Y₁ is N; Y₂ is C; and one of Z₁ or Z₃ is CR₂ and the other is a carbon bonded to the remainder of the molecule; or

Z₁ and Z₂ are N; Y₁ is N; Y₂ is C; and Z₃ is carbon bonded to the remainder of the molecule; or

Z₁, Z₂ and Z₃ are independently CR₂; Y₁ is C; Y₂ is N; and any one of Z₁, Z₂, and Z₃ is a carbon bonded to the remainder of the molecule; or

Z₁ and Z₃ are N; Y₁ is N; Y₂ is C; Z₂ is a carbon bonded to the remainder of the molecule; and t is 1-3; or

Z₂ and Z₃ are N; Y₁ is N; Y₂ is C; and Z₁ is a carbon bonded to the remainder of the molecule; and t is 1-3; or

Z₂ and Z₃ are N; Y₁ is C; Y₂ is N; Z₁ is a carbon bonded to the remainder of the molecule; and t is 1-3; or

Z₂ and Z₃ are N; Y₁ is N; Y₂ is C; Z₁ is a carbon bonded to the remainder of the molecule; W₁ and W₃ are independently CH₂ or both hydrogens on the methylene linkage can be substituted to form a spiro system with or without the presence of heteroatoms selected from O, S(═O)ₙ where n is 0-2, or N—R₁ to form a five to eight membered cyclic system; W₂ is CH₂, N—R₁, O, S(═O)ₙ where n is 0-2; and t is 1-3; or

Z₃ is N; Y₁ is N; Y₂ is C; Z₁ is CR₂; and Z₂ is a carbon bonded to the remainder of the molecule; or

Z₃ is N; Y₁ is N; Y₂ is C; Z₁ is CR₂; Z₂ is a carbon bonded to the remainder of the molecule; W₁, W₂ and W₃ are independently CR₄R₄; and t is 1-3; or

Z₃ is N; Y₁ is N; Y₂ is C; Z₁ is CR₂; Z₂ is a carbon bonded to the remainder of the molecule; W₁ and W₃ are independently CR₄R₄; one of W₂ is N—R₁, O, or S(═O)ₙ where n is 0-2; and another W₂ is CR₄R₄; and t is 1-3; or

Z₃ is N; Y₁ is N; Y₂ is C; Z₁ is CR₂; Z₂ is a carbon bonded to the remainder of the molecule; W₁ and W₂ are independently CR₄R₄; W₃ is N—R₁, O, or S(═O)ₙ where n is 0-2; and t is 2; or

Z₃ is N; Y₁ is N; Y₂ is C; Z₁ is CR₂; Z₂ is a carbon bonded to the remainder of the molecule; W₁ and W₃ are independently CR₄R₄, W₂ is N—R₁, O, or S(═O)ₙ where n is 0-2; and t is 1; or

Z₂ is N; Y₁ is N; Y₂ is C; Z₃ is CR₂; Z₁ is a carbon bonded to the remainder of the molecule; W₁ and W₂ is CH₂ or both hydrogens on the methylene linkage can be substituted to form a spiro system with or without the presence of hetero atoms selected from O, S(═O)ₙ where n is 0-2, or N—R₁ to form a five- to eight-membered cyclic system; W₃ is N—R₁, O, or S(═O)ₙ where n is 0-2; and t is 3; or

Z₂ is N; Y₁ is N; Y₂ is C; Z₃ is CR₂; Z₁ is a carbon bonded to the remainder of the molecule; W₁ and W₃ are independently CH₂ or both hydrogens on the methylene linkage can be substituted to form a spiro system with or without the presence of heteroatoms selected from O, S(═O)ₙ where n is 0-2, or N—R₁ to form a five- to eight-membered cyclic system; one W₂ is N—R₁, O, or S(═O)ₙ where n is 0-2; another W₂ is CR₄R₄; and t is 2; or

Z₂ is N; Y₁ is N; Y₂ is C; Z₃ is CR₂; Z₁ is a carbon bonded to the remainder of the molecule; W₁ and W₃ are independently CH₂ or both hydrogens on the methylene linkage can be substituted to form a spiro system with or without the presence of heteroatoms selected from O, S(═O)ₙ where n is 0-2, or N—R₁ to form a five- to eight-membered cyclic system; W₂ is N—R₁, O, or S(═O)ₙ where n is 0-2; and t is 1; or

Z₂ is N; Y₁ is N; Y₂ is C; Z₁ is CR₂; Z₃ is a carbon bonded to the remainder of the molecule; W₁ and W₃ are independently CR₄R₄; one W₂ is N—R₁, O, or S(═O)ₙ where n is 0-2; another W₂ is CR₄R₄; and t is 3; or

Z₂ is N; Y₁ is N; Y₂ is C; Z₁ is CR₂; Z₃ is a carbon bonded to the remainder of the molecule; W₁ and W₃ are independently CR₄R₄; one W₂ is N—R₁, O or S(═O)ₙ where n is 0-2; another W₂ is CR₄R₄; and t is 2; or

Z₂ is N; Y₁ is N; Y₂ is C; Z₁ is CR₂; Z₃ is a carbon bonded to the remainder of the molecule; W₁ and W₃ are independently CR₄R₄; W₂ is N—R₁, O or S(═O)ₙ where n is 0-2; and t is 1; or

₁ and Z₂ are N; Y₁ is N; Y₂ is C; Z₃ is a carbon bonded to the remainder of the molecule; W₁ and W₃ are independently CR₄R₄; one of W₂ is N—R₁, O or S(═O)ₙ where n is 0-2; another W₂ is CR₄R₄; and t is 3; or

Z₁ and Z₂ are N; Y₁ is N; Y₂ is C; Z₃ is a carbon bonded to the remainder of the molecule; W₁ and W₃ are independently CR₄R₄; one of W₂ is N—R₁, O or S(═O)ₙ where n is 0-2; another W₂ is CR₄R₄; and t is 2; or

Z₁ and Z₂ are N; Y₁ is N; Y₂ is C; Z₃ is a carbon bonded to the remainder of the molecule; W₁ and W₃ are independently CR₄R₄; W₂ is N—R₁, O, or S(═O)ₙ where n is 0-2; and t is 1; or

Z₁ and Z₂ are independently CR₂; Y₁ is C; Y₂ is N; Z₃ is a carbon bonded to the remainder of the molecule; W₁ and W₃ are independently CR₄R₄; one W₂ is N—R₁, O or S(═O)ₙ where n is 0-2; another W₂ is CR₄R₄; and t is 3; or

Z₁ and Z₂ are independently CR₂; Y₁ is C; Y₂ is N; Z₃ is a carbon bonded to the remainder of the molecule; W₁ and W₃ are independently CR₄R₄; one W₂ is N—R₁, O, or S(═O)ₙ where n is 0-2; another W₂ is CR₄R₄; and t is 2; or

Z₁ and Z₂ are independently CR₂; Y₁ is C; Y₂ is N; Z₃ is a carbon bonded to the remainder of the molecule; W₁ and W₃ are independently CR₄R₄; W₂ is N—R₁, O, or S(═O)ₙ where n is 0-2; and t is 1; or

Z₁ and Z₃ are independently CR₂; Y₁ is C; Y₂ is N; Z₂ is a carbon bonded to the remainder of the molecule; W₁ and W₃ are independently CR₄R₄; one W₂ is N—R₁, O or S(═O)ₙ where n is 0-2; another W₂ is CR₄R₄; and t is 3; or

Z₁ and Z₃ are independently CR₂; Y₁ is C; Y₂ is N; Z₂ is a carbon bonded to the remainder of the molecule; W₁ and W₃ are independently CR₄R₄; one W₂ is N—R₁, O or S(═O)ₙ where n is 0-2; another W₂ is CR₄R₄; and t is 2; or

Z₁ and Z₃ are independently CR₂; Y₁ is C; Y₂ is N; Z₂ is a carbon bonded to the remainder of the molecule; W₁ and W₃ are independently CR₄R₄; W₂ is N—R₁, O or S(═O)ₙ where n is 0-2; and t is 1; or

Z₃ and Z₂ are independently CR₂; Y₁ is C; Y₂ is N; Z₁ is a carbon bonded to the remainder of the molecule; W₁ and W₂ are independently CR₄R₄; one W₂ is N—R₁, O, or S(═O)ₙ where n is 0-2; another W₂ is CR₄R₄; and t is 3; or

Z₃ and Z₂ are independently CR₂; Y₁ is C; Y₂ is N; Z₁ is a carbon bonded to the remainder of the molecule; W₁ and W₃ are independently CR₄R₄; one W₂ is N—R₁, O, or S(═O)ₙ where n is 0-2; another W₂ is CR₄R₄; and t is 2; or

Z₃ and Z₂ are independently CR₂; Y₁ is C; Y₂ is N; Z₁ is a carbon bonded to the remainder of the molecule; W₁ and W₃ are independently CR₄R₄; W₂ is N—R₁, O or S(═O)ₙ where n is 0-2; and t is 1; or

Z₃ is N; Y₁ is N; Y₂ is C; one of Z₁ and Z₂ is CR₂; the other Z is C; W₁ is CR₄R₄; W₂ is CR₄R₄; W₃ is CH₂, N—R₁ or O; and t is 1; or

Z₃ is N; Y₁ is N; Y₂ is C; one of Z₁ and Z₂ is CR₂; the other Z is C; W₁ is CR₄R₄; W₂ is C(═O); W₃ is N—R₁; and t is 1; or

Z₃ is N; Y₁ is N; Y₂ is C; one of Z₁ and Z₂ is CR₂; the other Z is C; W₁ is N—R₁; W₂ is C(═O); W₃ is CR₄R₄; and t is 1; or

Z₃ is N; Y₁ is N; Y₂ is C; one of Z₁ and Z₂ is CR₂; the other Z is C; W₁ is C(═O); W₂ is N—R₁; W₃ is CH₂; t is 1; and R₁, R₂, R₄, R₆, and R₇ are as defined above.

In Some Embodiments of the Formula 1-C:

Z₁, Z₂, Z₃, and Z₄ are independently CR₂; any one of Z₁, Z₂, Z₃, and Z₄ is a carbon bonded to the remainder of the molecule; Y₁ and Y₂ are C; W₁, W₂, and W₃ are independently CR₄R₄, S, SO, SO₂, O, or N—R₁; and t is 1-3; or

Z₁, Z₂, Z₃, and Z₄ are independently CR₂; any one of Z₁, Z₂, Z₃, and Z₄ is a carbon bonded to the remainder of the molecule; Y₁ and Y₂ is C or N; W₁, W₂, and W₃ are independently CR₄R₄, S, SO, SO₂, O, or N—R₁; and t is 1-3; or

Z₁, Z₂, Z₃, and Z₄ are independently CR₂; Y₁ and Y₂ are N; W₁, W₂, and W₃ are independently CR₄R₄, S, SO, SO₂, O, or N—R₁; and t is 1-3; or

Z₁ is N; Z₂, Z₃, and Z₄ are independently CR₂; Y₁ and Y₂ are C; W₁, W₂ and W₃ are independently CR₄R₄, S, SO, SO₂, O, or N—R₁; and t is 1-3; or

Z₁ is N; Z₂, Z₃, and Z₄ are independently CR₂; any one of Z₁, Z₂, Z₃, and Z₄ is a carbon bonded to the remainder of the molecule; Y₁ is C; Y₂ is N; W₁, W₂, and W₃ are independently CR₄R₄, S, SO, SO₂, O, or N—R₁; and t is 1-3; or

Z₂ is N; Z₁, Z₃, and Z₄ are independently CR₂; any one of Z₁, Z₂, Z₃, and Z₄ is a carbon bonded to the remainder of the molecule; Y₁ and Y₂ are C; W₁, W₂, and W₃ are independently CR₄R₄, S, SO, SO₂, O, or N—R₁; and t is 1-3; or

Z₂ is N; Z₁, Z₃, and Z₄ are independently CR₂; any one of Z₁, Z₂, Z₃,and Z₄ is a carbon bonded to the remainder of the molecule; Y₁ is C; Y₂ is N; W₁, W₂ and W₃ are independently CR₄R₄, S, SO, SO₂, O, or N—R₁; and t is 1-3; or

Z₃ is N; Z₁, Z₂, and Z₄ are independently CR₂; any one of Z₁, Z₂, Z₃ and Z₄ is a carbon bonded to the remainder of the molecule; Y₁ and Y₂ are C; W₁, W₂, and W₃ are independently CR₄R₄, S, SO, SO₂, O, or N—R₁; and t is 1-3; or

Z₃ is N; Z₁, Z₂, and Z₄ are independently CR₂; any one of Z₁, Z₂, Z₃, and Z₄ is a carbon bonded to the remainder of the molecule; Y₁ is C and Y₂ is N; W₁, W₂, and W₃ are independently CR₄R₄, S, SO, SO₂, O, or N—R₁; and t is 1-3; or

Z₄ is N; Z₁, Z₂, and Z₃ are independently CR₂; any one of Z₁, Z₂, Z₃, and Z₄ is a carbon bonded to the remainder of the molecule; Y₁ and Y₂ are C; W₁, W₂, and W₃ are independently CR₄R₄, S, SO, SO₂, O, or N—R₁; and t is 1-3; or

Z₄ is N; Z₁, Z₂, and Z₃ are independently CR₂; any one of Z₁, Z₂, Z₃, and Z₄ is a carbon bonded to the remainder of the molecule; Y₁ is N; Y₂ is C; W₁, W₂ and W₃ are independently CR₄R₄, S, SO, SO₂, O, or N—R₁; and t is 1-3; or

Z₁ is N; Z₂, Z₃, and Z₄ are independently CR₂; one of Z₁, Z₂, Z₃, and Z₄ is a carbon bonded to the remainder of the molecule; Y₁ and Y₂ are C; W₁, W₂, and W₃ are independently CR₄R₄, S, SO, SO₂, O, or N—R₁; and t is 1-3; or

Z₁ and Z₂ are N; Z₃ and Z₄ are independently CR₂; any one of Z₁, Z₂, Z₃, and Z₄ is a carbon bonded to the remainder of the molecule; Y₁ is C; Y₂ is N; W₁, W₂ and W₃ are independently CR₄R₄, S, SO, SO₂, O, or N—R₁; and t is 1-3; or

Z₁ and Z₃ are N; Z₂ and Z₄ are independently CR₂; any one of Z₁, Z₂, Z₃ and Z₄ is a carbon bonded to the remainder of the molecule; Y₁ is C; Y₂ is N; W₁, W₂, and W₃ are independently CR₄R₄, S, SO, SO₂, O, or N—R₁; and t is 1-3; or

Z₁ and Z₄ are N; Z₂ and Z₃ are independently CR₂; any one of Z₁, Z₂, Z₃ and Z₄ is a carbon bonded to the remainder of the molecule; Y₁ is N; Y₂ is C; W₁, W₂ and W₃ are independently CR₄R₄, S, SO, SO₂, O, or N—R₁; and t is 1-3; or

Z₁, Z₂, and Z₃ are N; Z₄ is a carbon bonded to the remainder of the molecule; Y₁ is C; Y₂ is N; W₁, W₂, and W₃ are independently CR₄R₄, S, SO, SO₂, O, or N—R₁; and t is 1-3; or

Z₁, Z₃, and Z₄ are N; Z₂ is a carbon bonded to the remainder of the molecule; Y₁ and Y₂ are C; W₁, W₂, and W₃ are independently CR₄R₄, S, SO, SO₂, O, or N—R₁; and t is 1-3; or

Z₁, Z₂, and Z₄ are N; Z₃ is a carbon bonded to the remainder of the molecule; Y₁ and Y₂ are C; W₁, W₂, and W₃ are independently CR₄R₄, S, SO, SO₂, O, or N—R₁; and t is 1-3; or

Z₂, Z₃, and Z₄ are N; Z₁ is a carbon bonded to the remainder of the molecule; Y₁ and Y₂ are C; W₁, W₂, and W₃ are independently CR₄R₄, S, SO, SO₂, O, or N—R₁; t is 1-3; and R₁, R₂, R₄, R₆, and R₇ are as defined above.

The expression ‘fused bicyclic heteroaryl group’ is used in the present application to mean:

A group comprising two fused rings in which one ring has aromatic character [i.e. Huckel's rule (4n+2)] and the other ring is non-aromatic.

The fused bicyclic heteroaryl group may contain one to six heteroatoms selected from O, S, N and N—R₁ and is bonded to the remainder of the molecule through a carbon atom in the aromatic ring.

The aromatic ring of the fused bicyclic heteroaryl group can contain five or six ring atoms (including bridgehead atoms) selected from CR₂, N, O, S, and N—R₁. The aromatic ring of the fused bicyclic heteroaryl group contains 0 to 3 heteroatoms selected from O, S, N, and N—R₁.

The non-aromatic ring of the fused bicyclic heteroaryl group can contain five to eight ring atoms (including bridgehead atoms) selected from CR₄R₄, N, N—R₁, O, and S(═O)ₙ where n is 0-2 and may contain 0-4 heteroatoms selected from N, N—R₁, O or S(═O)ₙ where n is 0-2.

Some examples of fused bicyclic heteroaryl, optionally substituted ring systems, are shown in Table 1:

TABLE 1

| | | |
|---|---|---|
| a) | 2,3-dihydroimidazo[2,1- b]thiazole | 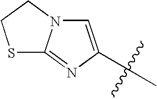 |
| b) | 4,5,6,7-tetrahydrothieno[3,2- c]pyridine | 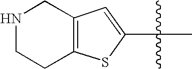 |
| c) | 5,6,7,8-tetrahydroimidazo[1,2- a]pyrazine | 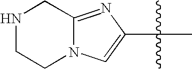 |
| d) | 5,6-dihydro-8H-imidazo[2,1- c][1,4]thiazine | 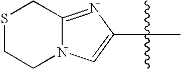 |
| e) | 6,7-dihydro-5H-pyrrolo[1,2- a]imidazole | 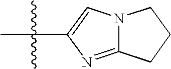 |
| f) | 5,6-dihydro-8H-imidazo[2,1- c][1,4]oxazine | 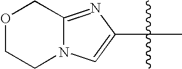 |
| g) | 5,6-dihydro-4H-pyrrolo[1,2- b]pyrazole | 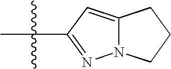 |
| h) | 4,5,6,7-tetrahydropyrazolo[1,5- a]pyridine | 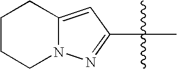 |
| i) | 7-methyl-6-oxo-5,6,7,8- tetrahydro-imidazo[1,2-a]pyrazin | 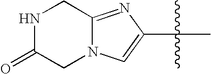 |
| j) | 6,7-dihydro-4H-pyrazolo[5,1- c][1,4]thiazine | 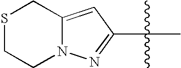 |
| k) | 4H-5-thia-1,6a-diazapentalene | 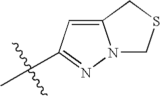 |
| l) | 7H-Imidazo[1,2-c]thiazole | 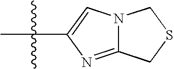 |
| m) | 6,7-dihydro-4H-thieno[3,2- c]pyran | 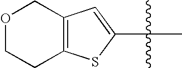 |
| n) | 6,7-dihydro-4H-thieno[3,2- c]thiopyran | 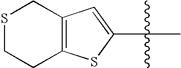 |
| o) | 6,7,8,9-tetrahydro-5H- imidazo[1,2-a]azepine | 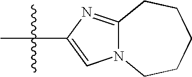 |
| p) | 5,5-dioxo-4,5,6,7-tetrahydro- 5λ⁶-pyrazolo[5,1-c][1,4]thiazine | 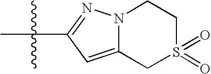 |
| q) | 4,5,6,7-tetrahydropyrazolo[1,5- a]pyrazine | 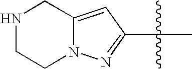 |
| r) | 5,6-dihydro-4H- cyclopenta[b]furan | 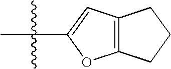 |
| s) | 4,5-dihydro-6-thia-1,7a- diazaindene | 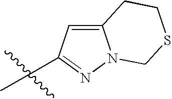 |
| t) | 2,3-dihydropyrazolo[5,1- b]thiazole | 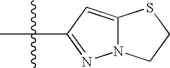 |
| u) | 2,3-dihydropyrazolo[5,1- b]oxazole | 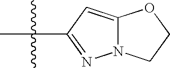 |
| v) | 6,7-dihydro-4H-pyrazolo[5,1- c][1,4]oxazine | 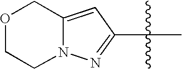 |

In some embodiments, the substituent 4,5,6,7-tetrahydrothieno[3,2-c]pyridine can be optionally substituted with a (C₆₋₂₀aryl)alkyl such as, benzyl; an alkoxyarylalkyl such as, 4-methoxybenzyl; a C₁₋₆alkyl such as, methyl; a heteroarylalkyl such as, pyridin-3-ylmethyl; an arylalkylC(═O) such as, phenylacetyl; a heteroarylC(═O) such as, pyridin-3-ylcarbonyl; or an alkylC(═O) such as, for example, acetyl.

In other embodiments, the substituent 5,6,7,8-tetrahydroimidazo[1,2-a]pyrazine can be optionally substituted with C₁₋₆alkyl such as, for example, methyl.

In other embodiments, the substituent 6,7,8,9-tetrahydro-5H-imidazo[1,2-a]azepine can be optionally substituted C₁₋₆alkyl such as, for example, methyl.

In other embodiments, the substituent 6,7-dihydro-4H-thieno[3,2-c]pyridine can be optionally substituted with C₁₋₆alkoxycarbonyl.

In other embodiments, the substituent 5,6,7,8-tetrahydroimidazo[1,2-a]pyrazine can be optionally substituted arylC1-6alkyl such as, benzyl.

In other embodiments, the substituent 4,5,6,7-tetrahydropyrazolo[1,5-a]pyrazine optionally substituted with alkoxyalkylC(═O) such as, for example, 2-methoxyacetyl; or by alkyloxyalkylC(═O) such as, for example, methoxyacetyl.

The expression “fused tricyclic heteroaryl group” is used in the present application to mean:

a group comprising three fused rings in which at least one ring has aromatic character (i.e. meets Huckel's rule (4n+2)). The fused tricyclic heteroaryl group contains 1-6 heteroatoms selected from the group consisting of O, S, N, and N—R₁. The fused tricyclic heteroaryl is bonded through a carbon in one of the at least one aromatic rings to the remainder of the molecule. The fused tricyclic heteroaryl group may contain 1-3 aromatic rings and 0-2 non-aromatic rings. Each aromatic ring(s) in the fused tricyclic heteroaryl group may contain 5 to 7 ring atoms (including the bridgehead atoms) selected from C, CR₂, O, S, N, and N—R₁. Each of the aromatic ring(s) of the fused tricyclic heteroaryl group may contain 0-3 heteroatoms selected from O, S, N, and N—R₁. The non-aromatic ring(s), if any, of the fused tricyclic heteroaryl group may contain 5-8 ring atoms (including bridgehead atoms) and contain 0-4 heteroatoms selected from N, N—R₁, O, or S(═O)ₙ, where n is 0-2. In each non-aromatic ring of the fused tricyclic heteroaryl group, one or two of the non-bridgehead carbon atoms may each be optionally substituted with one or two R₄, and each R₄ may be independently the same or different.

Examples of fused tricyclic heteroaryl, optionally substituted ring systems, are shown in Table 2:

TABLE 2

| | | |
|---|---|---|
| a) | 6,7-dihydro-5H- cyclopenta[d]imidazo[2,1- b][1,3]thiazole | 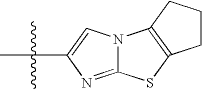 |
| b) | 5,6,7,8-tetrahydroimidazo[2,1- b][1,3]-benzothiazole | 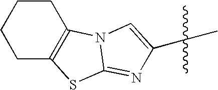 |
| c) | 5,8-dihydro-6H-imidazo[2,1- b]pyrano[4,3-d][1,3]thiazole | 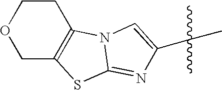 |
| d) | 4,5,6,7-tetrahydro-1,3a,3b,8- tetraaza-cyclopenta[a]indene | 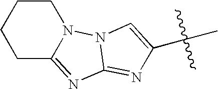 |
| e) | 5H-Imidazo[2,1-a]isoindole | 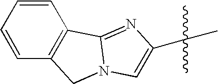 |
| f) | 5,6,7,8-tetrahydropyrazolo[5,1- b][1,3]benzoxazole | 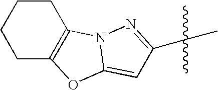 |
| g) | 7,8-dihydro-5H-pyrano[4,3- d]pyrazolo[5,1-b][1,3]oxazol | 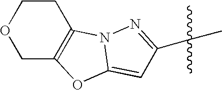 |
| h) | 5,6,7,8-tetrahydropyrazolo [5′,1′:2,3][1,3]oxazolo[5,4- c]pyridin | 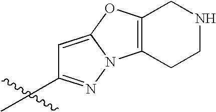 |
| i) | 7,8-dihydro-6H- cyclopenta[e]imidazo[1,2- a]pyrimidinyl | 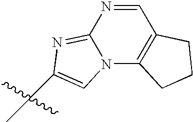 |
| j) | imidazo[1,2-a]quinoline | 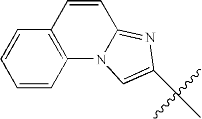 |
| k) | imidazo[1,2-a]quinoxaline | 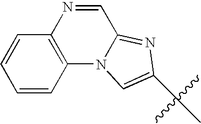 |
| l) | imidazo[2,1-b][1,3]benzothiazole | 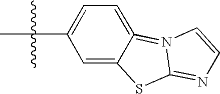 |
| m) | 2,3-dihydro[1,3]thiazolo[3,2- a]benzimidazole | 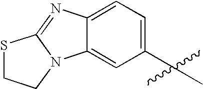 |
| n) | [1,3]thiazolo[3,2-a]benzimidazole | 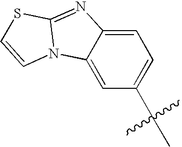 |
| o) | 3,4-dihydro-2H-[1,3]thiazino[3,2- a]benzimidazole | 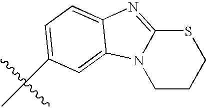 |
| p) | benzo[d]thiazolo[3,2-a]imidazole | 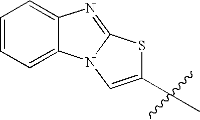 |
| q) | 7,8-dihydro-6H- cyclopenta[e][1,2,4]-triazolo[1,5- a]pyrimidine | 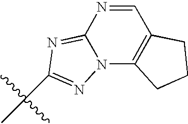 |
| r) | 9H-imidazo[1,2-a]benzimidazole | 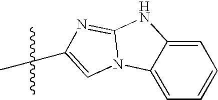 |
| s) | 7,8-dihydro-6H- cyclopenta[e]imidazo[1,2- a]pyrimidine | 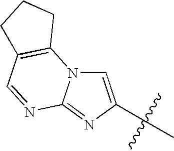 |
| t) | 7,8-dihydro-6H-3,4,8b-triaza-as- indacene | 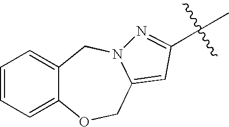 |
| u) | 7,8-dihydro-6H- cyclopenta[3,4]pyrazolo[5,1- b][1,3]thiazole | 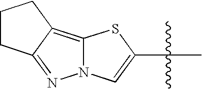 |
| v) | 8′,9′-dihydro-6′H-spiro[1,3- dioxolane-2,7′-[1,2,4]triazolo[1,5- a]-quinazoline | 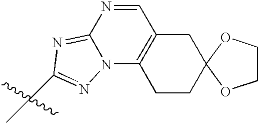 |
| w) | 6,7,8,9- tetrahydro[1,2,4]triazolo[1,5- a]quinazoline | 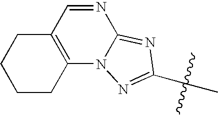 |
| x) | 7,8-dihydro-6H- cyclopenta[e]imidazo[1,2- a]pyrimidine | 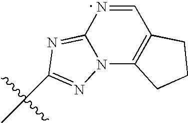 |
| y) | 4-dihydro-2H-[1,3]thiazino[3,2-a]- benzimidazole | 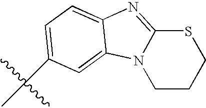 |
| z) | 7,8-dihydro-5H-pyrano[4,3- d]pyrazolo[5,1-b][1,3]-oxazole | 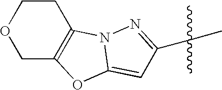 |

In an embodiment, the substituent imidazo[2,1-b][1,3]benzothiazole can be optionally substituted with C₁₋₆alkyl, C₁₋₆alkoxy or halo (such as, for example, chlorine or fluorine).

In some embodiments, the substituent 7,8-dihydro-6H-cyclopenta[e][1,2,4]-triazolo[1,5-a]pyrimidine can be optionally substituted with arylC1-6alkyl such as, for example, benzyl.

In some embodiments, the substituent 5,6,7,8-tetrahydro-[1,2,4]triazolo[1,5-a]pyridine dibenzo[b,f][1,4]-oxazepin-11(10H)-andone can be optionally substituted with C₁₋₆alkoxy.

In some embodiments, the substituent 7,8-dihydro-6H-3,4,8b-triaza-as-indacene can be optionally substituted with C₁₋₆alkoxy.

In some embodiments, the substituent 9H-imidazo[1,2-a]benzimidazole can be optionally substituted with C₁₋₆alkyl or 4H-thieno[2′,3′:4,5]thiopyrano[2,3-b]pyridine.

In some embodiments, the substituent 7,8-dihydro-6H-cyclopenta[e][1,2,4]-triazolo[1,5-a]pyrimidine and be optionally substituted with C₁₋₆alkyl.

In some embodiments, the substituent 6,7,8, 9-tetrahydropyrido[3,4-e][1,2,4]triazolo[1,5-a]pyrimidine can be optionally substituted with C₁₋₆alkoxycarbonyl.

In some embodiments, the substituent 6,7,8,9-tetrahydro[1,2,4]triazolo[1,5-a]quinazoline can be optionally substituted with C₁₋₆alkyl.

In other embodiments, the substituent 7,8-dihydro-6H-cyclopenta[e]imidazo[1,2-a]pyrimidine can be optionally substituted with C₁₋₆alkoxy.

In other embodiments, the substituent 7,8-dihydro-6H-cyclopenta[e]imidazo[1,2-a]pyrimidinyl can be optionally substituted with, for example, arylalkyloxyalkyloxy.

In other embodiments, the substituent 5,6,7,8-tetrahydropyrazolo[5′,1′:2,3][1,3]oxazolo [5,4-c]pyridine can be optionally substituted with, for example, C₁₋₆alkoxycarbonyl.

Further examples of tricyclic heteroaryl group A and B are as follows:

Ring Size and Arrangements: (5-5-5)

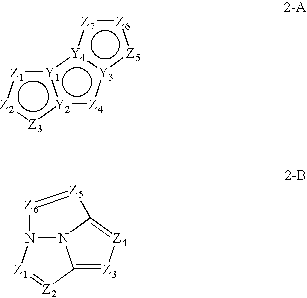

In both formula 2-A and 2-B: Z₁, Z₂, Z₃, Z₄, Z₅, Z₆ and Z₇ are independently CR₂, N, O, S, or N—R₁; any one of Z₁, Z₂, Z₃, Z₄, Z₅, Z₆, and Z₇ is a carbon atom to which the remainder of the molecule is attached; with the proviso that in formula 2-B, Z₁, Z₂, Z₃, Z₄, Z₅, Z₆ and Z₇ are selected from CR₂, N, and S;

Y₁, Y₂, Y₃ and Y₄ may independently be C or N;

R₁ is H, —C₁₋₆alkyl, -aryl, -heteroaryl or mono or bicyclic saturated heterocyclyl having 1-3 heteroatoms independently selected from O, N or S, —C₃₋₇cycloalkyl, —C₂₋₈alkenyl, —C₂₋₆alkynyl with the proviso that both the double bond and the triple bond should not be present at the carbon atom which is directly linked to N; —C1-C6 per fluoro alkyl, —S(═O)ₚ alkyl or aryl where p is 2, —C═Oheteroaryl, —C═Oaryl, —C═O (C1-C6) alkyl, —C(═O)C₃₋₆cycloalkyl, —C(═O)mono or bicyclic saturated heterocyclyl having 1-3 heteroatoms independently selected from O, N or S, C1-C6 alkyl aryl, C1-C6 alkyl heteroaryl, aryl-C1-C6 alkyl, heteroaryl-C1-C6 alkyl, C1-C6 alkyl mono or bicyclic saturated heterocyclyl having 1-3 heteroatoms independently selected from O, N or S, arylalkenyl of 8 to 16 carbon atoms, —CONR₆R₇, —SO₂NR₆R₇, arylalkyloxyalkyl, -alkyl-O-alkyl-aryl, -alkyl-O-alkyl-heteroaryl, aryloxyalkyl, heteroaryloxyalkyl, aryloxyaryl, aryloxyheteroaryl, C1-C6alkyl aryloxyaryl, C1-C6 alkyl aryloxyheteroaryl , alkyl aryloxy alkylamines, C₁₋₆alkoxycarbonyl, aryloxy carbonyl, heteroaryloxy carbonyl;

R₂ is absent, hydrogen, halogen, cyano, N—R₆R₇, hydroxy; COOR₆, C₁₋₆alkylamino (C₁₋₆alkoxy), alkylenedioxy, C₁₋₆perfluoroalkyl, CONR₆R₇, guanidino or cyclic guanidino, SO₂NR₆R₇, C₁₋₆alkyl, C₂₋₈alkenyl having 1 to 2 double bonds, C₂₋₆alkynyl having 1 to 2 triple bonds; C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxy, alkylaryloxy alkylamines, aryloxy, heteroaryloxy, alkenyloxy, alkynyloxy, aryloxyalkyl amine, C₁₋₆perfluoroalkyl, S(═O)_{q}—C₁₋₆alkyl, S(═O)_{q}— where q is 0-2, alkylaryl, arylalkyl, C₁₋₆alkylheteroaryl, heteroaryl-C₁₋₆alkyl, C₁₋₆alkyl mono or bicyclic saturated heterocyclyl having 1-3 heteroatoms independently selected from O, N or S, arylalkenyl of 8 to 16 carbon atoms, arylalkyloxyalkyl, aryloxyalkyl, heteroaryloxyalkyl, aryloxyaryl, aryloxyheteroaryl, heteroaryloxyaryl, alkyl aryloxyaryl, alkylaryloxyheteroaryl, aryloxyalkyl, heteroaryloxyalkyl, alkylaryloxyalkylamines, C₃₋₇cycloalkyl, saturated or partially saturated heterocyclyl, said group being optionally substituted with nitro, C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl, C₁₋₆alkoxy, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, C₁₋₆alkyl-S—, —SO₂N—R₆R₇, C₆₋₁₄aryl-O—, heteroaryl-O—, —S((═O)ₛ-aryl where s is 0-2, -alkyl-O-alkyl-NR₆R₇, -alkyl-aryl-O-alkylN—R₆R₇, -alkyl-aryl-O-alkylN—R₅R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; provided that the atom of R₂ that bonds to N is not a carbon that is double or triple bonded to another carbon;

R₄ is H, optionally substituted C₁₋₆ alkyl, one of R₄ is OH, C₁₋₆alkoxy, —S—C₁₋₆ alkyl, COOR₆, —NR₆R₇, —CONR₆R₇; or R₄R₄ may together be (═O) or R₄R₄ together with the carbon to which they are attached may form a spiro system of five to eight members with or without the presence of heteroatoms selected N, O, S(═O)ₙ (where n=0 to 2), N—R₁;

R₆ and R₇ are independently H, or a group selected from C₁₋₆alkyl, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, alkylaryl, arylalkyl, heteroarylalkyl, C₁₋₆alkylheteroaryl, said group being optionally substituted with nitro, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl-, C₁₋₆alkoxy, -alkoxyalkyl, alkyl-O—C₂₋₄alkyl-O-cyano, -halogen, -hydroxy, —NR₆R₇, —COOH, —COO-alkyl, -trifluoromethyl, -trifluoromethoxy, arylalkyl, alkylaryl, R₆R₇N-alkyl-, alkoxyalkyl-, C₁₋₆alkyl-S—, —SO₂NHR₆, —CO₂H, CONR₆R₇, C₆₋₁₄aryl-O—, heteroaryl-O—, —S(═O)ₛ-aryl, wherein s is 0-2, -alkyl-O-alkyl-NR₆R₇, -alkyl-aryl-O-alkyl-N—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl, wherein S is 0-2, or R₆ and R₇ can together with the nitrogen to which they are attached form a 3 to 7 membered saturated ring system optionally having one or two heteroatoms selected from N—R₁, O, and S(═O)ᵣ, where r is 0-2.

Ring Size and Arrangement: (5-5-6)

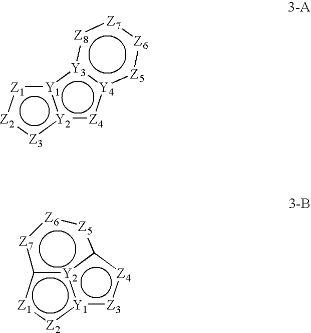

In both formula 3-A and 3-B: Z₁, Z₂, Z₃, Z₄, Z₅, Z₆, Z₇ and Z₈ are independently CR₂, N, O, S, or N—R₁; any one of Z₁, Z₂, Z₃, Z₄, Z₅, Z₆, Z₇, and Z₈ is a carbon atom to which the remainder of the molecule is attached; Y₁, Y₂, Y₃ and Y₄ are independently C or N; and

R₁, R₂, R₄, R₆ and R₇ are as defined above.

Ring Size and Arrangement: (5-6-5)

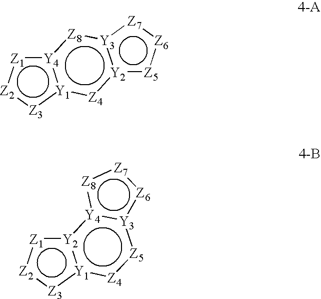

In both formula 4-A and 4-B: Z₁, Z₂, Z₃, Z₄, Z₅, Z₆, Z₇, and Z₈ are independently CR₂, N, O, S, or N—R₁; any one of Z₁, Z₂, Z₃, Z₄, Z₅, Z₆, Z₇, and Z₈ is a carbon atom to which the remainder of the molecule is attached; Y₁, Y₂, Y₃, and Y₄ are independently C or N; and

R₁, R₂, R₄, R₆ and R₇ are as defined above.

Ring Size and Arrangements: (5-6-6)

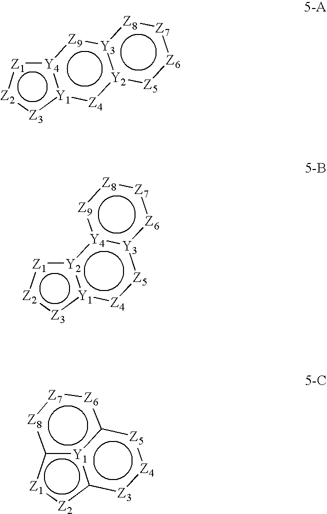

In formula 5-A, 5-B and 5-C: Z₁, Z₂, Z₃, Z₄, Z₅, Z₆, Z₇, and Z₈ are independently CR₂, N, O, S, or N—R₁; any one of Z₁, Z₂, Z₃, Z₄, Z₅, Z₆, Z₇, and Z₈ is a carbon atom to which the remainder of the molecule is attached; Y₁, Y₂, Y₃, and Y₄ are independently C or N; and

R₁, R₂, R₄, R₆ and R₇ are as defined above.

Ring Size and Arrangements: [5-5-(Non-Aromatic)]

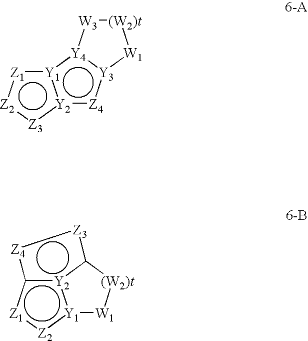

In both formula 6-A and 6-B: Z₁, Z₂, Z₃, and Z₄ are independently CR₂, N, O, S, or N—R₁; any one of Z₁, Z₂, Z₃, and Z₄ is a carbon atom to which the remainder of the molecule is attached; Y₁, Y₂, Y₃ and Y₄ are independently C or N; W₁, W₂, and W₃ are independently CR₄R₄, S(═O)r where r is 0-2, O, or N—R₁; with the proviso that they form no S—S, S—O, or O—O; t is 1-3; and

R₁, R₂, R₄, R₆ and R₇ are as defined above.

Ring Size and Arrangement: [5-6-(Non-Aromatic)]

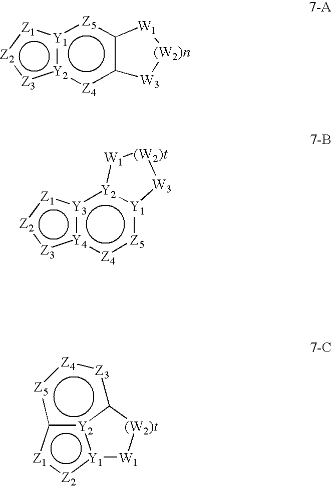

In formula 7-A, 7-B and 7-C: Z₁, Z₂, Z₃, Z₄ and Z₅ are independently CR₂, N, O, S or N—R₁; any one of Z₁, Z₂, Z₃, Z₄ and Z₅ is a carbon atom to which the remainder of the molecule is attached; Y₁ and Y₂ are independently C or N; W₁, W₂, and W₃ are independently CR₄R₄, S(═O)r where r is 0-2, O, or N—R₁; with the proviso that whenever W2 is S or O it is not adjacent to a W₁, W₂, and W₃ that is also S or O; t is 1-3; and

R₁, R₂, R₄, R₆ and R₇ are as defined above.

Ring Size and Arrangement: [5-(Non-Aromatic)-5]

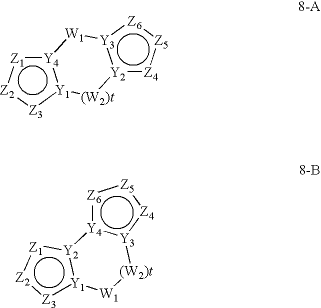

In formula 8-A and 8-B: Z₁, Z₂, Z₃, Z₄, Z₅ and Z₆ are independently CR₂N, O, S, and N—R₁; any one of Z₁, Z₂, Z₃, Z₄, Z₅ and Z₆ is a carbon atom to which the remainder of the molecule is attached; Y₁, Y₂, Y₃, and Y₄ are independently C or N; W₁ and W₂ are independently CR₄R₄, S(═O)r where r is 0-2, O, or N—R₁; with the proviso that whenever W₂ is S or O it is not adjacent to a W₁, W₂, and W₃ that is also S or O; t is 1-3; and

R₁, R₂, R₄, R₆ and R₇ are as defined above.

Ring Size and Arrangement: [5-(Non-Aromatic)-6]

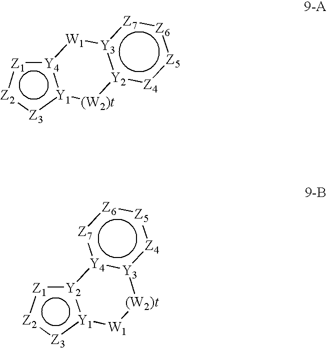

In formula 9-A and 9-B: Z₁, Z₂, Z₃, Z₄, Z₅, Z₆ and Z₇ are independently CR₂, N, O, S, or N—R₁; any one of Z₁, Z₂, Z₃, Z₄, Z₅, Z₆, and Z₇ is a carbon atom to which the remainder of the molecule is attached; Y₁, Y₂, Y₃, and Y₄ are independently C or N; W₁ and W₂ are independently CR₄R₄, S(═O)r where r is 0-2, O, or N—R₁; with the proviso that whenever W2 is S or O it is not adjacent to a W₁, W₂, and W₃ that is also S or O; t is 0-3; and

R₁, R₂, R₄, R₆ and R₇ are as defined above.

Ring Size and Arrangement [5-(Non-Aromatic)-(Non-Aromatic)]

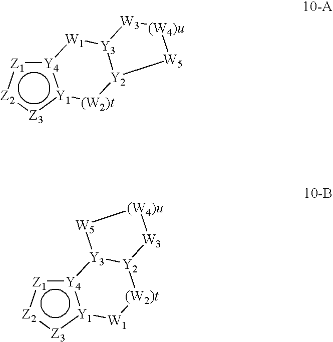

In formula 10-A and 10-B: Z₁, Z₂ and Z₃ are independently CR₂, N, O, S, or N—R₁; any one of Z₁, Z₂, and Z₃ is a carbon atom to which the remainder of the molecule is attached; Y₁ and Y₄ are independently C or N; Y₂ and Y₃ are independently CH or N; W₁, W₂, W₃, W₄ and W₅ are independently CR₄R₄, S(═O)r where r is 0-2, O, or N—R₁; with the proviso that whenever W2 is S or O it is not adjacent to a W₁, W₂ or W₃ that is also S or O with the proviso that whenever W2 is S or O it is not adjacent to a W₁, W₂, and W₃ that is also S or O; t is 0-2; u is 1-3; and

R₁, R₂, R₄, R₆ and R₇ are as defined above.

Ring Size and Arrangement (6-5-6)

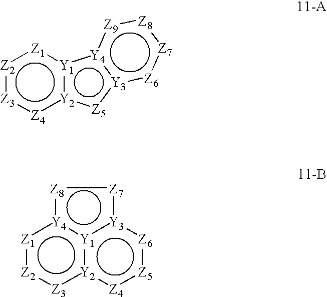

In formula 11-A and 11-B: Z₁, Z₂, Z₃, Z₄, Z₅, Z₆, Z₇, Z₈, and Z₉ are independently CR₂, N, O, S, or N—R₁; any one of Z₁, Z₂, Z₃, Z₄, Z₅, Z₆, Z₇, Z₈, and Z₉ is a carbon atom to which the remainder of the molecule is attached; and Y₁, Y₂, Y₃, and Y₄ are independently C or N; and

R₁, R₂, R₄, R₆ and R₇ are as defined above.

Ring Size and Arrangement (6-6-6)

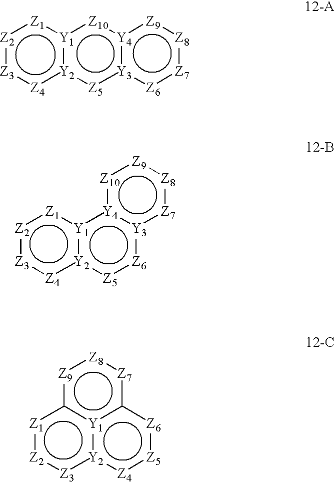

In formula 12-A, 12-B and 12-C: Z₁, Z₂, Z₃, Z₄, Z₅, Z₆, Z₇, Z₈, Z₉, and Z₁₀ are independently CR₂, N, O, S or N—R₁; any one of Z₁, Z₂, Z₃, Z₄, Z₅, Z₆, Z₇, Z₈, Z₉, and Z₁₀ is a carbon atom to which the remainder of the molecule is attached; and Y₁, Y₂, Y₃, and Y₄ are independently C or N; and

R₁, R₂, R₄, R₆ and R₇ are as defined above.

Ring Size and Arrangement [6-5-(Non-Aromatic)]

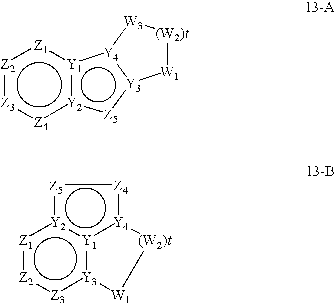

In formula 13-A and 13-B: Z₁, Z₂, Z₃, Z₄ and Z₅ are independently CR₂, N, O, S, or N—R₁; any one of Z₁, Z₂, Z₃, Z₄ and Z₅ is a carbon atom to which the remainder of the molecule is attached; Y₁, Y₂, Y₃ and Y₄ are independently C or N; W₁, W₂, and W₃ are independently CR₄R₄, O, S(═O)ᵣ where r is 0-2, or N—R₁; with the proviso that whenever W₂ is S or O it is not adjacent to a W₁, W₂ or W₃ that is also S or O, with the proviso that whenever W2 is S or O it is not adjacent to a W₁, W₂, and W₃ that is also S or O; t is 1-4; and

R₁, R₂, R₄, R₆ and R₇ are as defined above.

Ring Size and Arrangement [6-6-(Non-Aromatic)]

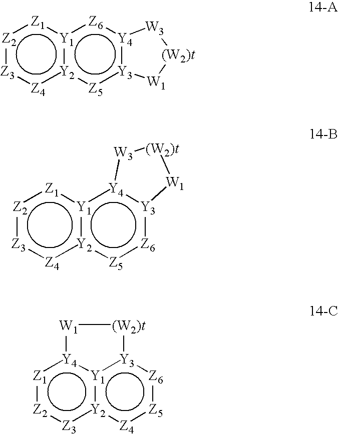

In formula 14-A, 14-B and 14-C: Z₁, Z₂, Z₃, Z₄, Z₅, and Z₆ are independently CR₂, N, O, S, or N—R₁; any one of Z₁, Z₂, Z₃, Z₄, Z₅, and Z₆ is a carbon atom to which the remainder of the molecule is attached; Y₁, Y₂, Y₃, and Y₄ are independently C or N; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and

R₁, R₂, R₄, R₆ and R₇ are as defined above.

Ring Size and Arrangement [6-(Non-Aromatic)-6]

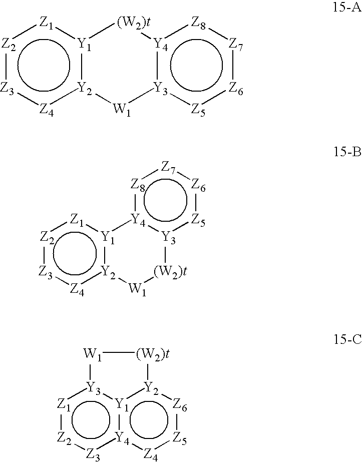

In formula 15-A, 15-B and 15-C: Z₁, Z₂, Z₃, Z₄, Z₅, Z₆, Z₇ and Z₈ are independently CR₂, N, O, S, or N—R₁; any one of Z₁, Z₂, Z₃, Z₄, Z₅, Z₆, Z₇ and Z₈ is a carbon atom to which the remainder of the molecule is attached; Y₁, Y₂, Y₃, and Y₄ are independently C or N; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-2; and

R₁, R₂, R₄, R₆ and R₇ are as defined above.

Ring Size and Arrangement [6-(Non-Aromatic)-(Non-Aromatic)]

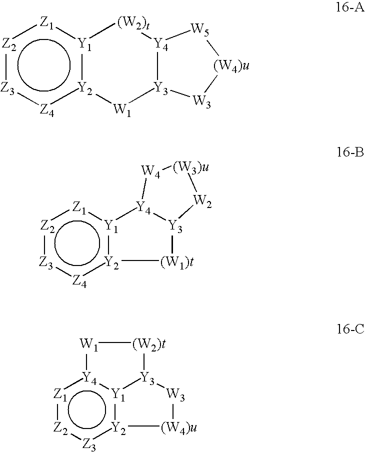

In formula 16-A, 16-B and 16-C: Z₁, Z₂, Z₃, and Z₄ are independently CR₂, N, O, S, or N—R₁; any one of Z₁, Z₂, Z₃, and Z₄ is a carbon atom to which the remainder of the molecule is attached; Y₁, Y₂, Y₃, and Y₄ are independently C or N; W₁, W₂, W₃, W₄, and W₅ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; u is 1-3; and

R₁, R₂, R₄, R₆ and R₇ are as defined above.

In Some Embodiments of Formula 2-A are:

Z₁ is O, S, or N—R₁; Z₂, Z₃, Z₄, Z₅, Z₆, and Z₇ are independently CR₂or N; Y₁, Y₂, Y₃, and Y₄, are C; and any one of Z₂, Z₃, Z₄, Z₅, Z₆, and Z₇ is a carbon to which the remainder of the molecule is attached; or

Z₁ is O, S, or N—R₁; Z₂, Z₃, Z₄, Z₅, Z₆, and Z₇ are independently CR₂; Y₁, Y₂, Y₃, and Y₄ are C; and any one of Z₂, Z₃, Z₄, Z₅, Z₆, and Z₇ is a carbon to which the remainder of the molecule is attached; or

Z₂ is O, S, or N—R₁; Z₁, Z₃, Z₄, Z₅, Z₆, and Z₇ are independently CR₂ or N; Y₁, Y₂, Y₃, and Y₄ are C; and any one of Z₁, Z₃, Z₄, Z₅, Z₆, and Z₇ is a carbon to which the remainder of the molecule is attached; or

Z₂ is O, S, or N—R₁; Z₁, Z₃, Z₄, Z₅, Z₆, and Z₇ are independently CR₂; Y₁, Y₂, Y₃, and Y₄ are C; and any one of Z₁, Z₃, Z₄, Z₅, Z₆, and Z₇ is a carbon to which the remainder of the molecule is attached; or

Z₃ is O, S, or N—R₁; Z₁, Z₂, Z₄, Z₅, Z₆, and Z₇ are independently CR₂ or N; Y₁, Y₂, Y₃, and Y₄ are C; and any one of Z₁, Z₂, Z₄, Z₅, Z₆, and Z₇ is a carbon to which the remainder of the molecule is attached; or

Z₃ is O, S, or N—R₁; Z₁, Z₃, Z₄, Z₅, Z₆, and Z₇ are independently CR₂; Y₁, Y₂, Y₃, and Y₄ are C; and any one of Z₁, Z₃, Z₄, Z₅, Z₆, and Z₇ is a carbon to which the remainder of the molecule is attached; or

Z₇ is O, S, or N—R₁; Z₁, Z₂, Z₃, Z₄, Z₅, and Z₆ are independently CR₂ or N; Y₁, Y₂, Y₃, Y₄ are C; and any one of Z₁, Z₂, Z₃, Z₄, Z₅ and Z₆ is a carbon to which the remainder of the molecule is attached; or

Z₇ is O, S, or N—R₁; Z₁, Z₂, Z₃, Z₄, Z₅, and Z₆ are independently CR₂; Y₁, Y₂, Y₃, and Y₄ are C; and any one of Z₁, Z₂, Z₃, Z₄, Z₅, and Z₆ is a carbon to which the remainder of the molecule is attached; or

Z₁, Z₄, and Z₆ are independently O, S, or N—R₁; Z₂, Z₃, Z₅, and Z₇ are independently CR₂ or N; Y₁, Y₂, Y₃, and Y₄ are C; and any one of Z₂, Z₃, Z₅, and Z₇ is a carbon to which the remainder of the molecule is attached; or

Z₁, Z₄, and Z₆ are independently O, S, or N—R₁; Z₂, Z₃, Z₅, and Z₇ are independently CR₂; Y₁, Y₂, Y₃, and Y₄ are C; and any one of Z₂, Z₃, Z₅, and Z₇ is a carbon to which the remainder of the molecule is attached; or

Z₃, Z₄, and Z₆ are independently O, S, or N—R₁; Z₁, Z₂, Z₅, and Z₇ are independently CR₂ or N; Y₁, Y₂, Y₃, and Y₄ are C; and any one of Z₁, Z₂, Z₅, and Z₇ is a carbon to which the remainder of the molecule is attached; or

Z₃, Z₄, and Z₆ are independently O, S, or N—R₁; Z₂, Z₃, Z₅, and Z₇ are independently CR₂; Y₁, Y₂, Y₃, and Y₄ are C; and any one of Z₁, Z₂, Z₅, and Z₇ is a carbon to which the remainder of the molecule is attached; or

Z₁ is O, S, or N—R₁; Z₂, Z₃, Z₄, Z₅, Z₆ and Z₇ are independently CR₂ or N; Y₂ is N; Y₁, Y₃, and Y₄ are C; and any one of Z₂, Z₃, Z₄, Z₅, Z₆, and Z₇ is a carbon to which the remainder of the molecule is attached; or

Z₁ is O, S, or N—R₁; Z₂, Z₃, Z₄, Z₅, Z₆ and Z₇ are independently CR₂; Y₂ is N; Y₁, Y₃, and Y₄ are C; and any one of Z₂, Z₃, Z₄, Z₅, Z₆, and Z₇ is a carbon to which the remainder of the molecule is attached; or

Z₂ and Z₄ are independently O, S, or N—R₁; Z₁, Z₃, Z₅, Z₆, and Z₇ are independently CR₂ N; Y₁ is N; Y₂, Y₃, and Y₄ are C; and any one of Z₁, Z₃, Z₅, Z₆, and Z₇ is a carbon to which the remainder of the molecule is attached; or

Z₂ and Z₄ are independently O, S, or N—R₁; Z₁, Z₃, Z₅, Z₆, and Z₇ are independently CR₂; Y₁ s N; Y₂, Y₃, and Y₄ are C; and any one of Z₁, Z₃, Z₅, Z₆, and Z₇ is a carbon to which the remainder of the molecule is attached; or

Z₃ and Z₅ are independently O, S, or N—R₁; Z₁, Z₂, Z₄, Z₆, and Z₇ are independently CR₂ or N; Y₁ is N; Y₂, Y₃, and Y₄ are C; and any one of Z₁, Z₃, Z₅, Z₆, and Z₇ is a carbon to which the remainder of the molecule is attached; or

Z₃ and Z₅ are independently O, S, or N—R₁; Z₁, Z₂, Z₄, Z₆, and Z₇ are independently CR₂; Y₁ is N; Y₂, Y₃, and Y₄ are C; and any one of Z₁, Z₃, Z₅, Z₆, and Z₇ is a carbon to which the remainder of the molecule is attached; or

Z₁ and Z₅ are independently O, S, or N—R₁; Z₂, Z₃, Z₄, Z₆, and Z₇ are independently N, or CR₂; Y₁ is N; Y₂, Y₃, and Y₄ are C; and any one of Z₂, Z₃, Z₄, Z₆, and Z₇ is a carbon to which the remainder of the molecule is attached; or

Z₁ and Z₅ are independently O, S, or N—R₁; Z₂, Z₃, Z₄, Z₆, and Z₇ are independently CR₂; Y₁ is N; Y₂, Y₃, and Y₄ are C; and any one of Z₂, Z₃, Z₄, Z₆, and Z₇ is a carbon to which the remainder of the molecule is attached; or

Z₃ and Z₇ are independently O, S, or N—R₁; Z₁, Z₂, Z₄, Z₅, and Z₆ are independently N or CR₂; Y₁ is N; Y₂, Y₃, and Y₄ are C; and any one of Z₁, Z₂, Z₄, Z₅, and Z₆ is a carbon to which the remainder of the molecule is attached; or

Z₃ and Z₇ are independently O, S, or N—R₁; Z₁, Z₂, Z₄, Z₅, and Z₆ are independently CR₂; Y₁ is N; Y₂, Y₃, and Y₄ are C; and any one of Z₁, Z₂, Z₄, Z₅, and Z₆ is a carbon to which the remainder of the molecule is attached; or

Z₃ and Z₇ are independently O, S, or N—R₁; Z₁, Z₂, Z₄, Z₅, and Z₆ are independently N or CR₂; Y₂ is N; Y₁, and Y₃, Y₄ are C; and any one of Z₁, Z₂, Z₄, Z₅, and Z₆ is a carbon to which the remainder of the molecule is attached; or

Z₃ and Z₇ are independently O, S, or N—R₁; Z₁, Z₂, Z₄, Z₅, and Z₆ are independently CR₂; Y₂ is N; Y₁, Y₃, and Y₄ are C; and any one of Z₁, Z₂, Z₄, Z₅, and Z₆ is a carbon to which the remainder of the molecule is attached; or

Z₃ and Z₅ are independently O, S, or N—R₁; Z₁, Z₂, Z₄, Z₆, and Z₇ are independently N or CR₂; Y₂ is N; Y₁, Y₃, and Y₄ are C; and any one of Z₁, Z₂, Z₄, Z₆, and Z₇ is a carbon to which the remainder of the molecule is attached; or

Z₃ and Z₅ are independently O, S, or N—R₁; Z₁, Z₂, Z₄, Z₆, and Z₇ are independently CR₂; Y₂ is N; Y₁, Y₃, and Y₄ are C; any one of Z₁, Z₂, Z₄, Z₆, and Z₇ is a carbon to which the remainder of the molecule is attached; and

R₁, R₂, R₄, R₆ and R₇ are as defined above.

In Another Embodiment of Formula 2-B is:

Z₁, Z₂, Z₃, Z₄, Z₅, Z₆ and Z₇ are independently CR₂; and

R₁, R₂, R₄, R₆ and R₇ are as defined above.

In Some Embodiments of Formula 3-A are:

Z₁ is CR₂; Z₂ is a carbon to which the remainder of the molecule is attached; Z₃ is N or CR₂; Z₄ is O, S, CR₂, or N—R₁; Z₅, Z₆, Z₇, and Z₈ are independently CR₂ or N; Y₁ is N; and Y₂, Y₃ and Y₄ are C; or

Z₂ is CR₂; Z₁ is a carbon to which the remainder of the molecule is attached; Z₃ is N or CR₂; Z₄ is O, S, CR₂ or N—R₁; Z₅Z₆, Z₇, and Z₈ are independently CR₂ or N; Y₁ is N; and Y₂, Y₃ and Y₄ are C; or

Z₁ is N; Z₂ is a carbon to which the remainder of the molecule is attached; Z₃ is N or CR₂; Z₄ is O, S, CR₂, or N—R₁; Z₆, Z₇, and Z₈ are independently CR₂ or N; Y₁ is N; and Y₂, Y₃ and Y₄ are C; or

Z₁, Z₂, Z₃ are independently CR₂ or N; Z₄ is O, S, CR₂ or N—R₁, Z₅, Z₆, Z₇, and Z₈ are independently CR₂ or N; one of Z₅, Z₆, Z₇, or Z₈ is a carbon to which the remainder of the molecule is attached; Y₁ is N; and Y₂, Y₃ and Y₄ is C; or

Z₁ is CR₂ or N; Z₂ is CR₂; Z₃ is O, S or N—R₁; Z₄ is N or CR₂; Z₅, Z₆, Z₇, and Z₈ are independently CR₂; Y₁ is N or C; and Y₂, Y₃ and Y₄ are C; or

Z₁, Z₂, Z₃, Z₄, Z₅, Z₆, Z₇, and Z₈ are independently N or CR₂; and Y₁, Y₂, Y₃, and Y₄ are C; or

Z₁, Z₂, Z₅, Z₆, Z₇, and Z₈ are independently N or CR₂; Z₃ and Z₄ are independently O, S, or N—R₁; and Y₁, Y₂, Y₃, and Y₄ are C; or

Z₁, Z₂, and Z₃ are independently CR₂ or N; Z₄ is O, S, CR₂, or N—R₁; Z₅, Z₆, Z₇, and Z₈ are independently CR₂ or N; Y₁ is N; and Y₂, Y₃ and Y₄ are C; or

Z₁ is N; Z₂ is CR₂; Z₃ is a carbon to which remainder of the molecule is attached; Z₄ is N; Z₅, Z₆, Z₇, and Z₈ are independently N or CR₂; Y₁, Y₂, Y₃, and Y₄ are independently N or C; and

R₁, R₂, R₄, R₆ and R₇ are as defined above.

In Some Embodiments of Formula 3-B are:

Z₁ and Z₄ are independently CR₂ or N; Z₂ and Z₃ are CR₂; Z₅, Z₆, and Z₇ are independently CR₂ or N; Y₁ is C; and Y₂ is N; or

Z₁ is O, S, or N—R₁; Z₂ is CR₂; Z₃ is CR₂ or N; Z₄ is O, S, N—R₁, or CR₂; Z₅, Z₆, Z₇, and Z₈ are independently N or CR₂; Y₁, Y₂, Y₃, and Y₄ are C; any one of Z₂, Z₃, Z₅, Z₆, Z₇, and Z₈ is a carbon atom to which the remainder of the molecule is attached; and

R₁, R₂, R₄, R₆ and R₇ are as defined above.

In Some Embodiments of Formula 4-A are:

Z₁ is O, S, or N—R₁; Z₂ is N or CR₂; Z₃ is CR₂; Z₅, Z₆, and Z₇ are independently N or CR₂; Z₄ and Z₈ are independently O, S, or N—R₁; Y₁, Y₂, Y₃, and Y₄ are C; and any one of Z₂, Z₅, Z₆, and Z₇ is a carbon atom to which the remainder of the molecule is attached; or

Z₃ is O, S, or N—R₁; Z₂ is N or CR₂; Z₁ is CR₂; Z₅, Z₆, and Z₇ are independently N or CR₂; Z₄ and Z₈ are independently O, S, or N—R₁; Y₁, Y₂, Y₃, and Y₄ are C; any one of Z₂, Z₅, Z₆, and Z₇ is a carbon atom to which the remainder of the molecule is attached; and

R₁, R₂, R₄, R₆ and R₇ are as defined above.

In Some Embodiments of Formula 4-B are:

Z₁ is O, S, or N—R₁; Z₂ is N or CR₂; Z₃ is CR₂; Z₄, Z₅, Z₆, Z₇, and Z₈ are independently N or CR₂; Y₁, Y₂, Y₃, and Y₄ are C; and any one of Z₂, Z₅, Z₆, and Z₇ is a carbon atom to which the remainder of the molecule is attached; or

Z₁ is N or CR₂; Z₂ is CR₂; Z₃ is O, S, N—R₁ or CR₂; Z₇ is CR₂ or N; Z₆ and Z₈ are independently N or CR₂; Z₄ and Z₅ are CR₂ or N; Y₁, Y₂, and Y₃ are C; Y₄ is N; and any one of Z₂, Z₄, Z₅, and Z₆ is a carbon atom to which the remainder of the molecule is attached; or

Z₁ is N or CR₂; Z₂ is CR₂; Z₃ is O, S, N—R₁ or CR₂; Z₆ is CR₂ or N; Z₇ and Z₈ are independently N or CR₂; Z₄ and Z₅ are independently CR₂ or N; Y₁, Y₂, and Y₃ are C; Y₄ is N; and any one of Z₂, Z₄, Z₅, and Z₆ is a carbon atom to which the remainder of the molecule is attached; or

Z₁ is O, S, or N—R₁; Z₂ is N or CR₂; Z₃ is CR₂; Z₆, Z₇, and Z₈ are N; Z₄ and Z₅ are independently CR₂ or N; Y₁, Y₂, and Y₄ are C; Y₃ is N; and one of Z₂ or Z₃ is a carbon atom to which the remainder of the molecule is attached; or

Z₁ is N or CR₂; Z₂ is CR₂; Z₃ is O, S, N—R₁ or CR₂; Z₆, Z₇, and Z₈ are N; Z₄ and Z₅ are independently CR₂ or N; Y₁, Y₂, and Y₄ are C; Y₃ is N; and one of Z₁ or Z₂ is a carbon atom to which the remainder of the molecule is attached; or

Z₁ is N or CR₂; Z₂ is CR₂; Z₃ is O, S, N—R₁ or CR₂; Z₆, Z₇, and Z₈ are N; Z₄ and Z₅ are independently CR₂ or N; Y₁, Y₂, and Y₄ are C; Y₃ is N; and one of Z₁ or Z₂ is a carbon atom to which the remainder of the molecule is attached; or

Z₁ is N; Z₂, Z₃, Z₄, and Z₅ are independently CR₂; Z₆, Z₇, and Z₈ are independently O, S, N, N—R₁ or CR₂; Y₂, Y₃, and Y₄ are C; Y₁ is N; and any one of Z₂, Z₃, Z₆, Z₆, Z₇, and Z₈ is a carbon atom to which the remainder of the molecule is attached; or

Z₃ is N; Z₂ and Z₁ are independently CR₂; Z₄ and Z₅ are independently CR₂; Z₆, Z₇, and Z₈ are independently O, S, N, N—R₁ or CR₂; Y₂, Y₃, and Y₁ are C; Y₄ is N; and any one of Z₂, Z₁, Z₆, Z₆, Z₇, and Z₈ is a carbon atom to which the remainder of the molecule is attached; or

Z₁ is N or CR₂; Z₂ is CR₂; Z₃ is O, S, or N—R₁; Z₄ and Z₅ are independently CR₂; Z₆, Z₇, and Z₈ are independently O, S, N, N—R₁, or CR₂; Y₁, Y₂, Y₃, and Y₄ are C; any one of Z₁, Z₂, Z₃, Z₆, Z₇, and Z₈ is a carbon atom to which the remainder of the molecule is attached; and

R₁, R₂, R₄, R₆ and R₇ are as defined above.

In Some Embodiments of Formula 5-A:

Z₁ and Z₃ are independently O, S, N—R₁, N, or CR₂; Z₂ is CR₂; Z₄, Z₅, Z₆, Z₇, Z₈, and Z₉ are independently CR₂; and Y₁, Y₂, Y₃, and Y₄, are C; or

Z₁ and Z₃ are independently O, S, N—R₁, N, or CR₂; Z₂ is CR₂; Z₄ and Z₉ are independently CR₂ or N; Z₅, Z₆, Z₇, and Z₈ are independently CR₂; Y₁, Y₂, Y₃, and Y₄, are C; and any one of Z₁, Z₂, Z₃, Z₅, Z₆, Z₇, and Z₈ is a carbon atom to which the remainder of the molecule is attached; or

Z₁ is S, O, or N—R₁; Z₂, Z₃, Z₄, Z₅, Z₆, Z₇, and Z₈ are independently CR₂; Z₉ is N; and Y₁, Y₂, Y₃, and Y₄, are C; or

Z₁ and Z₃ are independently O, S, N—R₁, N, or CR₂; Z₄ and Z₉ are independently N or CR₂; Z₅, Z₆, Z₇, and Z₈ are independently CR₂ or N; Y₁, Y₂, Y₃, and Y₄ are C; and Z₂ is a carbon to which the remainder of the molecule is attached; or

Z₁ is N; Z₂, Z₃, and Z₄ are independently CR₂; Z₅, Z₆, Z₇, and Z₈ are independently N or CR₂; Z₉ is CR₂ or N; Y₁ is N; Y₂, Y₃, and Y₄, are C; and Z₂ or Z₃ is a carbon to which the remainder of the molecule is attached; or

Z₃ is N; Z₁, Z₂, and Z₄ are independently CR₂; Z₅, Z₆, Z₇, and Z₈ are independently CR₂ or N; Z₉ is CR₂ or N; Y₄ is N; Y₁, Y₂, and Y₃ are C; and

R₁, R₂, R₄, R₆ and R₇ are as defined above.

In Some Embodiments of Formula 5-B:

Z₁ is N; Z₂, Z₃, Z₄, Z₅, Z₆, Z₇, Z₈, and Z₉ are independently CR₂; Y₁ is N; Y₂, Y₃, and Y₄ are C; and any one of Z₂, Z₃, Z₅, Z₇, Z₈, and Z₉ is a carbon to which the remainder of the molecule is attached; or

Z₃ is N; Z₁, Z₂, and Z₄ are independently CR₂; Z₅, Z₆, Z₇, Z₈, and Z₉ are independently CR₂ or N; Y₁, Y₃, and Y₄ are C; and Y₂ is N; or

Z₁ is O, S, or N—R₁; Z₂, Z₃, and Z₄ are independently CR₂; Z₅ is CR₂ or N; Z₆, Z₇, Z₈, and Z₉ are independently CR₂ or N; and any one of Z₆, Z₇, Z₈, and Z₉ is a carbon atom to which the remainder of the molecule is attached; or

Z₁ and Z₃ are independently O, S, N—R₁, N, or CR₂; Z₄ is CR₂ or N; Z₅ is CR₂; Z₆, Z₇, Z₈, and Z₉ are independently CR₂ or N; Y₁ and Y₂ are independently C or N; Y₃ and Y₄ are C; and

R₁, R₂, R₄, R₆ and R₇ are as defined above.

In Some Embodiments of Formula 5-C:

Z₁ and Z₂ are independently N or CR₂; Z₃, Z₄, Z₅, Z₆, Z₇, and Z₈ are independently CR₂; and Y₁ is C; or

Z₁ and Z₂ are independently CR₂; Z₃, Z₄, Z₅, Z₆, Z₇, and Z₈ are independently N or CR₂; and Y₁ is C; or

Z₁, Z₂, Z₃, Z₄, Z₅, Z₆, Z₇, and Z₈ are independently N or CR₂; Y₁ is C; and

R₁, R₂, R₄, R₆ and R₇ are as defined above.

In Some Embodiments of Formula 6-A:

Z₁ is O, S, or N—R₁; Z₂ and Z₃ are independently CR₂; Z₄ is O, S, N—R₁, or CR₂; Y₁ and Y₂ are C; Y₄ and Y₃ are independently C or N; W1, W2, and W₃ are independently CR₄R₄; and t is 1-2; or

Z₁ is O, S, or N—R₁; Z₂ and Z₃ are independently CR₂; Z₄ is O, S, N—R₁, or CR₂; Y₁ and Y₂ are C; Y₄ and Y₃ are independently C or N; W₁ and W₃ are independently CR₄R₄; W₂ is O, S(═O)r where r is 0-2, N—R₁, or CR₄R₄; and t is 1-2; or

Z₃ is N; Z₂ is CR₂; Z₁ is CR₂, or N; Z₄ is O, S, or N—R₁; W₁, W₂, and W₃ are independently CR₄R₄; Y₁, Y₃, and Y₄ are C; Y₂ is N; t is 1-3; and any one of Z₁, Z₂, and Z₄ is a carbon to which the remainder of the molecule is attached; or

Z₁ is N; Z₂ is CR₂; Z₃ is CR₂ or N; Z₄ is O, S, or N—R₁; W₁, W₂ and W₃ are independently CR₄R₄; Y₂, Y₃, and Y₄ are C; Y₁ is N; t is 1-3; and any one of Z₂, Z₃, and Z₄ is a carbon to which the remainder of the molecule is attached; or

Z₃ is N; Z₂ is CR₂; Z₁ is CR₂ or N; Z₄ is O, S, or N—R₁; Y₁, Y₃, and Y₄ are C; Y₂ is N; W₁, W₂, and W₃ are independently CR₄R₄, O, S(═O)r where r is 0-2, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and any one of Z₁, Z₂ and Z₄ is a carbon to which the remainder of the molecule is attached; or

Z₁ is N; Z₂ is CR₂; Z₃ is CR₂, or N; Z₄ is O, S, or N—R₁; Y₂, Y₃, and Y₄ are C; Y₁ is N; W₁, W₂, and W₃ are independently CR₄R₄, O, S(═O)r where r is 0-2, or N—R₁; with the proviso that whenever W₂ is S or O it is not adjacent to a W₁, W₂ or W₃ that is also S or O; t is 1-3; and any one of Z₂, Z₃, and Z₄ is a carbon atom to which the remainder of the molecule is attached; or

Z₁ is CR₂; Z₂ is a carbon to which the remainder of the molecule is attached; Z₃ is N; Z₄ is O, S, or N—R₁; Y₁ is C; Y₂ is N; Y₃ and Y₄ are C; W₁, W₂, and W₃ are independently CR₄R₄, O, S(═O)ᵣ where r is 0-2, or N—R₁; with the proviso that whenever W₂ is S or O it is not adjacent to a W₁, W₂ or W₃ that is also S or O; and t is 1-3; or

Z₁ is a carbon to which the remainder of the molecule is attached; Z₂ is CR₂; Z₃ is N; Z₄ is O, S, or N—R₁; Y₁ is C; Y₂ is N; Y₃ and Y₄ are C; W₁, W₂, and W₃ are independently CR₄R₄, O, S(═O)ᵣ where r is 0-2, or N—R₁; with the proviso that whenever W₂ is S or O it is not adjacent to a W₁, W₂ or W₃ that is also S or O; and t is 1-3; or

Z₁, Z₂, and Z₃ are independently CR₂ or N; Z₄ is CR₂; Y₁ and Y₂ are C; Y₃ and Y₄ are N; W₁, W₂, and W₃ are independently CR₄R₄, O, S(═O)ᵣ where r is 0-2, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and

R₁, R₂, R₄, R₆ and R₇ are as defined above.

In Some Embodiments of Formula 6-B:

Z₁, Z₂, Z₃, and Z₄ are independently CR₂; Y₁ and Y₂ are N; W₁ and W₂ are independently O, S, N—R₁, or CR₄R₄; and t is 1-2; or

Z₁ and Z₂ are independently N or CR₂; Z₃ is CR₂; Z₄ is O, S, or N—R₁; W₁ and W₂ are independently O, S, N—R₁, or CR₄R₄; t is 1-2; and

R₁, R₂, R₄, R₆ and R₇ are as defined above.

In Some Embodiments of Formula 7-A:

Z₁ is O, S, or N—R₁; Z₂, Z₃, Z₄, and Z₅ are independently CR₂; W₁, W₂, and W₃ are independently N—R₁, O, S(═O)ᵣ where r is 0-2, or CR₄R₄; Y₁ and Y₂ are C; t is 1-3; and any one of Z₂ or Z₃ is a carbon to which the remainder of the molecule is attached; or

Z₁ is O, S, or N—R₁; Z₃ is N, O, or S; Z₂, Z₄, and Z₅ are independently CR₂; W₁, W₂, and W₃ are independently N—R₁, O, S(═O)ᵣ where r is 0-2, or CR₄R₄; Y₁ and Y₂ are C; t is 1-3; and Z₂ is a carbon to which the remainder of the molecule is attached; or

Z₁ is CR₂; Z₃ is N; Z₂, Z₄, and Z₅ are independently CR₂; W₁, W₂, and W₃ are independently N—R₁, O, S(═O)ᵣ where r is 0-2, or CR₄R₄; Y₁ is N; Y₂ is C; t is 1-3; and one of Z₁ or Z₂ is a carbon to which the remainder of the molecule is attached; or

Z₁ is N; Z₂, Z₃, Z₄, and Z₅ are independently CR₂; W₁, W₂, and W₃ are independently N—R₁, O, S(═O)ᵣ where r is 0-2, or CR₄R₄; Y₁ is C; Y₂ is N; t is 1-3; and one of Z₂ or Z₃ is a carbon to which the remainder of the molecule is attached; or

Z₁ is O, S, or N—R₁; Z₂ is N, O, or S; Z₃, Z₄, and Z₅ are independently CR₂; W₁, W₂, and W₃ are independently N—R₁, O, S(═O)ᵣ where r is 0-2, or CR₄R₄; Y₁ and Y₂ are C; t is 1-3; and Z₃ is a carbon atom to which the remainder of the molecule is attached; or

Z₁ is O, S, or N—R₁; Z₂ and Z₃ are independently CR₂; Z₄ and Z₅ are independently CR₂ or N; W₁, W₂, and W₃ are independently N—R₁, O, S(═O)ᵣ where r is 0-2, or CR₄R₄; Y₁ and Y₂ are C; t is 1-3; and one of Z₂ or Z₃ is a carbon to which the remainder of the molecule is attached; or

Z₁ is O, S, or N—R₁; Z₃ is N, O, or S; Z₂ is CR₂; Z₄ and Z₅ are independently CR₂ or N; W₁, W₂, and W₃ are independently N—R₁, O, S(═O)ᵣ where r is 0-2, or CR₄R₄; Y₁ and Y₂ are C; t is 1-3; and Z₂ is a carbon to which the remainder of the molecule is attached; or

Z₁ is CR₂; Z₃ is N; Z₂ is CR₂; Z₄ and Z₅ are independently N or CR₂; W₁, W₂, and W₃ are independently N—R₁, O, S(═O)ᵣ where r is 0-2, or CR₄R₄; Y₁ is N; Y₂ is C; t is 1-3; and one of Z₁ or Z₂ is a carbon to which the remainder of the molecule is attached; or

Z₁ is N; Z₂ and Z₃ are independently CR₂; Z₄ and Z₅ are independently N or CR₂; W₁, W₂, and W₃ are independently N—R₁, O, S(═O)ᵣ where r is 0-2, or CR₄R₄; Y₁ is C; Y₂ is N; t is 1-3; and one of Z₂ or Z₃ is a carbon to which the remainder of the molecule is attached; or

Z₁ is O, S, or N—R₁; Z₂ is N, O, or S; Z₃ is CR₂; Z₄ and Z₅ are independently N or CR₂; W₁, W₂, and W₃ are independently N—R₁, O, S(═O)ᵣ where r is 0-2, or CR₄R₄; Y₁ and Y₂ are C; t is 1-3; Z₃ is a carbon to which the remainder of the molecule is attached; and

R₁, R₂, R₄, R₆ and R₇ are as defined above.

In Some Embodiments of Formula 7-B:

Z₁ is O, S, or N—R₁; Z₂, Z₃, Z₄, and Z₅ are independently CR₂; W₁, W₂, and W₃ are independently N—R₁, O, S(═O)ᵣ where r is 0-2, or CR₄R₄; Y₁, Y₂, Y₃, and Y₄ are C; t is 1-3; and one of Z₂ or Z₃ is a carbon to which the remainder of the molecule is attached; or

Z₁ is O, S, or N—R₁; Z₃ is N, O, or S; Z₂, Z₄, and Z₅ are independently CR₂; W₁, W₂, and W₃ are independently N—R₁, O, S(═O)ᵣ where r is 0-2, or CR₄R₄; Y₁, Y₂, Y₃, and Y₄ are C; t is 1-3; and Z₂ is a carbon to which the remainder of the molecule is attached; or

Z₁ is CR₂; Z₃ is N; Z₂, Z₄, and Z₅ are independently CR₂; W₁, W₂, and W₃ are independently N—R₁, O, S(═O)ᵣ where r is 0-2, or CR₄R₄; Y₃ is N; Y₁, Y₂, and Y₄ are C; t is 1-3; and one of Z₁ or Z₂ is a carbon to which the remainder of the molecule is attached; or

Z₁ is N; Z₂, Z₃, Z₄, and Z₅ are independently CR₂; W₁, W₂, and W₃ are independently N—R₁, O, S(═O)ᵣ where r is 0-2, or CR₄R₄; Y₁, Y₂, and Y₃ are C; Y₄ is N; t is 1-3; and one of Z₂ or Z₃ is a carbon to which the remainder of the molecule is attached; or

Z₁ is O, S, or N—R₁; Z₂ is N, O, or S; Z₃, Z₄, and Z₅ are independently CR₂; W₁, W₂, and W₃ are independently N—R₁, O, S(═O)ᵣ where r is 0-2, or CR₄R₄; Y₁, Y₂, Y₃, and Y₄ are C; t is 1-3; and Z₃ is a carbon to which the remainder of the molecule is attached; or

Z₁ is O, S, or N—R₁; Z₂ and Z₃ are independently CR₂; Z₄ and Z₅ are independently CR₂ or N; W₁, W₂, and W₃ are independently N—R₁, O, S(═O)ᵣ where r is 0-2, or CR₄R₄; Y₁, Y₂, Y₃, and Y₄ are C; t is 1-3; and one of Z₂ or Z₃ is a carbon to which the remainder of the molecule is attached; or

Z₁ is O, S, or N—R₁; Z₃ is N, O, or S; Z₂ is CR₂; Z₄ and Z₅ are independently CR₂ or N; W₁, W₂, and W₃ are independently N—R₁, O, S(═O)ᵣ where r is 0-2, or CR₄R₄; Y₁, Y₂, Y₃, and Y₄ are C; t is 1-3; and Z₂ is a carbon to which the remainder of the molecule is attached; or

Z₁ is CR₂; Z₃ is N; Z₂ is CR₂; Z₄ and Z₅ are independently N or CR₂; W₁, W₂, and W₃ are independently N—R₁, O, S(═O)ᵣ where r is 0-2, or CR₄R₄; Y₃ is N; Y₁, Y₂, and Y₄ are C; t is 1-3; and one of Z₁ or Z₂ is a carbon to which the remainder of the molecule is attached; or

Z₁ is N; Z₂ and Z₃ are independently CR₂; Z₄ and Z₅ are independently N or CR₂; W₁, W₂, and W₃ are independently N—R₁, O, S(═O)ᵣ where r is 0-2, or CR₄R₄; Y₁, Y₂, and Y₃ are C; Y₄ is N; t is 1-3; and one of Z₂ or Z₃ is a carbon to which the remainder of the molecule is attached; or

Z₁ is O, S, or N—R₁; Z₂ is N, O, or S; Z₃ is CR₂; Z₄ and Z₅ are independently N or CR₂; W₁, W₂, and W₃ are independently N—R₁, O, S(═O)ᵣ where r is 0-2, or CR₄R₄; Y₁, Y₂, Y₃, and Y₄ are C; t is 1-3; Z₃ is a carbon to which the remainder of the molecule is attached; and

R₁, R₂, R₄, R₆ and R₇ are as defined above.

In Some Embodiments of Formula 7-C:

Z₁, Z₃, Z₄, and Z₅ are independently N or CR₂; Z₂ is O, S, or N—R₁; Y₁ and Y₂ are C; W₁ and W₂ are independently N—R₁, O, S(═O)1 where r is 0-2, or CR₄R₄; and t is 1-2; or

Z₁, Z₃, Z₄, and Z₅ are independently CR₂; Z₂ is O, S, or N—R₁; Y₁and Y₂ are C; W₁ and W₂ are independently N—R₁, O, S(═O)ᵣ where r is 0-2, or CR₄R₄; and t is 1-2; or

Z₁, Z₃, and Z₅ are independently CR₂; Z₂ is O, S, or N—R₁; Z₄ is N; Y₁ and Y₂ are C; W₁ and W₂ are independently N—R₁, O, S(═O)ᵣ where r is 0-2, or CR₄R₄; and t is 1-2; or

Z₁, Z₂, Z₃, Z₄, and Z₅ are independently CR₂; Y₁ is C; Y₂ is N; W₁ and W₂ are independently N—R₁, O, S(═O)ᵣ where r is 0-2, or CR₄R₄; and t is 1-2; or

Z₁, Z₂, Z₃, and Z₅ are independently CR₂; Z₄ is N; Y₁ is C; Y₂ is N; W₁ and W₂ are independently N—R₁, O, S(═O)ᵣ where r is 0-2, or CR₄R₄; t is 1-2; and

R₁, R₂, R₄, R₆ and R₇ are as defined above.

In Some Embodiments of Formula 8-A:

Z₃ and Z₆ are independently O, S, or N—R₁; Z₁, Z₂, Z₄, and Z₅ are independently CR₂; W₁ and W₂ are independently N—R₁, O, S(═O)ᵣ where r is 0-2, or CR₄R₄; and t is 1-2; or

Z₃ and Z₆ are independently O, S, or N—R₁; Z₁ and Z₄ are N; Z₂ and Z₅ are independently CR₂; W₁ and W₂ are independently N—R₁, O, S(═O)ᵣ where r is 0-2, or CR₄R₄; t is 1-2; and Z₂ or Z₅ is a carbon to which the remainder of the molecule is attached; or

Z₁ and Z₄ are independently O, S, or N—R₁; Z₂, Z₃, Z₅, and Z₆ are independently CR₂; W₁ and W₂ are independently N—R₁, O, S(═O)ᵣ where r is 0-2, or CR₄R₄; and t is 1-2.

Z₁ and Z₄ are independently O, S, or N—R₁; Z₃ and Z₆ are N; Z₂ and Z₅ are independently CR₂; W₁ and W₂ are independently N—R₁, O, S(═O)ᵣ where r is 0-2, or CR₄R₄; t is 1-2; and one of Z₂ or Z₅ is a carbon to which the remainder of the molecule is attached; or

Z₂ and Z₅ are independently O, S, or N—R₁; Z₁, Z₃, Z₄, and Z₆ are independently CR₂; W₁ and W₂ are independently N—R₁, O, S(═O)ᵣ where r is 0-2, or CR₄R₄; and t is 1-2; or

Z₂ and Z₅ are independently O, S, or N—R₁; Z₁, Z₃, Z₄, and Z₆ are independently CR₂, N, or S; W₁ and W₂ are independently N—R₁, O, S(═O)ᵣ where r is 0-2, or CR₄R₄; t is 1-2; and any one of Z₁, Z₃, Z₄, and Z₆ is a carbon to which the remainder of the molecule is attached; or

Z₁ is CR₂ or N; Z₂ is CR₂; Z₃ is N; Z₄ and Z₅ are independently CR₂; Z₆ is N; Y₁ and Y₃ are independently CR₂; Y₂ and Y₄ are N; W₁ and W₂ are independently N—R₁, O, S(═O)ᵣ where r is 0-2, or CR₄R₄; t is 1-2; and any one of Z₁, Z₂, Z₄, and Z₅ is a carbon to which the remainder of the molecule is attached; or

Z₁ is CR₂ or N; Z₂ is CR₂; Z₃ is O, S, or N—R₁; Z₄ and Z₅ are independently CR₂; Z₆ is N; Y₁, Y₄, and Y₃ are independently CR₂; Y₂ is N; W₁ and W₂ are independently N—R₁, O, S═(═O)ᵣ where r is 0-2, or CR₄R₄; t is 1-2; and any one of Z₁, Z₂, Z₄, and Z₅ is a carbon to which the remainder of the molecule is attached; or

Z₁ is CR₂ or N; Z₂ is CR₂; Z₃ is N; Z₄, Z₅, and Z₆ are independently N or CR₂; Y₁ and Y₃ are N; Y₂ and Y₄ are independently CR₂; W₁ and W₂ are independently N—R₁, O, S(═O)ᵣ where r is 0-2, or CR₄R₄; t is 1-2; any one of Z₁, Z₂, Z₄, Z₅, and Z₆ is a carbon to which the remainder of the molecule is attached; and

R₁, R₂, R₄, R₆ and R₇ are as defined above.

In Some Embodiments of Formula 8-B:

Z₁, Z₂, Z₄, Z₅, and Z₆ are independently CR₂; Z₃ is O, S, or N—R₁; Y₁, Y₂, and Y₄ are C; Y₃ is N; W₁ and W₂ are independently N—R₁, O, S(═O)ᵣ where r is 0-2, or CR₄R₄; and t is 1-2; or

Z₁, Z₂, Z₄, Z₅, and Z₆ are independently CR₂ or N; Z₃ is O, S, or N—R₁; Y₁, Y₂, and Y₄ are C; Y₃ is N; W₁ and W₂ are independently N—R₁, O, S(═O)ᵣ where r is 0-2, or CR₄R₄; t is 1-2; and any one of Z₁, Z₂, Z₄, Z₅, and Z₆ is a carbon to which the remainder of the molecule is attached; or

Z₁, Z₂, Z₃, Z₅, and Z₆ are independently CR₂; Z₄ is O, S, or N—R₁; Y₁, Y₂, and Y₃ are C; Y₄ is N; W₁ and W₂ are independently N—R₁, O, S(═O)ᵣ where r is 0-2, or CR₄R₄; t is 1-2; and any one of Z₁, Z₂, Z₃, Z₅, any Z₆ is a carbon to which the remainder of the molecule is attached; or

Z₁, Z₂, Z₃, Z₅, and Z₆ are independently CR₂ or N; Z₄ is O, S, or N—R₁; Y₁, Y₂, and Y₃ are C; Y₄ is N; W₁ and W₂ are independently N—R₁, O, S(═O)ᵣ where r is 0-2, or CR₄R₄; t is 1-2; and any one of Z₁, Z₂, Z₃, Z₅, and Z₆ is a carbon to which the remainder of the molecule is attached; or

Z₁, Z₂, Z₃, Z₄, Z₅, and Z₆ are independently CR₂; Y₁ and Y₃ are N; Y₂ and Y₄ are C; W₁ and W₂ are independently N—R₁, O, S(═O)ᵣ where r is 0-2, or CR₄R₄; t is 1-2; and any one of Z₁, Z₂, Z₃, Z₄, Z₅, and Z₆ is a carbon to which the remainder of the molecule is attached; or

Z₁, Z₂, Z₃, Z₄, Z₅, and Z₆ are independently CR₂ or N; Y₁ Y₃ are N; Y₂ and Y₄ are C; W₁ and W₂ are independently N—R₁, O, S═(═O)ᵣ where r is 0-2, or CR₄R₄; t is 1-2; and any one of Z₁, Z₂, Z₃, Z₄, Z₅, and Z₆ is a carbon to which the remainder of the molecule is attached; or

Z₁, Z₃, Z₄, Z₅, and Z₆ are independently CR₂; Z₂ is O, S, or N—R₁; Y₁, Y₂, and Y₄ is C; Y₃ is N; W₁ and W₂ are independently N—R₁, O, S(═O)ᵣ where r is 0-2, or CR₄R₄; t is 1-2; and any one of Z₁, Z₃, Z₄, Z₅, and Z₆ is a carbon to which the remainder of the molecule is attached; or

Z₁, Z₃, Z₄, Z₅, and Z₆ are independently CR₂ or N; Z₂ is O, S, or N—R₁; Y₁, Y₂, and Y₄ are C; Y₃ is N; W₁ and W₂ are independently N—R₁, O, S(═O), where r is 0-2, or CR₄R₄; t is 1-2; and any one of Z₁, Z₃, Z₄, Z₅, and Z₆ is a carbon to which the remainder of the molecule is attached; or

Z₁, Z₂, Z₄, and Z₆ are independently CR₂; Z₃ and Z₅ are independently O, S, or N—R₁; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₄, and Z₆ is a carbon to which the remainder of the molecule is attached; or

Z₁, Z₂, Z₄, and Z₆ are independently CR₂ or N; Z₃ and Z₅ are independently O, S, or N—R₁; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₄, and Z₆ is a carbon to which the remainder of the molecule is attached; or

Z₁, Z₂, Z₄, and Z₅ are independently CR₂; Z₃ and Z₆ are independently O, S, or N—R₁; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₄, and Z₅ is a carbon to which the remainder of the molecule is attached; or

Z₁, Z₂, Z₄, and Z₅ are independently CR₂ or N; Z₃ and Z₆ are independently O, S, or N—R₁; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₄, and Z₅ is a carbon to which the remainder of the molecule is attached; or

Z₁, Z₂, Z₅, and Z₆ are independently CR₂; Z₃ and Z₄ are independently O, S, or N—R₁; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₅, and Z₆ is a carbon to which the remainder of the molecule is attached; or

Z₁, Z₂, Z₅, and Z₆ are independently CR₂ or N; Z₃ and Z₄ are independently O, S, or N—R₁; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₅, and Z₆ is a carbon to which the remainder of the molecule is attached; or

Z₁, Z₂, Z₄, and Z₅ are independently CR₂; Z₃ and Z₆ are independently O, S, or N—R₁; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₅, and Z₆ is a carbon to which the remainder of the molecule is attached; or

Z₁, Z₂, Z₄, and Z₅ are independently CR₂ or N; Z₃ and Z₆ are independently O, S, or N—R₁; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₅, and Z₆ is a carbon to which the remainder of the molecule is attached; or

Z₁, Z₂, Z₃, Z₄, and Z₆ are independently CR₂; Z₅ is O, S, or N—R₁; Y₁ is N; Y₂, Y₃, and Y₄ are C; W₁ and W₂ are independently N—R₁, O, S═(═O)ᵣ where r is 0-2, or CR₄R₄; t is 1-2; and any one of Z₁, Z₂, Z₃, Z₄, and Z₆ is a carbon to which the remainder of the molecule is attached; or

Z₁, Z₂, Z₃, Z₄, and Z₆ are independently CR₂ or N; Z₅ is O, S, or N—R₁; Y₁ is N; Y₂, Y₃, and Y₄ are C; W₁ and W₂ are independently N—R₁, O, where r is 0-2, or CR₄R₄; t is 1-2; and any one of Z₁, Z₂, Z₃, Z₄, and Z₆ is a carbon to which the remainder of the molecule is attached; or

Z₁, Z₃, Z₄, and Z₆ are independently CR₂; Z₂ and Z₅ are independently O, S, or N—R₁; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₃, Z₄, and Z₆ is a carbon to which the remainder of the molecule is attached; or

Z₁, Z₃, Z₄, and Z₆ are independently CR₂ or N; Z₂ and Z₅ are independently O, S, or N—R₁; Y₁, Y₂, Y₃, and Y₄ are C; W₁ and W₂ are independently N—R₁, O, S(═O)ᵣ where r is 0-2, or CR₄R₄; t is 1-2; any one of Z₁, Z₃, Z₄, and Z₆ is a carbon to which the remainder of the molecule is attached; and

R₁, R₂, R₄, R₆ and R₇ are as defined above.

In Some Embodiments of Formula 9-A:

Z₁ is O, S, or N—R₁; Z₂, Z₄, Z₅, Z₆, and Z₇ are independently CR₂; Z₃ is is CR₂, or N; Y₁, Y₂, Y₃, and Y₄ are C; W₁ and W₂ are independently N—R₁, O, S(═O)ᵣ where r is 0-2, or CR₄R₄; t is 1-2; and any one of Z₂, Z₃, Z₄, and Z₆ is a carbon to which the remainder of the molecule is attached; or

Z₁ is O, S, or N—R₁; Z₂, Z₄, Z₅, Z₆, and Z₇ are independently CR₂; Z₃ is CR₂; Y₁, Y₂, Y₃, and Y₄ are C; W₁ and W₂ are independently N—R₁, O, S(═O)ᵣ where r is 0-2, or CR₄R₄; t is 1-2; and any one of Z₂, Z₃, Z₄, Z₅, Z₆, and Z₇ is a carbon to which the remainder of the molecule is attached; or

Z₁ is O, S, or N—R₁; Z₂, Z₄, Z₅, Z₆, and Z₇ are independently CR₂; Z₃ is N; Y₁, Y₂, Y₃, and Y₄ are C; W₁ and W₂ are independently N—R₁, O, S(═O)ᵣ where r is 0-2, or CR₄R₄; t is 1-2; and any one of Z₂, Z₄, Z₅, Z₆, and Z₇ is a carbon to which the remainder of the molecule is attached; or

Z₁ is O, S, or N—R₁; Z₃, Z₄, Z₅, Z₆, and Z₇ are independently CR₂; Z₂ is CR₂ or N; Y₁, Y₂, Y₃, and Y₄ are C; W₁ and W₂ are independently N—R₁, O, S(═O)ᵣ where r is 0-2, or CR₄R₄; t is 1-2; and any one of Z₂, Z₃, Z₄, Z₅, Z₆, and Z₇ is a carbon to which the remainder of the molecule is attached; or

Z₁ is O, S, or N—R₁; Z₃, Z₄, Z₅, Z₆, and Z₇ are independently CR₂; Z₂ is N; Y₁, Y₂, Y₃, and Y₄ are C; W₁ and W₂ are independently N—R₁, O, S(═O)ᵣ where r is 0-2, or CR₄R₄; t is 1-2; and any one of Z₃, Z₄, Z₅, Z₆, and Z₇ is a carbon to which the remainder of the molecule is attached; or

Z₁ is O, S, or N—R₁; Z₃, Z₄, Z₅, Z₆, and Z₇ are independently CR₂; Z₂ is CR₂; Y₁, Y₂, Y₃, and Y₄ are C; W₁ and W₂ are independently N—R₁, O S(═O)ᵣ where r is 0-2, or CR₄R₄; t is 1-2; and any one of Z₂, Z₃, Z₄, Z₅, Z₆, and Z₇ is a carbon to which the remainder of the molecule is attached; or

Z₁ is O, S, or N—R₁; Z₂, Z₃, Z₄, Z₅, Z₆, and Z₇ are independently N or CR₂; Y₁, Y₂, Y₃, and Y₄ are C; W₁ and W₂ are independently N—R₁, O, S(═O)ᵣ where r is 0-2, or CR₄R₄; t is 1-2; and any one of Z₂, Z₃, Z₄, Z₅, Z₆, and Z₇ is a carbon to which the remainder of the molecule is attached; or

Z₁ is O, S, or N—R₁; Z₂ is CR₂; Z₄, Z₅, Z₆, and Z₇ are independently N or CR₂; Z₃ is CR₂; Y₁, Y₂, Y₃, and Y₄ are C; W₁ and W₂ are independently N—R₁, O, S(═O)ᵣ where r is 0-2, or CR₄R₄; t is 1-2; and any one of Z₂, Z₃, Z₄, Z₅, Z₆, and Z₇ is a carbon to which the remainder of the molecule is attached; or

Z₁ is O, S, or N—R₁; Z₂ is CR₂; Z₄, Z₅, Z₆, and Z₇ are independently N or CR₂; Z₃ is N; Y₁, Y₂, Y₃, and Y₄ are C; W₁ and W₂ are independently N—R₁, O, S(═O)ᵣ where r is 0-2, or CR₄R₄; t is 1-2; and any one of Z₂, Z₄, Z₅, Z₆, and Z₇ is a carbon to which the remainder of the molecule is attached; or

Z₁ is O, S, or N—R₁; Z₄, Z₅, Z₆, and Z₇ are independently N or CR₂; Z₂ and Z₃ are independently CR₂ or N; Y₁, Y₂, Y₃, and Y₄ are C; W₁ and W₂ are independently N—R₁, O, S(═O)ᵣ where r is 0-2, or CR₄R₄; t is 1-2; and any one of Z₂, Z₃, Z₄, Z₅, Z₆, and Z₇ is a carbon to which the remainder of the molecule is attached; or

Z₁ is O, S, or N—R₁; Z₃ is CR₂; Z₄, Z₅, Z₆, and Z₇ are independently N or CR₂; Z₂ is N; Y₁, Y₂, Y₃, and Y₄ are C; W₁ and W₂ are independently N—R₁, O, S(═O)ᵣ where r is 0-2, or CR₄R₄; t is 1-2; and any one of Z₃, Z₄, Z₅, Z₆, and Z₇ is a carbon to which the remainder of the molecule is attached; or

Z₁ is O, S, N—R₁; Z₄, Z₅, Z₆, and Z₇ are independently N or CR₂; Z₂ and Z₃ are independently CR₂; Y₁, Y₂, Y₃, and Y₄ are C; W₁ and W₂ are independently N—R₁, O, S(═O)ᵣ where r is 0-2, or CR₄R₄; t is 1-2; and any one of Z₂, Z₃, Z₄, Z₅, Z₆, and Z₇ is a carbon to which the remainder of the molecule is attached; or

Z₃ is O, S, or N—R₁, Z₂, Z₄, Z₅, Z₆, and Z₇ are independently CR₂; Z₁ is CR₂ or N; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₄, Z₅, Z₆, and Z₇ is a carbon to which the remainder of the molecule is attached; or

Z₃ is O, S, or N—R₁; Z₁, Z₂, Z₄, Z₅, Z₆, and Z₇ are independently CR₂; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₄, Z₅, Z₆, and Z₇ is a carbon to which the remainder of the molecule is attached.

Z₃ is O, S, or N—R₁; Z₂, Z₄, Z₅, Z₆, and Z₇ are independently CR₂; Z₁ is N; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₄, Z₅, Z₆, and Z₇ is a carbon to which the remainder of the molecule is attached; or

Z₃ is O, S, or N—R₁; Z₁, Z₄, Z₅, Z₆, and Z₇ are independently CR₂; Z₂ is CR₂ or N; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₄, Z₅, Z₆, and Z₇ is a carbon to which the remainder of the molecule is attached; or

Z₃ is O, S, or N—R₁; Z₁, Z₄, Z₅, Z₆, and Z₇ are independently CR₂; Z₂ is N; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₄, Z₅, Z₆, and Z₇ is a carbon to which the remainder of the molecule is attached; or

Z₃ is O, S, or N—R₁; Z₁, Z₂, Z₄, Z₅, Z₆, and Z₇ are independently CR₂; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₄, Z₅, Z₆, and Z₇ is a carbon to which the remainder of the molecule is attached; or

Z₃ is O, S, or N—R₁; Z₂, Z₄, Z₅, Z₆, and Z₇ are independently N or CR₂; Z₁ is CR₂ or N; Y₁, Y₂, Y₃, and Y₄ are C; W₁ and W₂ are independently N—R₁, O, S(═O)ᵣ where r is 0-2, CR₄R₄; t is 1-2; and any one of Z₁, Z₂, Z₄, Z₅, Z₆, and Z₇ is a carbon to which the remainder of the molecule is attached; or

Z₃ is O, S, or N—R₁; Z₂ is CR₂; Z₄, Z₅, Z₆, and Z₇ are independently N or CR₂; Z₁ is CR₂; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₄, Z₅, Z₆, and Z₇ is a carbon to which the remainder of the molecule is attached; or

Z₃ is O, S, or N—R₁; Z₂ is CR₂; Z₄, Z₅, Z₆, and Z₇ are independently N or CR₂; Z₁ is N; Y₁, Y₂, Y₃, and Y₄ are C; W₁ and W₂ are independently N—R₁, O, S(═O)ᵣ where r is 0-2, or CR₄R₄; t is 1-2; and any one of Z₂, Z₄, Z₅, Z₆, and Z₇ is a carbon to which the remainder of the molecule is attached; or

Z₃ is O, S, or N—R₁; Z₄, Z₅, Z₆, and Z₇ are independently N or CR₂; Z₂ and Z₁ are independently CR₂ or N; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₄, Z₅, Z₆, and Z₇ is a carbon to which the remainder of the molecule is attached; or

Z₃ is O, S, N—R₁; Z₁ is CR₂; Z₄, Z₅, Z₆, and Z₇ are independently N or CR₂; Z₂ is N; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₄, Z₅, Z₆, and Z₇ is a carbon to which the remainder of the molecule is attached; or

Z₃ is O, S, or N—R₁; Z₄, Z₅, Z₆, and Z₇ are independently N or CR₂; Z₁ and Z₂ are independently CR₂; Y₁, Y₂, Y₃, and Y₄ are C; W₁ and W₂ are independently N—R₁, O, S(═O)ᵣ where r is 0-2, or CR₄R₄; t is 1-2; and any one of Z₁, Z₂, Z₄, Z₅, Z₆, and Z₇ is a carbon to which the remainder of the molecule is attached; or

Z₁, Z₂, Z₃, Z₄, Z₅, Z₆, and Z₇ are independently CR₂; Y₄ is N; Y₁, Y₂, and Y₃ are C; W₁ and W₂ are independently N—R₁, O, S(═O)ᵣ where r is 0-2, or CR₄R₄; t is 1-2; and any one of Z₁, Z₂, Z₃, Z₄, Z₅, Z₆, and Z₇ is a carbon to which the remainder of the molecule is attached; or

Z₁ is N; Z₂, Z₃, Z₄, Z₅, Z₆, and Z₇ are independently CR₂; Y₄ is N; Y₁, Y₂, and Y₃ are C; W₁ and W₂ are independently N—R₁, O, S(═O)ᵣ where r is 0-2, or CR₄R₄; t is 1-2; and any one of Z₂, Z₃, Z₄, Z₅, Z₆, and Z₇ is a carbon to which the remainder of the molecule is attached; or

Z₂ is N; Z₁, Z₃, Z₄, Z₅, Z₆, and Z₇ are independently CR₂; Y₄ is N; Y₁, Y₂, and Y₃ are C; W₁ and W₂ are independently N—R₁, O, S(═O)ᵣ where r is 0-2, or CR₄R₄; t is 1-2; and any one of Z₁, Z₃, Z₄, Z₅, Z₆, and Z₇ is a carbon to which the remainder of the molecule is attached; or

Z₃ is N; Z₁, Z₂, Z₄, Z₅, Z₆, and Z₇ are independently CR₂; Y₄ is N; Y₁, Y₂, and Y₃ are C; W₁ and W₂ are independently N—R₁, O, S(═O)ᵣ where r is 0-2, or CR₄R₄; t is 1-2; and any one of Z₁, Z₂, Z₄, Z₅, Z₆, and Z₇ is a carbon to which the remainder of the molecule is attached; or

Z₁ and Z₂ are N; Z₃, Z₄, Z₅, Z₆, and Z₇ are independently CR₂; Y₄ is N; Y₁, Y₂, and Y₃ are C; W₁ and W₂ are independently N—R₁, O, S(═O)ᵣ where r is 0-2, or CR₄R₄; t is 1-2; and any one of Z₃, Z₄, Z₅, Z₆, and Z₇ is a carbon to which the remainder of the molecule is attached; or

Z₁ and Z₃ are N; Z₂, Z₄, Z₅, Z₆, and Z₇ are independently CR₂; Y₄ is N; Y₁, Y₂, and Y₃ are C; W₁ and W₂ are independently N—R₁, O, S(═O), where r is 0-2, or CR₄R₄; t is 1-2; and any one of Z₁, Z₂, Z₃, Z₄, Z₅, Z₅, and Z₇ is a carbon to which the remainder of the molecule is attached; or

Z₁, Z₂, and Z₃ are N; Z₄, Z₅, Z₆, and Z₇ are independently CR₂; Y₄ is N; Y₁, Y₂, and Y₃ are C; W₁ and W₂ are independently N—R₁, O, S(═O)ᵣ where r is 0-2, or CR₄R₄; t is 1-2; and any one of Z₄, Z₅, Z₆, and Z₇ is a carbon to which the remainder of the molecule is attached; or

Z₁, Z₂, and Z₃ are independently CR₂; Z₄, Z₅, Z₆, and Z₇ are independently N or CR₂; Y₄ is N; Y₁, Y₂, and Y₃ are C; W₁ and W₂ are independently N—R₁, O, S(═O)ᵣ where r is 0-2, or CR₄R₄; t is 1-2; and any one of Z₁, Z₂, Z₃, Z₄, Z₅, Z₆, and Z₇ is a carbon to which the remainder of the molecule is attached; or

Z₁ is N; Z₂ and Z₃ are independently CR₂; Z₄, Z₅, Z₆, and Z₇ are independently N or CR₂; Y₄ is N; Y₁, Y₂, and Y₃ are C; W₁ and W₂ are independently N—R₁, O, S(═O)ᵣ where r is 0-2, or CR₄R₄; t is 1-2; and any one of Z₂, Z₃, Z₄, Z₅, Z₆, and Z₇ is a carbon to which the remainder of the molecule is attached; or

Z₂ is N; Z₁ and Z₃ are independently CR₂; Z₄, Z₅, Z₆, and Z₇ are independently N or CR₂; Y₄ is N; Y₁, Y₂, and Y₃ are C; W₁ and W₂ are independently N—R₁, O, S(═O)ᵣ where r is 0-2, or CR₄R₄; t is 1-2; and any one of Z₁, Z₃, Z₄, Z₅, Z₆, and Z₇ is a carbon to which the remainder of the molecule is attached; or

Z₃ is N; Z₁ and Z₂ are independently CR₂; Z₄, Z₅, Z₆, and Z₇ are independently N or CR₂; Y₄ is N; Y₁, Y₂, and Y₃ are C; W₁ and W₂ are independently N—R₁, O, S(═O)ᵣ where r is 0-2, or CR₄R₄; t is 1-2; and any one of Z₁, Z₂, Z₄, Z₅, Z₆, and Z₇ is a carbon to which the remainder of the molecule is attached; or

Z₁ and Z₂ are N; Z₃ are independently CR₂; Z₄, Z₅, Z₆, and Z₇ are independently N or CR₂; Y₄ is N; Y₁, Y₂, and Y₃ are C; W₁ and W₂ are independently N—R₁, O, S(═O)ᵣ where r is 0-2, or CR₄R₄; t is 1-2; and any one of Z₃, Z₄, Z₅, Z₆, and Z₇ is a carbon to which the remainder of the molecule is attached; or

Z₁ and Z₃ are N; Z₂ are independently CR₂; Z₄, Z₅, Z₆, and Z₇ are independently N or CR₂; Y₄ is N; Y₁, Y₂, and Y₃ are C; W₁ and W₂ are independently N—R₁, O, S(═O)ᵣ where r is 0-2, or CR₄R₄; t is 1-2; and any one of Z₁, Z₂, Z₃, Z₄, Z₅, Z₆, and Z₇ is a carbon to which the remainder of the molecule is attached; or

Z₁, Z₂, and Z₃ are N; Z₄, Z₅, Z₆, and Z₇ are independently N or CR₂; Y₄ is N; Y₁, Y₂, and Y₃ are C; W₁ and W₂ are independently N—R₁, O, S(═O)ᵣ where r is 0-2, or CR₄R₄; t is 1-2; any one of Z₄, Z₅, Z₆, and Z₇ is a carbon to which the remainder of the molecule is attached; and

R₁, R₂, R₄, R₆ and R₇ are as defined above.

In Some Embodiments of Formula 9-B:

Z₁ is O, S, or N—R₁; Z₂, Z₃, Z₄, Z₅, Z₆, and Z₇ are independently N or CR₂; Y₁, Y₂, Y₃, and Y₄ are C; W₁ and W₂ are independently N—R₁, O, S(═O)ᵣ where r is 0-2, or CR₄R₄; t is 0-2; and any one of Z₂, Z₃, Z₄, Z₅, Z₆, and Z₇ is a carbon to which the remainder of the molecule is attached; or

Z₁ is O, S, or N—R₁; Z₂, Z₃, Z₄, Z₅, Z₆, and Z₇ are CH₂; Y₁, Y₂, Y₃, and Y₄ are C; W₁ and W₂ are independently N—R₁, O, S(═O)ᵣ where r is 0-2, or CR₄R₄; t is 0-2; and any one of Z₂, Z₃, Z₄, Z₅, Z₆, and Z₇ is a carbon to which the remainder of the molecule is attached; or

Z₁ is O, S, or N—R₁; Z₂ is N; Z₃, Z₄, Z₅, Z₆, and Z₇ are independently N or CR₂; Y₁, Y₂, Y₃, and Y₄ are C; W₁ and W₂ are independently N—R₁, O, S(═O)ᵣ where r is 0-2, or CR₄R₄; t is 0-2; and any one of Z₃, Z₄, Z₅, Z₆, and Z₇ is a carbon to which the remainder of the molecule is attached; or

Z₁ is O, S, or N—R₁; Z₂ and Z₃ are N; Z₄, Z₅, Z₆, and Z₇ are independently N or CR₂; Y₁, Y₂, Y₃, and Y₄ are C; W₁ and W₂ are independently N—R₁, O, S(═O)ᵣ where r is 0-2, or CR₄R₄; t is 0-2; and any one of Z₄, Z₅, Z₆, and Z₇ is a carbon to which the remainder of the molecule is attached; or

Z₁ is O, S, or N—R₁; Z₂, Z₃, Z₄, Z₅, Z₆, and Z₇ are independently N or CR₂; Y₁, Y₂, Y₃, and Y₄ are C; W₁ and W₂ are independently N—R₁, O, S(═O)ᵣ where r is 0-2, or CR₄R₄; t is 0-2; Z₃ is N; and any one of Z₂, Z₄, Z₅, Z₆, and Z₇ is a carbon to which the remainder of the molecule is attached; or

Z₂ is O, S, or N—R₁; Z₁, Z₃, Z₄, Z₅, Z₆, and Z₇ are independently N or CR₂; Y₁, Y₂, Y₃, and Y₄ are C; W₁ and W₂ are independently N—R₁, O, S(═O)ᵣ where r is 0-2, or CR₄R₄; t is 0-2; and any one of Z₁, Z₃, Z₄, Z₅, Z₆, and Z₇ is a carbon to which the remainder of the molecule is attached; or

Z₂ is O, S, or N—R₁; Z₁, Z₃, Z₄, Z₅, Z₆, and Z₇are independently CR₂; Y₁, Y₂, Y₃, and Y₄ are C; W₁ and W₂ are independently N—R₁, O, S(═O)ᵣ where r is 0-2, or CR₄R₄; t is 0-2; and any one of Z₁, Z₃, Z₄, Z₅, Z₆, and Z₇ is a carbon to which the remainder of the molecule is attached; or

Z₂ is O, S, or N—R₁; Z₁ is N; Z₃ is CR₂; Z₄, Z₅, Z₆, and Z₇ are independently N, CR₂; Y₁, Y₂, Y₃, and Y₄ are C; W₁ and W₂ are independently N—R₁, O, S(═O)ᵣ where r is 0-2, or CR₄R₄; t is 0-2; and any one of Z₃, Z₄, Z₅, Z₆, and Z₇ is a carbon to which the remainder of the molecule is attached; or

Z₂ is O, S, or N—R₁; Z₁ and Z₃ are N; Z₄, Z₅, Z₆, and Z₇ are independently N or CR₂; Y₁, Y₂, Y₃, and Y₄ are C; W₁ and W₂ are independently N—R₁, O, S(═O)ᵣ where r is 0-2, or CR₄R₄; t is 0-2; and any one of Z₄, Z₅, Z₆, and Z₇ is a carbon to which the remainder of the molecule is attached; or

Z₃ is O, S, or N—R₁; Z₁, Z₂, Z₄, Z₅, Z₆, and Z₇ are independently N and CR₂; Y₁, Y₂, Y₃, and Y₄ are C; W₁ and W₂ are independently N—R₁, O, S(═O)ᵣ where r is 0-2, or CR₄R₄; t is 0-2; and any one of Z₁, Z₂, Z₄, Z₅, Z₆, and Z₇ is a carbon to which the remainder of the molecule is attached; or

Z₃ is O, S, or N—R₁; Z₁, Z₂, Z₄, Z₅, Z₆ and Z₇ are independently CR₂; Y₁, Y₂, Y₃, and Y₄ are C; W₁ and W₂ are independently N—R₁, O, S(═O)ᵣ where r is 0-2, or CR₄R₄; t is 0-2; and any one of Z₁, Z₂, Z₄, Z₅, Z₅, and Z₇ is a carbon to which the remainder of the molecule is attached; or

Z₃ is O, S, or N—R₁; Z₁ is N; Z₂ is CR₂, Z₄, Z₅, Z₆ and Z₇ are independently N or CR₂; Y₁, Y₂, Y₃, and Y₄ are C; W₁ and W₂ are independently N—R₁, O, S(═O)ᵣ where r is 0-2, or CR₄R₄; t is 0-2; and any one of Z₂, Z₄, Z₅, Z₆ and Z₇ is a carbon to which the remainder of the molecule is attached; or

Z₃ is O, S, or N—R₁; Z₁ and Z₂ is N; Z₄, Z₅, Z₆, and Z₇ are independently N or CR₂; Y₁, Y₂, Y₃, and Y₄ are C; W₁ and W₂ are independently N—R₁, O, S(═O)ᵣ where r is 0-2, or CR₄R₄; t is 0-2; and any one of Z₄, Z₅, Z₆ and Z₇ is a carbon to which the remainder of the molecule is attached; or

Z₁, Z₂, Z₃, Z₄, Z₅, Z₆, and Z₇ are independently N or CR₂; Y₁ is N; Y₂, Y₃, and Y₄ are C; W₁ and W₂ are independently N—R₁, O, S(═O)ᵣ where r is 0-2, or CR₄R₄; t is 0-2; and any one of Z₁, Z₂, Z₃, Z₄, Z₅, Z₆, and Z₇ is a carbon to which the remainder of the molecule is attached; or

Z₁ is N; Z₂ and Z₃ are independently CR₂; Z₄, Z₅, Z₆ and Z₇ are independently N or CR₂; Y₁ is N; Y₂, Y₃, and Y₄ are C; W₁ and W₂ are independently N—R₁, O, S(═O)ᵣ where r is 0-2, or CR₄R₄; t is 0-2; and any one of Z₂, Z₃, Z₄, Z₅, Z₆ and Z₇ is a carbon to which the remainder of the molecule is attached; or

Z₁ and Z₂ are N; Z₃ is CR₂; Z₄, Z₅, Z₆, and Z₇ are independently N or CR₂; Y₁ is N; Y₂, Y₃, and Y₄ are C; W₁ and W₂ are independently N—R₁, O, S(═O)ᵣ where r is 0-2, or CR₄R₄; t is 0-2; and any one of Z₃, Z₄, Z₅, Z₆, and Z₇ is a carbon to which the remainder of the molecule is attached; or

Z₁, Z₂, and Z₃ are N; Z₄, Z₅, Z₆, and Z₇ are independently N or CR₂; Y₁ is N; Y₂, Y₃, and Y₄ are C; W₁, and W₂ are independently N—R₁, O, S(═O)ᵣ where r is 0-2, or CR₄R₄; t is 0-2; and any one of Z₄, Z₅, Z₆, and Z₇ is a carbon to which the remainder of the molecule is attached; or

Z₂ is N; Z₁ and Z₃ are independently CR₂; Z₄, Z₅, Z₆, and Z₇ are independently N or CR₂; Y₁ is N; Y₂, Y₃, and Y₄ are C; W₁ and W₂ are independently N—R₁, O, S(═O)ᵣ where r is 0-2, or CR₄R₄; t is 0-2; and any one of Z₃, Z₄, Z₅, Z₆, and Z₇ is a carbon to which the remainder of the molecule is attached; or

Z₂ and Z₃ are N; Z₁ is CR₂; Z₄, Z₅, Z₆, and Z₇ are independently N or CR₂; Y₁ is N; Y₂, Y₃, and Y₄ are C; W₁ and W₂ are independently N—R₁, O, S(═O)ᵣ where r is 0-2, or CR₄R₄; t is 0-2; and any one of Z₁, Z₄, Z₅, Z₆, and Z₇ is a carbon to which the remainder of the molecule is attached; or

Z₃ is N; Z₁ and Z₂ are independently CR₂; Z₄, Z₅, Z₆, and Z₇ are independently N or CR₂; Y₁ is N; Y₂, Y₃, and Y₄ are C; W₁ and W₂ are independently N—R₁, O, S(═O)ᵣ where r is 0-2, or CR₄R₄; t is 0-2; and any one of Z₇, Z₂, Z₄, Z₅, Z₆, and Z₇ is a carbon to which the remainder of the molecule is attached; or

Z₁, Z₂, Z₃, Z₄, Z₅, Z₆, and Z₇ are independently N or CR₂; Y₄ is N; Y₂, Y₃, and Y₁ are C; W₁ and W₂ are independently N—R₁, O, S(═O)ᵣ where r is 0-2, or CR₄R₄; t is 0-2; and any one of Z₁, Z₂, Z₃, Z₄, Z₅, Z₆, and Z₇ is a carbon to which the remainder of the molecule is attached; or

Z₁ is N; Z₂ and Z₃ are independently CR₂; Z₄, Z₅, Z₆, and Z₇ are independently N or CR₂; Y₄ is N, Y₂, Y₃, and Y₁ are C; W₁ and W₂ are independently N—R₁, O, S(═O)ᵣ where r is 0-2, or CR₄R₄; t is 0-2; and any one of Z₂, Z₃, Z₄, Z₅, Z₆, and Z₇ is a carbon to which the remainder of the molecule is attached; or

Z₁ and Z₂ are N; Z₃ is CR₂; Z₄, Z₅, Z₆, and Z₇ are independently N or CR₂; Y₄ is N; Y₂, Y₃, and Y₁ are C; W₁ and W₂ are independently N—R₁, O, S(═O)ᵣ where r is 0-2, or CR₄R₄; t is 0-2; and any one of Z₃, Z₄, Z₅, Z₆, and Z₇ is a carbon to which the remainder of the molecule is attached; or

Z₁, Z₂, and Z₃ are N; Z₄, Z₅, Z₆, and Z₇ are independently N or CR₂; Y₄ is N; Y₂, Y₃, and Y₁ are C; W₁ and W₂ are independently N—R₁, O, S(═O)ᵣ where r is 0-2, or CR₄R₄; t is 0-2; and any one of Z₄, Z₅, Z₆, and Z₇ is a carbon to which the remainder of the molecule is attached; or

Z₂ is N; Z₁ and Z₃ are independently CR₂, Z₄, Z₅, Z₆, and Z₇ are independently N or CR₂; Y₄ is N; Y₂, Y₃, and Y₁ are C; W₁ and W₂ are independently N—R₁, O, S(═O)ᵣ where r is 0-2, or CR₄R₄; t is 0-2; and any one of Z₁, Z₃, Z₄, Z₅, Z₆, and Z₇ is a carbon to which the remainder of the molecule is attached; or

Z₂ and Z₃ are N; Z₁ is CR₂; Z₄, Z₅, Z₆, and Z₇ are independently N or CR₂; Y₄ is N; Y₂, Y₃, and Y₁ are C; W₁ and W₂ are independently N—R₁, O, S(═O)ᵣ where r is 0-2, or CR₄R₄; t is 0-2; and any one of Z₁, Z₄, Z₅, Z₆, and Z₇ is a carbon to which the remainder of the molecule is attached; or

Z₃ is N; Z₁ and Z₂ are independently CR₂; Z₄, Z₅, Z₆, and Z₇ are independently N or CR₂; Y₄ is N; Y₂, Y₃, and Y₁ are C; W₁ and W₂ are independently N—R₁, O, S(═O)ᵣ where r is 0-2, or CR₄R₄; t is 0-2; any one of Z₁, Z₂, Z₄, Z₅, Z₆, and Z₇ is a carbon to which the remainder of the molecule is attached; and

R₁, R₂, R₄, R₆ and R₇ are as defined above.

In Some Embodiments of Formula 10-A:

Z₁ is O, S, or N—R₁; Z₂ and Z₃ are independently CR₂; Y₁ and Y₄ are C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and one of Z₂ or Z₃ is a carbon to which the remainder of the molecule is attached; or

Z₁ is O, S, or N—R₁; Z₂ is N; Z₃ is CR₂; Y₁ and Y₄ are C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and Z₃ is a carbon to which the remainder of the molecule is attached; or

Z₁ is O, S, or N—R₁; Z₃ is N; Z₂ is CR₂; Y₁ and Y₄ are C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and Z₂ is a carbon to which the remainder of the molecule is attached; or

Z₂ is O, S, or N—R₁; Z₁ and Z₃ are independently CR₂; Y₁ and Y₄ are C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and one of Z₂ or Z₃ is a carbon to which the remainder of the molecule is attached; or

Z₂ is O, S, or N—R₁; Z₁ is N; Z₃ is CR₂; Y₁ and Y₄ are C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R_{4;} t is 0-2; u is 1-3; and Z₃ is a carbon to which the remainder of the molecule is attached; or

Z₂ is O, S, or N—R₁; Z₃ is N; Z₁ is CR₂; Y₁ and Y₄ are C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and Z₁ is a carbon to which the remainder of the molecule is attached; or

Z₃ is O, S, or N—R₁; Z₁ and Z₂ are independently CR₂; Y₁ and Y₄ are C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and one of Z₁ or Z₂ is a carbon to which the remainder of the molecule is attached; or

Z₃ is O, S, or N—R₁; Z₁ is N; Z₂ is CR₂; Y₁ and Y₄ are C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and Z₂ is a carbon to which the remainder of the molecule is attached; or

Z₃ is O, S, or N—R₁; Z₁ is CH₂; Z₂ is N; Y₁ and Y₄ are C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and Z₁ is a carbon to which the remainder of the molecule is attached; or

Z₁, Z₂, and Z₃ are independently CR₂; Y₁ is N; Y₄ is C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and any one of Z₁, Z₂, and Z₃ is a carbon to which the remainder of the molecule is attached; or

Z₁ is N; Z₂ and Z₃ are independently CR₂; Y₁ is N; Y₄ is C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and one of Z₂ or Z₃ is a carbon to which the remainder of the molecule is attached; or

Z₁ and Z₂ is N; Z₃ is CR₂; Y₁ is N; Y₄ is C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and Z₃ is a carbon to which the remainder of the molecule is attached; or

Z₁ and Z₃ are N; Z₂ is CR₂; Y₁ is N; Y₄ is C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and Z₂ is a carbon to which the remainder of the molecule is attached; or

Z₁, Z₂, and Z₃ are independently CR₂; Y₄ is N; Y₁ is C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and any one of Z₁, Z₂, and Z₃ is a carbon to which the remainder of the molecule is attached; or

Z₁ is N; Z₂ and Z₃ are independently CR₂; Y₄ is N; Y₁ is C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and one of Z₂ or Z₃ is a carbon to which the remainder of the molecule is attached; or

Z₁ and Z₂ are N; Z₃ is CR₂; Y₄ is N; Y₁ is C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and Z₃ is a carbon to which the remainder of the molecule is attached; or

Z₁ and Z₃ are N; Z₂ is CR₂; Y₄ is N; Y₁ is C; Y₂ and Y₃ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; Z₂ is a carbon to which the remainder of the molecule is attached; and

R₁, R₂, R₄, R₆ and R₇ are as defined above.

In Some Embodiments of Formula 10-B:

Z₁ is O, S, or N—R₁; Z₂ and Z₃ are independently CR₂; Y₁ and Y₂ are C; Y₃ and Y₄ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and one of Z₂ or Z₃ is a carbon to which the remainder of the molecule is attached; or

Z₁ is O, S, or N—R₁; Z₂ is N; Z₃ is CR₂; Y₁ and Y₂ are C; Y₃ and Y₄ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; Z₃ is a carbon to which the remainder of the molecule is attached; or

Z₁ is O, S, or N—R₁; Z₃ is N; Z₂ is CR₂; Y₁ and Y₂ are C; Y₃ and Y₄ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and Z₂ is a carbon to which the remainder of the molecule is attached; or

Z₂ is O, S, or N—R₁; Z₁ and Z₃ are independently CR₂; Y₁ and Y₂ are C; Y₃ and Y₄ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and one of Z₂ or Z₃ is a carbon to which the remainder of the molecule is attached; or

Z₂ is O, S, or N—R₁; Z₁ is N; Z₃ is CR₂; Y₁ and Y₂ are C; Y₃ and Y₄ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and Z₃ is a carbon to which the remainder of the molecule is attached; or

Z₂ is O, S, or N—R₁; Z₃ is N; Z₁ is CR₂; Y₁ and Y₂ are C; Y₃ and Y₄ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and Z₁ is a carbon to which the remainder of the molecule is attached; or

Z₃ is O, S, or N—R₁, Z₁ and Z₂ are independently CR₂; Y₁ and Y₂ are C; Y₃ and Y₄ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and one of Z₁ or Z₂ is a carbon to which the remainder of the molecule is attached; or

Z₃ is O, S, or N—R₁; Z₁ is N; Z₂ is CR₂; Y₁ and Y₂ are C; Y₃ and Y₄ are independently C, CH, or N (wherein a double bond can be present); W₁, W₂, W₃, W₄, and W₅ are independently N—R₁, O, S═(═O)ᵣ where r is 0-2, or CR₄R₄; t is 0-2; u is 1-3; and Z₂ is a carbon to which the remainder of the molecule is attached; or

Z₃ is O, S, or N—R₁; Z₁ is CH₂; Z₂ is N; Y₁ and Y₂ are C; Y₃ and Y₄ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and Z₁ is a carbon to which the remainder of the molecule is attached; or

Z₁, Z₂, and Z₃ are independently CR₂; Y₁ is N; Y₂ is C; Y₃ and Y₄ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and any one of Z₁, Z₂, and Z₃ is a carbon to which the remainder of the molecule is attached; or

Z₁ is N; Z₂ and Z₃ are CR₂; Y₁ is N; Y₂ is C; Y₃, and Y₄ are independently C, CH or N (wherein a double bond can be present); W₁, W₂, W₃, W₄ and W₅ are independently N—R₁, O, S(═O)ᵣ where r is 0-2, or CR₄R₄; t is 0-2; u is 1-3; and one of Z₂ or Z₃ is a carbon to which the remainder of the molecule is attached; or

Z₁ and Z₂ are N; Z₃ is CR₂; Y₁ is N; Y₂ is C; Y₃ and Y₄ are independently C, CH, or N (wherein a double bond can be present); W₁, W₂, W₃, and W₅ are independently N—R₁, O, S(═O)ᵣ where r is 0-2, or CR₄R₄; t is 0-2; u is 1-3; and Z₃ is a carbon to which the remainder of the molecule is attached; or

Z₁ and Z₃ are N; Z₂ is CR₂; Y₁ is N; Y₂ is C; Y₃ and Y₄ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and Z₂ is a carbon to which the remainder of the molecule is attached; or

Z₁, Z₂, and Z₃ are independently CR₂; Y₂ is N; Y₁ is C; Y₃ and Y₄ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and any one of Z₁, Z₂, and Z₃ is a carbon to which the remainder of the molecule is attached; or

Z₁ is N; Z₂ and Z₃ are independently CR₂; Y₂ is N; Y₁ is C; Y₃ and Y₄ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and one of Z₂ or Z₃ is a carbon to which the remainder of the molecule is attached; or

Z₁ and Z₂ are N; Z₃ is CR₂; Y₂ is N; Y₁ is C; Y₃ and Y₄ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; and Z₃ is a carbon to which the remainder of the molecule is attached; or

Z₁ and Z₃ are N; Z₂ is CR₂; Y₂ is N; Y₁ is C; Y₃ and Y₄ are independently C, CH, or N (wherein a double bond can be present); W₁, W₂, W₃, W₄, and W₅ are independently N—R₁, O, S(═O)ᵣ where r is 0-2, or CR₄R₄; t is 0-2; u is 1-3; Z₂ is a carbon to which the remainder of the molecule is attached; and

R₁, R₂, R₄, R₆ and R₇ are as defined above.

In Some Embodiments of Formula 11-A:

Z₁, Z₂, Z₃, Z₄, Z₆, Z₇, Z₈, and Z₉ are independently CR₂; Z₅ is O, S, or N—R₁; Y₁, Y₂, Y₃, and Y₄ are C; and any one of Z₁, Z₂, Z₃, Z₄, Z₆, Z₇, Z₈, and Z₉ is a carbon atom to which the remainder of the molecule is attached; or

Z₁ is N; Z₂, Z₃, Z₄, Z₆, Z₇, Z₈, and Z₉ are independently CR₂; Z₅ is O, S, or N—R₁; Y₁, Y₂, Y₃, and Y₄ are C; and any one of Z₂, Z₃, Z₄, Z₆, Z₇, Z₈, and Z₉ is a carbon atom to which the remainder of the molecule is attached; or

Z₁ and Z₂ are N; Z₃, Z₄, Z₆, Z₇, Z₈, and Z₉ are independently CR₂; Z₅ is O, S, or N—R₁; Y₁, Y₂, Y₃, and Y₄ are C; and any one of Z₃, Z₄, Z₆, Z₇, Z₈, and Z₉ is a carbon atom to which the remainder of the molecule is attached; or

Z₁ and Z₃ are N; Z₂, Z₄, Z₆, Z₇, Z₈, and Z₉ are independently CR₂; Z₅ is O, S, or N—R₁; Y₁, Y₂, Y₃, and Y₄ are C; and any one of Z₂, Z₄, Z₆, Z₇, Z₈, and Z₉ is a carbon atom to which the remainder of the molecule is attached; or

Z₁ and Z₄ are N; Z₃, Z₆, Z₇, Z₈, and Z₉ are independently CR₂; Z₅ is O, S, or N—R₁; Y₁, Y₂, Y₃, and Y₄ are C; and any one of Z₃, Z₆, Z₇, Z₈, and Z₉ is a carbon atom to which the remainder of the molecule is attached; or

Z₁ is N; Z₂, Z₃, and Z₄ are independently CR₂; Z₆, Z₇, Z₈, and Z₉ are independently N or CR₂; Z₅ is O, S, or N—R₁; Y₁, Y₂, Y₃, and Y₄ are C; and any one of Z₂, Z₃, Z₄, Z₆, Z₇, Z₈, and Z₉ is a carbon atom to which the remainder of the molecule is attached; or

Z₁ and Z₂ are N; Z₃ and Z₄ are independently CR₂; Z₆, Z₇, Z₈, and Z₉ are independently N or CR₂; Z₅ is O, S, or N—R₁; Y₁, Y₂, Y₃, and Y₄ are C; and any one of Z₃, Z₄, Z₆, Z₇, Z₈, and Z₉ is a carbon atom to which the remainder of the molecule is attached; or

Z₁ and Z₃ are N; Z₂ and Z₄ are independently CR₂; Z₆, Z₇, Z₈, and Z₉ are independently N or CR₂; Z₅ is O, S, or N—R₁; Y₁, Y₂, Y₃, and Y₄ are C; and any one of Z₂, Z₄, Z₆, Z₇, Z₈, and Z₉ is a carbon atom to which the remainder of the molecule is attached; or

Z₁ and Z₄ are N; Z₂ and Z₃ are independently CR₂; Z₆, Z₇, Z₈, and Z₉ are independently N or CR₂; Z₅ is O, S, or N—R₁; Y₁, Y₂, Y₃, and Y₄ are C; and any one of Z₂, Z₃, Z₆, Z₇, Z₈, and Z₉ is a carbon atom to which the remainder of the molecule is attached; or

Z₂ is N; Z₁, Z₃, Z₄, Z₆, Z₇, Z₈, and Z₉ are independently CR₂; Z₅ is O, S, or N—R₁; Y₁, Y₂, Y₃, and Y₄ are C; and any one of Z₁, Z₃, Z₄, Z₆, Z₇, Z₈ and Z₉ is a carbon atom to which the remainder of the molecule is attached; or

Z₂ and Z₃ are N; Z₁, Z₄, Z₆, Z₇, Z₈, and Z₉ are independently CR₂; Z₅ is O, S, or N—R₁; Y₁, Y₂, Y₃, and Y₄ are C; and any one of Z₁, Z₄, Z₆, Z₇, Z₈, and Z₉ is a carbon atom to which the remainder of the molecule is attached; or

Z₂ and Z₄ are N; Z₁, Z₃, Z₆, Z₇, Z₈, and Z₉ are independently CR₂; Z₅ is O, S, or N—R₁; Y₁, Y₂, Y₃, and Y₄ are C; and any one of Z₁, Z₃, Z₆, Z₇, Z₈, and Z₉ is a carbon atom to which the remainder of the molecule is attached; or

Z₂ is N; Z₁, Z₃, and Z₄ are independently CR₂; Z₆, Z₇, Z₈, and Z₉ are independently N or CR₂; Z₅ is O, S, or N—R₁; Y₁, Y₂, Y₃, and Y₄ are C; and any one of Z₁, Z₃, Z₄, Z₆, Z₇, Z₈, and Z₉ is a carbon atom to which the remainder of the molecule is attached; or

Z₂ and Z₃ are N; Z₁ and Z₄ are independently CR₂; Z₆, Z₇, Z₈, and Z₉ are independently N or CR₂; Z₅ is O, S, or N—R₁; Y₁, Y₂, Y₃, and Y₄ are C; and any one of Z₁, Z₄, Z₆, Z₇, Z₈, and Z₉ is a carbon atom to which the remainder of the molecule is attached; or

Z₂ and Z₄ are N; Z₁ and Z₃ are independently CR₂; Z₆, Z₇, Z₈, and Z₉ are independently N or CR₂; Z₅ is O, S, or N—R₁; Y₁, Y₂, Y₃, and Y₄ are C; any one of Z₁, Z₃, Z₆, Z₇, Z₈, and Z₉ is a carbon atom to which the remainder of the molecule is attached; and

R₁, R₂, R₄, R₆ and R₇ are as defined above.

In Some Embodiments of Formula 12-A:

Z₁, Z₂, Z₃, Z₄, Z₆, Z₇, Z₈, and Z₉ are independently CR₂; Z₅ and Z₁₀ are independently O, S, or N—R₁; Y₁, Y₂, Y₃, and Y₄ are C; and any one of Z₁, Z₂, Z₃, Z₄, Z₆, Z₇, Z₈, and Z₉ is a carbon atom to which the remainder of the molecule is attached; or

Z₁, Z₂, Z₃, Z₄, Z₆, Z₇, Z₈, and Z₉ are independently N or CR₂; Z₅ and Z₁₀ are independently O, S, or N—R₁; Y₁, Y₂, Y₃, and Y₄ are C; any one of Z₁, Z₂, Z₃, Z₄, Z₆, Z₇, Z₈, and Z₉ is a carbon atom to which the remainder of the molecule is attached; and

R₁, R₂, R₄, R₆ and R₇ are as defined above.

In Some Embodiments of Formula 12-B:

Z₁, Z₂, Z₃, Z₄, Z₇, Z₈, Z₉, and Z₁0 are independently CR₂; Z₅ and Z₆ are independently N or CR₂; Y₁, Y₂, Y₃, and Y₄ are C; and any one of Z₁, Z₂, Z₃, Z₄, Z₇, Z₈, Z₉, and Z₁₀ is a carbon atom to which the remainder of the molecule is attached; or

Z₁, Z₂, Z₃, and Z₄ are independently CR₂ or N; Z₅, Z₆, Z₇, Z₈, Z₉, and Z₁₀ are independently CR₂; Y₁, Y₂, Y₃, and Y₄ are C; any one of Z₁, Z₂, Z₃, Z₄, Z₇, Z₈, Z₉, and Z₁₀ is a carbon atom to which the remainder of the molecule is attached; and

R₁, R₂, R₄, R₆ and R₇ are as defined above.

In Some Embodiments of Formula 12-C:

Z₁, Z₂, Z₃, Z₄, Z₆, Z₇, Z₈, and Z₉ are independently CR₂; Y₁ is N; Y₂ is C; and any one of Z₁, Z₂, Z₃, Z₄, Z₆, Z₇, Z₈, and Z₉ is a carbon atom to which the remainder of the molecule is attached; or

Z₁, Z₂, Z₃, Z₄, Z₇, Z₈, and Z₉ are independently CR₂; Z₆ is O, S, or N—R₁; Y₁ is C; Y₂ is N; any one of Z₁, Z₂, Z₃, Z₄, Z₆, Z₇, Z₈, and Z₉ is a carbon atom to which the remainder of the molecule is attached; and

R₁, R₂, R₄, R₆ and R₇ are as defined above.

In Some Embodiments of Formula 13-A:

Z₁, Z₂, Z₃, and Z₄ are independently CR₂; Z₅ is O, S, or N—R₁; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, Y₃, and Y₄ are C; and any one of Z₁, Z₂, Z₃, and Z₄ is a carbon atom to which the remainder of the molecule is attached; or

Z₁, Z₂, Z₃, and Z₄ are independently CR₂; Z₅ is N or CR₂; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, and Y₃ are C; Y₄ is N; and any one of Z₁, Z₂, Z₃, and Z₄ is a carbon atom to which the remainder of the molecule is attached; or

Z₁, Z₂, Z₃, and Z₄ are independently CR₂; Z₅ is O, S, or N; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-3; Y₁, Y₂, and Y₃ are C; Y₄ is N; and any one of Z₁, Z₂, Z₃, and Z₄ is a carbon atom to which the remainder of the molecule is attached; or

Z₁ is N; Z₂, Z₃, and Z₄ are independently CR₂; Z₅ is O, S, or N—R₁; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, Y₃, and Y₄ are C; and any one of Z₂, Z₃, and Z₄ is a carbon atom to which the remainder of the molecule is attached; or

Z₁ and Z₂ are N; Z₃ and Z₄ are independently CR₂; Z₅ is O, S, or N—R₁; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₂, Y₃, and Y₄ are C; and one of Z₃ or Z₄ is a carbon atom to which the remainder of the molecule is attached; or

Z₁ and Z₃ are N; Z₂ and Z₄ are independently CR₂; Z₅ is O, S, or N—R₁; Z₁ and Z₂ are N; Z₃ and Z₄ are independently CR₂; Z₅ is N or CR₂; W₁, W₂, and W₃ are independently CR₄R₄, S(═O)r where r is 0-2, O, or N—R₁; with the proviso that whenever W₂ is S or O it is not adjacent to a W1, W2 or W3 that is also S or O with the proviso that whenever W2 is S or O it is not adjacent to a W1, W2 or W3 that is also S or O and wherein one double bond may be present; t is 1-4; Y₁, Y₂, and Y₃ are C; Y₄ is N; and one of Z₂ or Z₄ is a carbon atom to which the remainder of the molecule is attached; or

Z₁ and Z₄ are N; Z₂ and Z₃ are independently CR₂; Z₅ is O, S, or N—R₁; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, Y₃, and Y₄ are C; and one of Z₂ or Z₃ is a carbon atom to which the remainder of the molecule is attached; or

Z₁ is N; Z₂, Z₃, and Z₄ are independently CR₂; Z₅ is N or CR₂; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, and Y₃ are C; Y₄ is N; and any one of Z₂, Z₃, and Z₄ is a carbon atom to which the remainder of the molecule is attached; or

Z₁ and Z₃ are N; Z₂ and Z₄ are independently CR₂; Z₅ is N or CR₂; W₁, W₂, and W₃ are independently CR₄R₄, S(═O)r where r is 0-2, O, or N—R₁; with the proviso that whenever W2 is S or O it is not adjacent to a W1, W2 or W3 that is also S or O and wherein one double bond may be present; t is 1-4; Y₁, Y₂, and Y₃ are C; Y₄ is N; and one of Z₂ or Z₄ is a carbon atom to which the remainder of the molecule is attached; or

Z₁ and Z₄ are N; Z₂ and Z₃ are independently CR₂; Z₅ is N or CR₂; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, and Y₃ are C; Y₄ is N; and one of Z₂ and Z₃ is a carbon atom to which the remainder of the molecule is attached; or

Z₁ and Z₂ are N; Z₃ and Z₄ are independently CR₂; Z₅ is O, S or N; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-3; Y₁, Y₂, and Y₃ are C; Y₄ is N; and one of Z₃ or Z₄ is a carbon atom to which the remainder of the molecule is attached; or

Z₁ and Z₃ are N; Z₂ and Z₄ are independently CR₂; Z₅ is O, S, or N; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-3; Y₁, Y₂, and Y₃ are C; Y₄ is N; and one of Z₂ and Z₄ is a carbon atom to which the remainder of the molecule is attached; or

Z₁ and Z₄ are N; Z₂ and Z₃ are independently CR₂; Z₅ is O, S, or N; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-3; Y₁, Y₂, Y₃ are C; Y₄ is N; and one of Z₂ or Z₃ is a carbon atom to which the remainder of the molecule is attached; or

Z₂ is N; Z₁, Z₃, and Z₄ are independently CR₂; Z₅ is O, S, or N—R₁; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and one double bond may be present; t is 1-4; Y₁, Y₂, Y₃, and Y₄ are C; and any one of Z₁, Z₃, and Z₄ is a carbon atom to which the remainder of the molecule is attached; or

Z₂ and Z₃ are N; Z₁ and Z₄ are independently CR₂; Z₅ is O, S, or N—R₁; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, Y₃, and Y₄ are C; and one of Z₁ or Z₄ is a carbon atom to which the remainder of the molecule is attached; or

Z₂ and Z₄ are N; Z₁ and Z₃ are independently CR₂; Z₅ is O, S, or N—R₁; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, Y₃, and Y₄ are C; and one of Z₁ or Z₃ is a carbon atom to which the remainder of the molecule is attached; or

Z₂ is N; Z₁, Z₃, and Z₄ are independently CR₂; Z₅ is N or CR₂; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, and Y₃ are C; Y₄ is N; and any one of Z₁, Z₃, and Z₄ is a carbon atom to which the remainder of the molecule is attached; or

Z₂ and Z₃ are N; Z₁ and Z₄ are independently CR₂; Z₅ is N or CR₂; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, and Y₃ are C; Y₄ is N; and one of Z₁ and Z₄ is a carbon atom to which the remainder of the molecule is attached; or

Z₂ and Z₄ are N; Z₁ and Z₃ are independently CR₂; Z₅ is N or CR₂; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, Y₃ are C; Y₄ is N; and one of Z₁ or Z₃ is a carbon atom to which the remainder of the molecule is attached; or

Z₂ is N; Z₁, Z₃, and Z₄ are independently CR₂; Z₅ is O, S, or N; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-3; Y₁, Y₂, Y₃ are C; Y₄ is N; and any one of Z₁, Z₃, and Z₄ is a carbon atom to which the remainder of the molecule is attached; or

Z₂ and Z₃ are N; Z₁ and Z₄ are independently CR₂; Z₅ is O, S, or N; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-3; Y₁, Y₂, Y₃ are C; Y₄ is N; and one of Z₁ or Z₄ is a carbon atom to which the remainder of the molecule is attached; or

Z₂ and Z₄ are N; Z₁ and Z₃ are independently CR₂, Z₅ is O, S, or N; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-3; Y₁, Y₂, Y₃ are C; Y₄ is N; and one of Z₁ or Z₃ is a carbon atom to which the remainder of the molecule is attached; or

Z₃ is N; Z₁, Z₂, and Z₄ are independently CR₂; Z₅ is O, S, or N—R₁; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, Y₃, and Y₄ are C; and any one of Z₁, Z₂, and Z₄ is a carbon atom to which the remainder of the molecule is attached; or

Z₃ and Z₄ are N; Z₁ and Z₄ are independently CR₂; Z₅ is O, S, or N—R₁; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, Y₃, and Y₄ are C; and one of Z₁ or Z₂ is a carbon atom to which the remainder of the molecule is attached; or

Z₃ is N; Z₁, Z₃, and Z₄ are independently CR₂; Z₅ is N or CR₂; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, and Y₃ are C; Y₄ is N; and any one of Z₁, Z₂, and Z₄ is a carbon atom to which the remainder of the molecule is attached; or

Z₃ and Z₄ are N; Z₁ and Z₂ are independently CR₂; Z₅ is N or CR₂; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, and Y₃ are C; Y₄ is N; and one of Z₁ or Z₂ is a carbon atom to which the remainder of the molecule is attached; or

Z₃ is N; Z₁, Z₃, and Z₄ are independently CR₂; Z₅ is O, S, or N; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-3; Y₁, Y₂, and Y₃ are C; Y₄ is N; and any one of Z₁, Z₂, and Z₄ is a carbon atom to which the remainder of the molecule is attached; or

Z₃ and Z₄ are N; Z₁ and Z₄ are independently CR₂; Z₅ is O, S, or N; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-3; Y₂, and Y₃ are C; Y₄ is N; and one of Z₁ or Z₂ is a carbon atom to which the remainder of the molecule is attached; or

Z₁, Z₂, and Z₃ are independently CR₂; Z₄ is N; Z₅ is O, S, or N—R₁; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, Y₃, and Y₄ are C; and any one of Z₁, Z₂, and Z₃, is a carbon atom to which the remainder of the molecule is attached; or

Z₁, Z₂, and Z₃ are independently CR₂; Z₄ is N; Z₅ is N or CR₂; W₁, W₂, and W₃ are independently CR₄R₄, S(═O)r where r is 0-2, O, or N—R₁ with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-4; Y₁, Y₂, and Y₃ are C; Y₄ is N; and any one of Z₁, Z₂, and Z₃ is a carbon atom to which the remainder of the molecule is attached; or

Z₁, Z₂, and Z₃ are independently CR₂; Z₄ is N; Z₅ is O, S, or N; W₁, W₂, and W₃ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O and wherein one double bond may be present; t is 1-3; Y₁, Y₂, and Y₃ are C; Y₄ is N; any one of Z₁, Z₂, Z₃, and Z₄ is a carbon atom to which the remainder of the molecule is attached; and

R₁, R₂, R₄, R₆ and R₇ are as defined above.

In Some Embodiments of Formula 13-B:

Z₁, Z₂, Z₃, and Z₄ are independently CR₂; Z₅ is O, S, or N—R₁; Y₁, Y₂, Y₃, and Y₄ are C; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and any one of Z₁, Z₂, Z₃, and Z₄ is a carbon atom to which the remainder of the molecule is attached; or

Z₁ is N; Z₂, Z₃, and Z₄ are independently CR₂; Z₅ is O, S, or N—R₁; Y₁, Y₂, Y₃, and Y₄ are C; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and any one of Z₂, Z₃, and Z₄ is a carbon atom to which the remainder of the molecule is attached; or

Z₁ and Z₂ are N; Z₃ and Z₄ are independently CR₂; Z₅ is O, S, or N—R₁; Y₁, Y₂, Y₃, and Y₄ are C; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and one of Z₃ or Z₄ is a carbon atom to which the remainder of the molecule is attached; or

Z₁ and Z₃ are N; Z₂ and Z₄ are independently CR₂; Z₅ is O, S, or N—R₁; Y₁, Y₂, Y₃, and Y₄ are C; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and one of Z₂ or Z₄ is a carbon atom to which the remainder of the molecule is attached; or

Z₂ is N; Z₁, Z₃, and Z₄ are independently CR₂; Z₅ is O, S, or N—R₁; Y₁, Y₂, Y₃, and Y₄ are C; W₁ and W2 are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and any one of Z₁, Z₃, and Z₄ is a carbon atom to which the remainder of the molecule is attached; or

Z₂ and Z₃ are N; Z₁ and Z₄ are independently CR₂; Z₅ is O, S, or N—R₁; Y₁, Y₂, Y₃, and Y₄ are C; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and one of Z₁ or Z₄ is a carbon atom to which the remainder of the molecule is attached; or

Z₃ is N; Z₁, Z₂, and Z₄ are independently CR₂; Z₅ is O, S, or N—R₁; Y₁, Y₂, Y₃, and Y₄ are C; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and any one of Z₁, Z₂, and Z₄ is a carbon atom to which the remainder of the molecule is attached; or

Z₁, Z₂, Z₃, and Z₅ are independently CR₂; Z₄ is O, S, or N—R₁; Y₁, Y₂, Y₃, and Y₄ are C; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and any one of Z₁, Z₂, Z₃, and Z₅ is a carbon atom to which the remainder of the molecule is attached; or

Z₁ is N; Z₂, Z₃, and Z₅ are independently CR₂; Z₄ is O, S, or N—R₁; Y₁, Y₂, Y₃, and Y₄ are C; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and any one of Z₂, Z₃, and Z₅ is a carbon atom to which the remainder of the molecule is attached; or

Z₁ and Z₂ are N; Z₃ and Z₅ are independently CR₂; Z₄ is O, S, or N—R₁; Y₁, Y₂, Y₃, and Y₄ are C; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and one of Z₃ or Z₅ is a carbon atom to which the remainder of the molecule is attached; or

Z₁ and Z₃ are N; Z₂ and Z₅ are independently CR₂; Z₄ is O, S, or N—R₁; Y₁, Y₂, Y₃, and Y₄ are C; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and one of Z₂ or Z₅ is a carbon atom to which the remainder of the molecule is attached; or

Z₂ is N; Z₁, Z₃, and Z₅ are independently CR₂; Z₄ is O, S, or N—R₁; Y₁, Y₂, Y₃, and Y₄ are C; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and any one of Z₁, Z₃, and Z₅ is a carbon atom to which the remainder of the molecule is attached; or

Z₂ and Z₃ are N; Z₁ and Z₅ are independently CR₂; Z₄ is O, S or N—R₁; Y₁, Y₂, Y₃, and Y₄ are C; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and one of Z₁ or Z₅ is a carbon atom to which the remainder of the molecule is attached; or

Z₃ is N; Z₁, Z₂, and Z₅ are independently CR₂; Z₄ is O, S, or N—R₁; Y₁, Y₂, Y₃, and Y₄ are C; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and any one of Z₁, Z₂, and Z₅ is a carbon atom to which the remainder of the molecule is attached; or

Z₁, Z₂, Z₃, Z₄, and Z₅ are independently CR₂; Y₁, Y₂, and Y₃ are C; Y₄ is N; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and any one of Z₁, Z₂, Z₃, and Z₄ is a carbon atom to which the remainder of the molecule is attached; or

Z₁ is N; Z₂, Z₃, Z₄, and Z₅ are independently CR₂; Y₁, Y₂, and Y₃ are C; Y₄ is N; W₁ and W₂ are independently CR₄R₄, S(═O)r where r is 0-2, O, or N—R₁; with the proviso that whenever W₂ is S or O it is not adjacent to a W₁, W₂ or W₃ that is also S or O with the proviso that whenever W2 is S or O it is not adjacent to a W1, W2 or W3 that is also S or O; t is 1-3; and any one of Z₂, Z₃, and Z₄ is a carbon atom to which the remainder of the molecule is attached; or

Z₁ and Z₂ are N; Z₃, Z₄, and Z₅ are independently CR₂; Y₁, Y₂, and Y₃ are C; Y₄ is N; W₁ and W₂ are independently CR₄R₄, S(═O)r where r is 0-2, O, or N—R₁; with the proviso that whenever W2 is S or O it is not adjacent to a W1, W2 or W3 that is also S or O; t is 1-3; and one of Z₃ or Z₄ is a carbon atom to which the remainder of the molecule is attached; or

Z₁ and Z₃ are N; Z₂, Z₄, and Z₅ are independently CR₂; Y₁, Y₂, and Y₃ are C; Y₄ is N; W₁ and W₂ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₄, and Z₅ is a carbon atom to which the remainder of the molecule is attached; or

Z₂ is N; Z₁, Z₃, Z₄, and Z₅ are independently CR₂; Y₁, Y₂, and Y₃ are C; Y₄ is N; W₁ and W₂ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, and Z₄ is a carbon atom to which the remainder of the molecule is attached; or

Z₂ and Z₃ are N; Z₁, Z₄, and Z₅ are independently CR₂; Y₁, Y₂, and Y₃ are C; Y₄ is N; W₁ and W₂ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₄, and Z₅ is a carbon atom to which the remainder of the molecule is attached; or

Z₃ is N; Z₁, Z₂, Z₄, and Z₅ are independently CR₂; Y₁, Y₂, and Y₃ are C; Y₄ is N; W₁ and W₂ are independently CR₄R₄, S(═O)r where r is 0-2, O, or N—R₁; with the proviso that whenever W2 is S or O it is not adjacent to a W1, W2 or W3 that is also S or O; t is 1-3; any one of Z₁, Z₂, Z₄, and Z₅ is a carbon atom to which the remainder of the molecule is attached; and

R₁, R₂, R₄, R₆ and R₇ are as defined above.

In Some Embodiments of Formula 14-A:

Z₁, Z₂, Z₃, Z₄, Z₅, and Z₆ are independently N or CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; any one of Z₁, Z₂, Z₃, Z₄, Z₅, and Z₆ is a carbon atom to which the remainder of the molecule is attached; and

R₁, R₂, R₄, R₆ and R₇ are as defined above.

Z₁ is N; Z₂, Z₃, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; any one of Z₂, Z₃, Z₄, Z₅, and Z₆ is a carbon atom to which the remainder of the molecule is attached; or

Z₁ and Z₂ are N; Z₃, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₃, Z₄, Z₅, and Z₆ is a carbon atom to which the remainder of the molecule is attached; or

Z₁ and Z₃ are N; Z₂, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₄, Z₅, and Z₆ is a carbon atom to which the remainder of the molecule is attached; or

Z₁ and Z₄ are N; Z₂, Z₃, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₃, Z₅, and Z₆ is a carbon atom to which the remainder of the molecule is attached; or

Z₁ and Z₅ are N; Z₂, Z₃, Z₄, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₃, Z₄, and Z₆ is a carbon atom to which the remainder of the molecule is attached; or

Z₁ and Z₆ are N; Z₂, Z₃, Z₄, and Z₅ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₃, Z₄, and Z₅ is a carbon atom to which the remainder of the molecule is attached; or

Z₂ is N Z₁, Z₃, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄, Z₅, and Z₆ is a carbon atom to which the remainder of the molecule is attached; or

Z₂ and Z₃ are N; Z₁, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₄, Z₅, and Z₆ is a carbon atom to which the remainder of the molecule is attached; or

Z₂ and Z₄ are N; Z₁, Z₃, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₅, and Z₆ is a carbon atom to which the remainder of the molecule is attached; or

Z₂ and Z₅ are N; Z₁, Z₃, Z₄, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄, and Z₆ is a carbon atom to which the remainder of the molecule is attached; or

Z₂ and Z₅ are N; Z₁, Z₃, Z₄, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄, and Z₆ is a carbon atom to which the remainder of the molecule is attached; or

Z₂ and Z₆ are N; Z₁, Z₃, Z₄, and Z₅ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄, and Z₅ is a carbon atom to which the remainder of the molecule is attached; or

Z₃ is N; Z₁, Z₂, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₄, Z₅, and Z₆ is a carbon atom to which the remainder of the molecule is attached; or

Z₃ and Z₄ are N; Z₁, Z₂, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₅, and Z₆ is a carbon atom to which the remainder of the molecule is attached; or

Z₃ and Z₅ are N; Z₁, Z₂, Z₄, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₄, and Z₆ is a carbon atom to which the remainder of the molecule is attached; or

Z₃ and Z₆ are N; Z₁ Z₂, Z₄, and Z₅ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₄, and Z₅ is a carbon atom to which the remainder of the molecule is attached; or

Z₄ is N; Z₁, Z₂, Z₃, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃ Z₅, and Z₆ is a carbon atom to which the remainder of the molecule is attached; or

Z₄ and Z₅ are N; Z₁, Z₂, Z₃, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, Z₅, and Z₆ is a carbon atom to which the remainder of the molecule is attached; or

Z₅ is N; Z₁, Z₂, Z₃, Z₄, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, Z₄, and Z₆ is a carbon atom to which the remainder of the molecule is attached; or

Z₅ and Z₆ are N; Z₁, Z₂, Z₃, and Z₄ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, and Z₄ is a carbon atom to which the remainder of the molecule is attached; or

Z₆ is N; Z₁, Z₂, Z₃, Z₄, and Z₅ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, Z₄, and Z₅ is a carbon atom to which the remainder of the molecule is attached.

In Some Embodiments of Formula 14-B:

Z₁, Z₂, Z₃, Z₄, Z₅, and Z₆ are independently N or CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, Z₄, Z₅, and Z₆ is a carbon atom to which the remainder of the molecule is attached; or

Z₁ is N; Z₂, Z₃, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₃, Z₄, Z₅, and Z₆ is a carbon atom to which the remainder of the molecule is attached; or

Z₁ and Z₂ are N; Z₃, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₃, Z₄, Z₅, and Z₆ is a carbon atom to which the remainder of the molecule is attached; or

Z₁ and Z₃ are N; Z₂, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₄, Z₅, and Z₆ is a carbon atom to which the remainder of the molecule is attached; or

Z₁ and Z₄ are N; Z₂, Z₃, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₃, Z₅, and Z₆ is a carbon atom to which the remainder of the molecule is attached; or

Z₁ and Z₅ are N; Z₂, Z₃, Z₄, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₃, Z₄, and Z₆ is a carbon atom to which the remainder of the molecule is attached; or

Z₁ and Z₆ are N; Z₂, Z₃, Z₄, and Z₅ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₃, Z₄, and Z₅ is a carbon atom to which the remainder of the molecule is attached; or

Z₂ is N; Z₁, Z₃, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄, Z₅, and Z₆ is a carbon atom to which the remainder of the molecule is attached; or

Z₂ and Z₃ are N; Z₁, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₄, Z₅, and Z₆ is a carbon atom to which the remainder of the molecule is attached; or

Z₂ and Z₄ are N; Z₁, Z₃, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₅, and Z₆ is a carbon atom to which the remainder of the molecule is attached; or

Z₂ and Z₅ are N; Z₁, Z₃, Z₄, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄, and Z₆ is a carbon atom to which the remainder of the molecule is attached; or

Z₂ and Z₅ are N; Z₁, Z₃, Z₄, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄, and Z₆ is a carbon atom to which the remainder of the molecule is attached; or

Z₂ and Z₆ are N; Z₁, Z₃, Z₄, and Z₅ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄, and Z₅ is a carbon atom to which the remainder of the molecule is attached; or

Z₃ is N; Z₁, Z₂, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₄, Z₅, and Z₆ is a carbon atom to which the remainder of the molecule is attached; or

Z₃ and Z₄ are N; Z₁, Z₂, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₅, and Z₆ is a carbon atom to which the remainder of the molecule is attached; or

Z₃ and Z₅ are N; Z₁, Z₂, Z₄, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₄, and Z₆ is a carbon atom to which the remainder of the molecule is attached; or

Z₃ and Z₆ are N; Z₁, Z₂, Z₄, and Z₅ are independently CR₂; Y₁, Y₂, Y₃ and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₄, and Z₅ is a carbon atom to which the remainder of the molecule is attached; or

Z₄ is N; Z₁, Z₂, Z₃, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, Z₅, and Z₆ is a carbon atom to which the remainder of the molecule is attached; or

Z₄ and Z₅ are N; Z₁, Z₂, Z₃, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, Z₅, and Z₆ is a carbon atom to which the remainder of the molecule is attached; or

Z₅ is N; Z₁, Z₂, Z₃, Z₄, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, Z₄, and Z₆ is a carbon atom to which the remainder of the molecule is attached; or

Z₅ and Z₆ are N; Z₁, Z₂, Z₃, and Z₄ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, and Z₄ is a carbon atom to which the remainder of the molecule is attached; or

Z₆ is N; Z₁, Z₂, Z₃, Z₄, and Z₅ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, Z₄, and Z₅ is a carbon atom to which the remainder of the molecule is attached.

In Some Embodiments of Formula 14-C:

Z₁, Z₂, Z₃, Z₄, Z₅, and Z₆ are independently N or CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, Z₄, Z₅, and Z₆ is a carbon atom to which the remainder of the molecule is attached; or

Z₁ is N; Z₂, Z₃, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁ with the proviso that whenever W2 is S or O it is not adjacent to a W1, W2 or W3 that is also S or O; t is 1-3; and any one of Z₂, Z₃, Z₄, Z₅, and Z₆ is a carbon atom to which the remainder of the molecule is attached; or

Z₁ and Z₂ are N; Z₃, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁ with the proviso that whenever W2 is S or O it is not adjacent to a W1, W2 or W3 that is also S or O; t is 1-3; and any one of Z₃, Z₄, Z₅, and Z₆ is a carbon atom to which the remainder of the molecule is attached; or

Z₁ and Z₃ are N; Z₂, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₄, Z₅, and Z₆ is a carbon atom to which the remainder of the molecule is attached; or

Z₁ and Z₄ are N; Z₂, Z₃, Z₅, and Z₆ are independently CR₂; Y₁, Y₂, Y₃, and Y₃ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₃, Z₅, and Z₆ is a carbon atom to which the remainder of the molecule is attached; or

Z₁ and Z₅ are N; Z₂, Z₃, Z₄, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₃, Z₄, and Z₆ is a carbon atom to which the remainder of the molecule is attached; or

Z₁ and Z₆ are N; Z₂, Z₃, Z₄, and Z₅ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₃, Z₄, and Z₅ is a carbon atom to which the remainder of the molecule is attached; or

Z₂ is N; Z₁, Z₃, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄ Z₅, and Z₆ is a carbon atom to which the remainder of the molecule is attached; or

Z₂ and Z₃ are N; Z₁, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₄, Z₅, and Z₆ is a carbon atom to which the remainder of the molecule is attached; or

Z₂ and Z₄ are N; Z₁, Z₃, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₅, and Z₆ is a carbon atom to which the remainder of the molecule is attached; or

Z₂ and Z₅ are N; Z₁, Z₃, Z₄, and Z₆ are independently CR₂; Y₁, Y₂, Y₃, Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄, and Z₆ is a carbon atom to which the remainder of the molecule is attached; or

Z₂ and Z₅ are N; Z₁, Z₃, Z₄, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄, and Z₆ is a carbon atom to which the remainder of the molecule is attached; or

Z₂ and Z₆ are N; Z₁, Z₃, Z₄, and Z₅ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄, and Z₅ is a carbon atom to which the remainder of the molecule is attached; or

Z₃ is N; Z₁, Z₂, Z₄, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₄, Z₅, and Z₆ is a carbon atom to which the remainder of the molecule is attached; or

Z₃ and Z₄ are N; Z₁, Z₂, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₅, and Z₆ is a carbon atom to which the remainder of the molecule is attached; or

Z₃ and Z₅ are N; Z₁, Z₂, Z₄, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₄, and Z₆ is a carbon atom to which the remainder of the molecule is attached; or

Z₃ and Z₆ are N; Z₁, Z₂, Z₄, and Z₅ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₄, and Z₅ is a carbon atom to which the remainder of the molecule is attached; or

Z₄ is N; Z₁, Z₂, Z₃, Z₅, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, Z₅, and Z₆ is a carbon atom to which the remainder of the molecule is attached; or

Z₄ and Z₅ are N; Z₁, Z₂, Z₃, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, Z₅, and Z₆ is a carbon atom to which the remainder of the molecule is attached; or

Z₅ is N; Z₁, Z₂, Z₃, Z₄, and Z₆ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, Z₄, and Z₆ is a carbon atom to which the remainder of the molecule is attached; or

Z₅ and Z₆ are N; Z₁, Z₂, Z₃, and Z₄ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, and Z₄ is a carbon atom to which the remainder of the molecule is attached; or

Z₆ is N; Z₁, Z₂, Z₃, Z₄, and Z₅ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₂, Z₃, Z₄, and Z₅ is a carbon atom to which the remainder of the molecule is attached; or

In Some Embodiments of Formula 15-A and 15-B:

Z₁ is N; Z₂, Z₃, Z₄, Z₅, Z₆, Z₇, and Z₈ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₃, Z₄, Z₅, Z₆, Z₇, and Z₈ is a carbon atom to which the remainder of the molecule is attached; or

Z₁ and Z₂ are N; Z₃, Z₄, Z₅, Z₆, Z₇, and Z₈ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₃, Z₄, Z₅, Z₆, Z₇, and Z₈ is a carbon atom to which the remainder of the molecule is attached; or

Z₁ and Z₃ are N; Z₂, Z₄, Z₅, Z₆, Z₇, and Z₈ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₄, Z₅, Z₆, Z₇, and Z₈ is a carbon atom to which the remainder of the molecule is attached; or

Z₁ and Z₄ are N; Z₂, Z₃, Z₅, Z₆, Z₇, and Z₈ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₃, Z₅, Z₆, Z₇, and Z₈ is a carbon atom to which the remainder of the molecule is attached; or

Z₁ and Z₂ are N; Z₃, Z₄, Z₅, Z₆, Z₇, and Z₈ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₃, Z₄, Z₅, Z₆, Z₇, and Z₈ is a carbon atom to which the remainder of the molecule is attached; or

Z₁ is N; Z₂, Z₃, and Z₄ are independently CR₂; Z₅, Z₆, Z₇, and Z₈ are independently N or CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₃, Z₄, Z₅, Z₆, Z₇, and Z₈ is a carbon atom to which the remainder of the molecule is attached; or

Z₁ and Z₂ are N; Z₃ and Z₄ are independently CR₂; Z₅, Z₆, Z₇, and Z₈ are independently N or CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₃, Z₄, Z₅, Z₆, Z₇, and Z₈ is a carbon atom to which the remainder of the molecule is attached; or

Z₁ and Z₃ are N; Z₂ and Z₄ are independently CR₂; Z₅, Z₆, Z₇, and Z₈ are independently N or CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₄, Z₅, Z₆, Z₇, and Z₈ is a carbon atom to which the remainder of the molecule is attached; or

Z₁ and Z₄ are N; Z₂ and Z₃ are independently CR₂; Z₅, Z₆, Z₇, and Z₈ are independently N or CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₂, Z₃, Z₅, Z₆, Z₇, and Z₈ is a carbon atom to which the remainder of the molecule is attached; or

Z₁ and Z₂ are N; Z₃ and Z₄ are independently CR₂; Z₅, Z₆, Z₇, and Z₈ are independently N or CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₃, Z₄, Z₅, Z₆, Z₇, and Z₈ is a carbon atom to which the remainder of the molecule is attached; or

Z₂ is N; Z₁, Z₃, Z₄, Z₅, Z₆, Z₇, and Z₈ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄, Z₅, Z₆, Z₇, and Z₈ is a carbon atom to which the remainder of the molecule is attached; or

Z₂ and Z₃ are N; Z₁, Z₄, Z₅, Z₆, Z₇, and Z₈ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₄, Z₅, Z₆, Z₇, and Z₈ is a carbon atom to which the remainder of the molecule is attached; or

Z₂ and Z₄ are N; Z₁, Z₃, Z₅, Z₆, Z₇, and Z₈ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₅, Z₆, Z₇, and Z₈ is a carbon atom to which the remainder of the molecule is attached; or

Z₂ is N; Z₁, Z₃, and Z₄ are independently CR₂; Z₅, Z₆, Z₇, and Z₈ are independently N or CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄, Z₅, Z₆, Z₇, and Z₈ is a carbon atom to which the remainder of the molecule is attached; or

Z₂ and Z₃ are N; Z₁ and Z₄ are independently CR₂; Z₅, Z₆, Z₇, and Z₈ are independently N or CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₄, Z₅, Z₆, Z₇, and Z₈ is a carbon atom to which the remainder of the molecule is attached; or

Z₂ and Z₄ are N; Z₁ and Z₃ are independently CR₂; Z₅, Z₆, Z₇, and Z₈ are independently N or CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₅, Z₆, Z₇, and Z₈ is a carbon atom to which the remainder of the molecule is attached; or

Z₃ is N; Z₁, Z₂, Z₄, Z₅, Z₆, Z₇, and Z₈ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or ; t is 1-3; and any one of Z₁, Z₂, Z₄, Z₅, Z₆, Z₇, and Z₈ is a carbon atom to which the remainder of the molecule is attached; or

Z_{3 l and Z}₄ are N; Z₁, Z₂, Z₅, Z₆, Z₇, and Z₈ are independently CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or ; t is 1-3; and any one of Z₁, Z₂, Z₅, Z₆, Z₇, and Z₈ is a carbon atom to which the remainder of the molecule is attached; or

Z₄ is N; Z₁, Z₃, and Z₄ are independently CR₂; Z₅, Z₆, Z₇, and Z₈ are independently N or CR₂; Y₁, Y₂, Y₃, and Y₄ are C; W₁, W₂, and W₃ are independently CR₄R₄, S(═O)r where r is 0-2, O, or N—R₁; with the proviso that whenever W2 is S or O it is not adjacent to a W1, W2 or W3 that is also S or O; t is 1-3; and any one of Z₁, Z₃, Z₄, Z₅, Z₆, Z₇, and Z₈ is a carbon atom to which the remainder of the molecule is attached.

In Some Embodiments of Formula 16-A:

Z₁ is N; Z₂, Z₃, and Z₄ are independently CR₂; Y₁ and Y₂ are C; Y₃ and Y₄ are independently CH or N; W₁ is O, S(═O)r where r is 0-2, CR₄R₄, N—R₁, or a bond; W₂ is O, S(═O)r where r is 0-2, CR₄R₄, or N—R₁; t is 0-2; W₃, W₄, and W₅ are independently O, S(═O)r where r is 0-2, CR₄R₄, or N—R₁; with the proviso that whenever W2 is S or O it is not adjacent to a W1, W2 or W3 that is also S or O; u is 1-3; and any one of Z₂, Z₃, and Z₄ is a carbon atom to which the remainder of the molecule is attached; or

Z₁ and Z₂ are N; Z₃ and Z₄ are independently CR₂; Y₁ and Y₂ are C; Y₃ and Y₄ are independently CH or N; W₁ is O, S(═O)r where r is 0-2, CR₄R₄, N—R₁, or a bond; W₂ is O, S(═O)r where r is 0-2, CR₄R₄, or N—R₁; t is 0-2; W₃, W₄, and W₅ are independently O, S(═O)r where r is 0-2, CR₄R₄, or N—R₁; with the proviso that whenever W2 is S or O it is not adjacent to a W1, W2 or W3 that is also S or O; u is 1-3; and one of Z₃ or Z₄ is a carbon atom to which the remainder of the molecule is attached; or

Z₁ and Z₃ are N; Z₂ and Z₄ are independently CR₂; Y₁ and Y₂ are C; Y₃ and Y₄ are independently CH or N; W₁ is O, S(═O)r where r is 0-2, CR₄R₄, N—R₁, or a bond; W₂ is 0, S(═O)r where r is 0-2, CR₄R₄, or N—R₁; t is 0-2; W₃, W₄, and W₅ are independently O, S(═O)r where r is 0-2, CR₄R₄, or N—R₁; with the proviso that whenever W2 is S or O it is not adjacent to a W1, W2 or W3 that is also S or O; u is 1-3; and one of Z₂ or Z₄ is a carbon atom to which the remainder of the molecule is attached; or

Z₁ and Z₄ are N; Z₂ and Z₃ are independently CR₂; Y₁ and Y₂ are C; Y₃ and Y₄ are independently CH or N; W₁ is O, S(═O)r where r is 0-2, CR₄R₄, N—R₁, or a bond; W₂ is O, S(═O)r where r is 0-2, CR₄R₄, or N—R₁; t is 0-2; W₃, W₄, and W₅ are independently O, S(═O)r where r is 0-2, CR₄R₄, or N—R₁; with the proviso that whenever W2 is S or O it is not adjacent to a W1, W2 or W3 that is also S or O; u is 1-3; and one of Z₂ or Z₃ is a carbon atom to which the remainder of the molecule is attached; or

Z₂ is N; Z₁, Z₃, and Z₄ are independently CR₂; Y₁ and Y₂ are C; Y₃ and Y₄ are independently CH or N; W₁ is O, S(═O)r where r is 0-2, CR₄R₄, N—R₁, or a bond; W₂ is O, S(═O)r where r is 0-2, CR₄R₄, or N—R₁; t is 0-2; W₃, W₄, W₄, and W₅ are independently O, S(═O)r where r is 0-2, CR₄R₄, or N—R₁; with the proviso that whenever W2 is S or O it is not adjacent to a W1, W2 or W3 that is also S or O; u is 1-3; and any one of Z₁, Z₃, and Z₄ is a carbon atom to which the remainder of the molecule is attached; or

Z₂ and Z₃ are N; Z₁ and Z₄ are independently CR₂; Y₁ and Y₂ are C; Y₃ and Y₄ are independently CH or N; W₁ is O, S(═O)r where r is 0-2, CR₄R₄, N—R₁, or a bond; W₂ is O, S(═O)r where r is 0-2, CR₄R₄, or N—R₁; t is 0-2; W₃, W₄, and W₅ are independently O, S(═O)r where r is 0-2, CR₄R₄, or N—R₁; with the proviso that whenever W2 is S or O it is not adjacent to a W1, W2 or W3 that is also S or O; u is 1-3; and one of Z₁ or Z₄ is a carbon atom to which the remainder of the molecule is attached; or

Z₂ and Z₄ are N; Z₁ and Z₃ are independently CR₂; Y₁ and Y₂ are C; Y₃ and Y₄ are independently CH or N; W₁ is O, S(═O)r where r is 0-2, CR₄R₄, N—R₁, or a bond; W₂ is O, S(═O)r where r is 0-2, CR₄R₄, or N—R₁; t is 0-2; W₃, W₄, W₅ are independently O, S(═O)r where r is 0-2, CR₄R₄, or N—R₁; with the proviso that whenever W2 is S or O it is not adjacent to a W1, W2 or W3 that is also S or O; u is 1-3; and one of Z₁ or Z₃ is a carbon atom to which the remainder of the molecule is attached; or

Z₃ is N; Z₁, Z₂, and Z₄ are independently CR₂; Y₁ and Y₂ are C; Y₃ and Y₄ are independently CH or N; W₁ is O, S(═O)r where r is 0-2, CR₄R₄, N—R₁, or a bond; W₂ is O, S(═O)r where r is 0-2, CR₄R₄, or N—R₁; t is 0-2; W₃, W₄, and W₅ are independently O, S(═O)r where r is 0-2, CR₄R₄, or N—R₁; with the proviso that whenever W2 is S or O it is not adjacent to a W1, W2 or W3 that is also S or O; u is 1-3; and any one of Z₁, Z₂, and Z₄ is a carbon atom to which the remainder of the molecule is attached; or

Z₃ and Z₄ are N; Z₁ and Z₂ are independently CR₂; Y₁ and Y₂ are C; Y₃ and Y₄ are independently CH or N; W₁ is O, S(═O)r where r is 0-2, CR₄R₄, N—R₁, or a bond; W₂ is O, S(═O)r where r is 0-2, CR₄R₄, or N—R₁; t is 0-2; W₃, W₄, and W₅ are independently O, S(═O)r where r is 0-2, CR₄R₄, or N—R₁; with the proviso that whenever W2 is S or O it is not adjacent to a W1, W2 or W3 that is also S or O; u is 1-3; and one of Z₁ or Z₂ is a carbon atom to which the remainder of the molecule is attached; or

Z₄ is N; Z₁, Z₂, and Z₃ are independently CR₂; Y₁ and Y₂ are C; Y₃ and Y₄ are independently CH or N; W₁ is O, S(═O)r where r is 0-2, CR₄R₄, N—R₁, or a bond; W₂ is O, S(═O)r where r is 0-2, CR₄R₄, or N—R₁; t is 0-2; W₃, W₄, and W₅ are independently O, S(═O)r where r is 0-2, CR₄R₄, or N—R₁; with the proviso that whenever W2 is S or O it is not adjacent to a W1, W2 or W3 that is also S or O; u is 1-3; and any one of Z₁, Z₂, and Z₃ is a carbon atom to which the remainder of the molecule is attached; or

In Some Embodiments of Formula 16-B

Z₁ is N; Z₂, Z₃, and Z₄ are independently CR₂; Y₁ and Y₂ are C; Y₃ and Y₄ are independently CH or N; W₁ is O, S(═O)r where r is 0-2, CR₄R₄, or N—R₁; W₂, W₃, and W₄ are independently O, S(═O)r where r is 0-2, CR₄R₄, or N—R₁; with the proviso that whenever W2 is S or O it is not adjacent to a W1, W2 or W3 that is also S or O; t is 1-2; u is 1-3; and any one of Z₂, Z₃, and Z₄ is a carbon atom to which the remainder of the molecule is attached; or

Z₁ and Z₂ are N; Z₃ and Z₄ are independently CR₂; Y₁ and Y₂ are C; Y₃ and Y₄ are independently CH or N; W₁ is O, S(═O)r where r is 0-2, CR₄R₄, or N—R₁; W₂, W₃, and W₄ are independently O, S(═O)r where r is 0-2, CR₄R₄, or N—R₁; with the proviso that whenever W₂ is S or O it is not adjacent to a W1, W2 or W3 that is also S or O; t is 1-2; u is 1-3; and one of Z₃ and Z₄ is a carbon atom to which the remainder of the molecule is attached; or

Z₁ and Z₃ are N; Z₂ and Z₄ are independently CR₂; Y₁ and Y₂ are C; Y₃ and Y₄ are independently CH or N; W₁ is O, S(═O)r where r is 0-2, CR₄R₄, or N—R₁; W₂, W₃, and W₄ are independently O, S(═O)r where r is 0-2, CR₄R₄, or N—R₁; with the proviso that whenever W₂ is S or O it is not adjacent to a W1 , W₂ or W3 that is also S or O; t is 1-2; u is 1-3; and one of Z₂ or Z₄ is a carbon atom to which the remainder of the molecule is attached; or

Z₁ and Z₄ are N; Z₂ and Z₃ are independently CR₂; Y₁ and Y₂ are C; Y₃ and Y₄ are independently CH or N; W₁ is O, S(═O)r where r is 0-2, CR₄R₄, or N—R₁; W₂, W₃, and W₄ are independently O, S(═O)r where r is 0-2, CR₄R₄, or N—R₁; with the proviso that whenever W₂ is S or O it is not adjacent to a W1, W₂ or W3 that is also S or O; t is 1-2; u is 1-3; and one of Z₂ and Z₃ is a carbon atom to which the remainder of the molecule is attached; or

Z₂ is N; Z₁, Z₃ and Z₄ are independently CR₂; Y₁ and Y₂ are C; Y₃ and Y₄ are independently CH or N; W₁ is O, S(═O)r where r is 0-2, CR₄R₄, or N—R₁; W₂, W₃, and W₄ are independently O, S(═O)r where r is 0-2, CR₄R₄, or N—R₁; with the proviso that whenever W₂ is S or O it is not adjacent to a W1, W₂ or W3 that is also S or O; t is 1-2; u is 1-3; and any one of Z₁, Z₃, and Z₄ is a carbon atom to which the remainder of the molecule is attached; or

Z₂ and Z₃ are N; Z₁ and Z₄ are independently CR₂; Y₁ and Y₂ are C; Y₃ and Y₄ are independently CH or N; W₁ is O, S(═O)r where r is 0-2, CR₄R₄, or N—R₁; W₂, W₃, and W₄ are independently O, S(═O)r where r is 0-2, CR₄R₄, or N—R₁; with the proviso that whenever W₂ is S or O it is not adjacent to a W1, W₂ or W3 that is also S or O; t is 1-2; u is 1-3; and one of Z₁ or Z₄ is a carbon atom to which the remainder of the molecule is attached; or

Z₂ and Z₄ are N; Z₁ and Z₃ are independently CR₂; Y₁ and Y₂ are C; Y₃ and Y₄ are independently CH or N; W₁ is O, S(═O)r where r is 0-2, CR₄R₄, or N—R₁; W₂, W₃, and W₄ are independently O, S(═O)r where r is 0-2, CR₄R₄, or N—R₁; with the proviso that whenever W₂ is S or O it is not adjacent to a W1, W₂ or W3 that is also S or O; t is 1-2; u is 1-3; and one of Z₁ or Z₃ is a carbon atom to which the remainder of the molecule is attached; or

Z₃ is N; Z₁, Z₂, and Z₄ are independently CR₂; Yₗ and Y2 are C; Y₃ and Y₄ are independently CH or N; W₁ is O, S(═O)r where r is 0-2, CR₄R₄, or N—R₁; W₂, W₃, and W₄ are independently O, S(═O)r where r is 0-2, CR₄R₄, or N—R₁; with the proviso that whenever W₂ is S or O it is not adjacent to a W1, W₂ or W3 that is also S or O; t is 1-2; u is 1-3; and any one of Z₁, Z₂, and Z₄ of is a carbon atom to which the remainder of the molecule is attached; or

Z₃ and Z₄ are N; Z₁ and Z₂ are independently CR₂; Y₁ and Y₂ are C; Y₃ and Y₄ are independently CH or N; W₁ is O, S(═O)r where r is 0-2, CR₄R₄, or N—R₁; W₂, W₃, and W₄ are independently O, S(═O)r where r is 0-2, CR₄R₄, or N—R₁; with the proviso that whenever W₂ is S or O it is not adjacent to a W1, W₂ or W3 that is also S or O; t is 1-2; u is 1-3; and one of Z₁ or Z₂ is a carbon atom to which the remainder of the molecule is attached; or Z₄ is N; Z₁, Z₂, and Z₃ are independently CR₂; Y₁ and Y₂ are C; Y₃ and Y₄ are independently CH or N; W₁ is O, S(═O)r where r is 0-2, CR₄R₄, or N—R₁; W₂, W₃, and W₄ are independently O, S(═O)r where r is 0-2, CR₄R₄, or N—R₁; with the proviso that whenever W₂ is S or O it is not adjacent to a W1, W₂ or W3 that is also S or O; t is 1-2; u is 1-3; and any one Z₁, Z₂, and Z₃ is a carbon atom to which the remainder of the molecule is attached; or

In Some Embodiments of Formula 16-C:

Z₁, Z₂, and Z₃ are independently CR₂; Y₁, Y₂, and Y₄ are C; Y₂ is N; W₁, W₂, and W₄ are independently O, S(═O)r where r is 0-2, CR₄R₄, or N—R₁; with the proviso that whenever W₂ is S or O it is not adjacent to a W1, W₂ or W3 that is also S or O; t is 1-2; u is 1-2; and any one of Z₁, Z₂, and Z₃ is a carbon atom to which the remainder of the molecule is attached; or

Z₁ is N; Z₂ and Z₃ are independently CR₂; Y₁, Y₂, and Y₄ are C; Y₂ is N; W₁, W₂, and W₄ are independently O, S(═O)r where r is 0-2, CR₄R₄, or N—R₁; with the proviso that whenever W₂ is S or O it is not adjacent to a W1, W₂ or W3 that is also S or O; t is 1-2; u is 1-2; and one of Z₂ or Z₃ is a carbon atom to which the remainder of the molecule is attached; or

Z₁ and Z₃ are N; Z₃ is CR₂; Y₁, Y₂, and Y₄ are C; Y₂ is N, W₁, W₂, and W₄ are independently O, S(═O)r where r is 0-2, CR₄R₄, or N—R₁; with the proviso that whenever W₂ is S or O it is not adjacent to a W1, W₂ or W3 that is also S or O; t is 1-2; u is 1-2; and Z₃ is a carbon to which the remainder of the molecule is attached; and

R₁ is H, C₁₋₆alkyl, -aryl, -heteroaryl or mono or bicyclic saturated heterocyclyl having 1-3 heteroatoms independently selected from O, N or S, C₃₋₇cycloalkyl, C₂₋₈alkenyl, C₂₋₆alkynyl with the proviso that both the double bond and the triple bond should not be present at a carbon which is directly linked to N; C₁₋₆perfluoroalkyl, —S(═O)ₚ where p is 2, —C(═O)heteroaryl, —C(═O)(C₆₋₁₄aryl), —C(═O) (C₁₋₆alkyl), —C(═O)(C₃₋₆cycloalkyl), —C(═O) mono or bicyclic saturated heterocyclyl having 1-3 heteroatoms independently selected from O, N or S, C₁₋₆alkyl aryl, (C₁₋₆alkyl)heteroaryl, aryl(C₁₋₆alkyl), heteroaryl(C₁₋₆alkyl), C₁₋₆alkyl mono or bicyclic saturated heterocyclyl having 1-3 heteroatoms independently selected from O, N or S, arylalkenyl of 8 to 16 carbon atoms, —CONR₆R₇, —SO₂NR₆R₇, arylalkyloxyalkyl, (C₁₋₆alkyl)O(C₁₋₆alkyl)-aryl, (C₁₋₆alkyl)-O-(C₁₋₆alkyl) heteroaryl, aryloxyalkyl, heteroaryloxyalkyl, aryloxyaryl, aryloxyheteroaryl, alkylaryloxyaryl, alkylaryloxyheteroaryl, alkylaryloxyalkylamines, C₁₋₆alkoxy carbonyl, aryloxy carbonyl, heteroaryloxy carbonyl. Some R₁ groups can be H, C₁₋₆alkyl, aryl, —C(═O)(C₁₋₆alkyl), C₂₋₈alkenyl, C₂₋₆alkynyl, C₃₋₇cycloalkyl, SO₂alkyl, SO₂aryl, heterocyclyl, —CONR₆R₇, and heteroaryl.

R₂ is hydrogen, C₁₋₆alkyl, C₂₋₈alkenyl having 1 to 2 double bonds, C₂₋₆alkynyl having 1 to 2 triple bonds, halogen, cyano, N—R₆R₇, C₁₋₆alkoxy, hydroxy; aryl, heteroaryl, COOR₆, C₁₋₆alkyl aryloxy alkylamines, aryloxy, heteroaryloxy, alkenyloxy, C₂₋₆alkynyloxy, C₁₋₆alkylamino(C₁₋₆alkoxy), alkylene dioxy, aryloxy-C₁₋₆alkyl amine, C₁₋₆perfluoro alkyl, S(═O)_{q}—C₁₋₆alkyl, S(═O)_{q}-aryl where q is 0, 1 or 2, CONR₆R₇, guanidino or cyclic guanidino, C₁₋₆alkylaryl, arylC₁₋₆alkyl, C₁₋₆alkylheteroaryl, heteroaryl-C₁₋₆alkyl, C₁₋₆alkyl mono or bicyclic saturated heterocyclyl having 1-3 heteroatoms independently selected from O, N or S, arylalkenyl of 8 to ₁6 carbon atoms, SO₂NR₆R₇, arylalkyloxyalkyl, aryloxyalkyl, heteroaryloxyalkyl, aryloxyaryl, aryloxyheteroaryl, heteroaryloxyaryl, C₁₋₆alkyl aryloxyaryl, C₁₋₆ alkylaryloxyheteroaryl, aryloxyalkyl, heteroaryloxyalkyl, alkylaryloxyalkylamines, C₃₋₇cycloalkyl, C₃₋₇ saturated or partially saturated heterocycle. Some R₂ groups are H, C₁₋₆alkyl, C₁₋₆alkoxy, heteroaryl, halogen, CN, hydroxy, heterocycle, —CONR₆R₇, COOR₆, aryl, S(═O)_{q}-alkyl, and S(═O)_{q}-aryl;

R₄ is H, optionally substituted C₁₋₆alkyl, and any one of R₄ can be OH, C₁₋₆alkoxy, —S—C₁₋₆alkyl, COOR₆, —NR₆R₇, —CONR₆R₇; or R₄R₄ may together be (═O); or R₄R₄ together with the carbon to which they are attached may form a spiro system of five to eight members with or without the presence of heteroatoms selected from N, O, S(═O)n where n is 0-2, N—R₁;

R₆ and R₇ are independently H, or a group selected from C₁₋₆alkyl, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, alkylaryl, arylalkyl, heteroarylalkyl, C₁₋₆alkylheteroaryl, said group being optionally substituted with nitro, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl-, C₁₋₆alkoxy, -alkoxyalkyl, alkyl-O—C₂₋₄alkyl-O-cyano, -halogen, -hydroxy, —NR₆R₇, —COON, —COO-alkyl, -trifluoromethyl, -trifluoromethoxy, arylalkyl, alkylaryl, R₆R₇N-alkyl-, HO—C₁₋₆alkyl, alkoxyalkyl-, C₁₋₆alkyl-S—, —SO₂NHR₆, —CO₂H, CONR₆R₇, C₆₋₁₄aryl-O—, heteroaryl-O—, —S(═O)ₛ-aryl, wherein s is 0-2, -alkyl-O-alkyl-NR₆R₇, -alkyl-aryl-O-alkyl-N—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl, wherein S is 0-2, or R₆ and R₇ can together with the nitrogen to which they are attached form a 3 to 7 membered saturated ring system optionally having one or two heteroatoms selected from N—R₁, O, and S(═O)ₙ, where n is 0-2.

Some compounds of the present teachings are:

TABLE 3

| | | |
|---|---|---|
| Compound 1 | (5R), (6E)-6-(2,3-dihydro- imidazo[2,1-b]thiazol-5- ylmethylene)-7-oxo-1-aza- bicyclo[3.2.0]hept-2-ene-2- carboxylic acid, sodium salt | 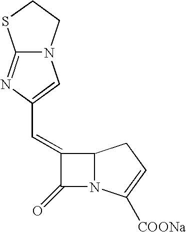 |
| Compound 2 | (5R), (6E)-6-[(5-benzyl-4,5,6,7- tetrahydrothieno[3,2-c]pyridin-2- yl)methylene]-7oxo-1- azabicyclo[3.2.0]hept-2-ene-2- carboxylic acid, sodium salt. | 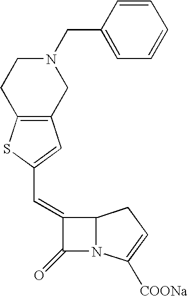 |
| Compound 3 | (5R), (6E)-6-(7-methyl-5,6,7,8- tetrahydroimidazo[1,2-a]pyrazin- 2-ylmethylene)-7-oxo-1-aza- bicyclo[3.2.0]hept-2-ene-2- carboxylic acid, sodium salt. | 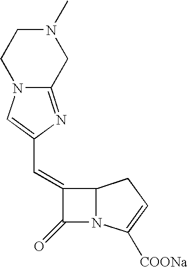 |
| Compound 4 | (5R), (6E)-7-oxo-6-(5,6,7,8- tetrahydroimidazo[1,2-a]pyrazin- 2-ylmethylene)-1- azabicyclo[3.2.0]hept-2-ene-2- carboxylic acid, sodium salt. | 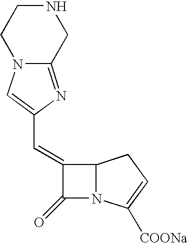 |
| Compound 5 | (5R), (6E)-6-{[5-(4- methoxybenzyl)-4,5,6,7- tetrahydrothieno[3,2-c]pyridin-2- yl)]methylene}-7-oxo-1- azabicyclo[3.2.0]hept-2-ene-2- carboxylic acid, sodium salt. | 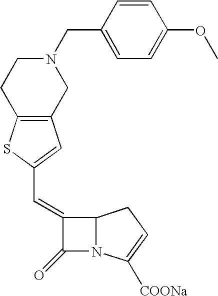 |
| Compound 6 | (5R), (6E)-6-(5,6-dihydro-8H- imidazo[2,1-c][1,4]thiazin-2- ylmethylene)-7-oxo-1- azabicyclo[3.2.0]hept-2-ene-2- carboxylic acid, sodium salt | 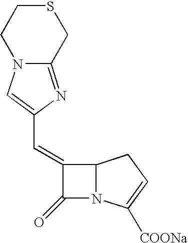 |
| Compound 7 | (5R), (6E)-6-(6,7-dihydro-5H- pyrrolo[1,2-a]imidazol-2- ylmethylene)-7-oxo-1-aza- bicyclo[3.2.0]hept-2-ene-2- carboxylic acid, sodium salt | 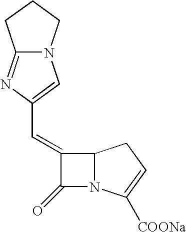 |
| Compound 8 | (5R), (6E)-6-(5,6-dihydro-8H- imidazo[2,1-c][1,4]oxazin-2- ylmethylene)-7-oxo-1- azabicyclo[3.2.0]hept-2-ene-2- carboxylic acid, sodium salt | 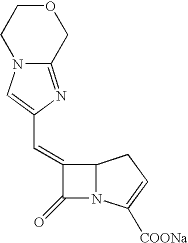 |
| Compound 9 | (5R), (6E)-6-(5,6-dihydro-4H- pyrrolo[1,2-b]pyrazol-2- ylmethylene)-7-oxo-1- azabicyclo[3.2.0]hept-2-ene-2- carboxylic acid, sodium salt | 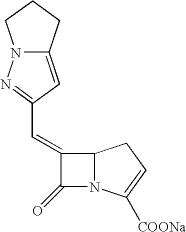 |
| Compound 10 | (5R), (6E)-7-oxo-6-(4,5,6,7- tetrahydropyrazolo[1,5-a]pyridin- 2-ylmethylene)-1- azabicyclo[3.2.0]hept-2-ene-2- carboxylic acid, sodium salt | 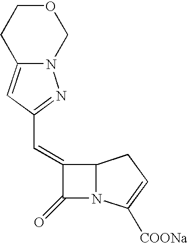 |
| Compound 11 | (5R), (6E)-6-(7-methyl-6-oxo- 5,6,7,8-tetrahydro-imidazo[1,2- a]pyrazin-2-ylmethylene)-7-oxo- 1-aza-bicyclo[3.2.0]hept-2-ene-2- carboxylic acid sodium salt | 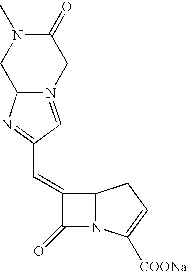 |
| Compound 12 | (5R), (6E)-6-(6,7-dihydro-4H- pyrazolo[5,1-c][1,4]thiazin-2- ylmethylene)-7-oxo-1- azabicyclo[3.2.0]hept-2-ene-2- carboxylic acid, sodium salt | 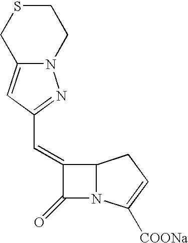 |
| Compound 13 | (5R), (6E)-7-Oxo-6-(4H-5-thia- 1,6a-diazapentalen-2- ylmethylene)-1- azabicyclo[3.2.0]hept-2-ene-2- carboxylic acid, sodium salt | 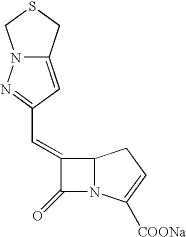 |
| Compound 14 | (5R), (6E)-6-(7H-imidazo[1,2- c]thiazol-2-ylmethylene)-7-oxo-1- azabicyclo[3.2.0]hept-2-ene-2- carboxylic acid, sodium salt | 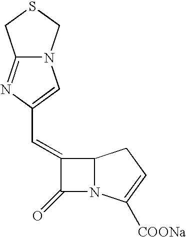 |
| Compound 15 | (5R), (6E)-7-oxo-6-[(4-oxo-6,7- dihydro-4H-pyrazolo[5,1- c][1,4]oxazin-2-yl)methylene]-1- azabicyclo[3.2.0]hept-2-ene-2- carboxylic acid | 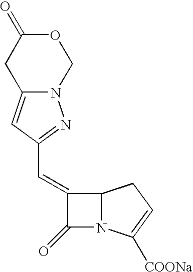 |
| Compound 16 | 6-(6,7-dihydro-4H-thieno[3,2- c]pyran-2-ylmethylene)-7-oxo-1- aza-bicyclo[3.2.0]hept-2-ene-2- carboxylic acid | 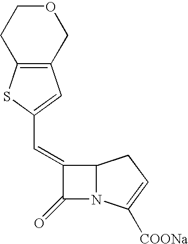 |
| Compound 17 | 6-(6,7-dihydro-4H-thieno[3,2- c]thiopyran-2-ylmethylene)-7- oxo-1-aza-bicyclo[3.2.0]hept-2- ene-2-carboxylic acid | 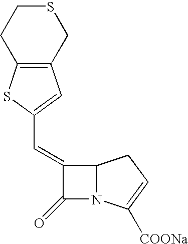 |
| Compound 18 | 6-(5-methyl-4,5,6,7-tetrahydro- thieno[3,2-c]pyridin-2- ylmethylene)-7-oxo-1-aza- bicyclo[3.2.0]hept-2-ene-2- carboxylic acid | 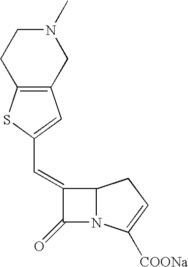 |
| Compound 19 | 2-(2-carboxy-7-oxo-1-aza- bicyclo[3.2.0]hept-2-en-6- ylidenemethyl)-6,7-dihydro-4H- thieno[3,2-c]pyridine-5-carboxylic acid ethyl ester | 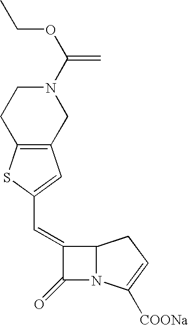 |
| Compound 20 | 7-oxo-6-(6,7,8,9-tetrahydro-5H- imidazo[1,2-a]azepin-2- ylmethylene)-1-aza- bicyclo[3.2.0]hept-2-ene-2- carboxylic acid | 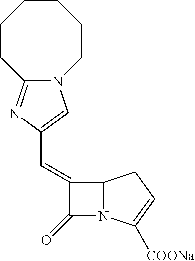 |
| Compound 21 | (5R), (6E)-6-(7-benzyl-5,6,7,8- tetrahydroimidazo[1,2-a]pyrazin- 2-ylmethylene)-7-oxo-1- azabicyclo[3.2.0]hept-2-ene-2- carboxylic acid | 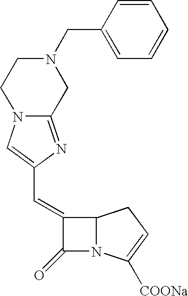 |
| Compound 22 | (5R), (6E)-7-oxo-6-{[5-(pyridin-3- ylmethyl)-4,5,6,7- tetrahydrothieno[3,2-c]pyridin-2- yl)]methylene}-7oxo-1- azabicyclo[3.2.0]hept-2-ene-2- carboxylic acid | 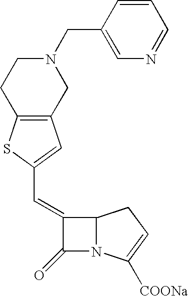 |
| Compound 23 | (5R), (6E)-7-oxo-6-{[5-(pyridin-3- ylcarbonyl)-4,5,6,7- tetrahydrothieno[3,2-c]pyridin-2- yl)]methylene}-7oxo-1- azabicyclo[3.2.0]hept-2-ene-2- carboxylic acid | 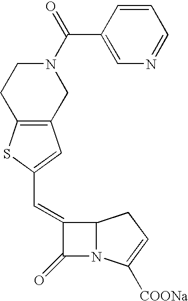 |
| Compound 24 | (5R), (6E)-7-oxo-6-{[5- (phenylacetyl)-4,5,6,7- tetrahydrothieno[3,2-c]pyridin-2- yl)]methylene}-7oxo-1- azabicyclo[3.2.0]hept-2-ene-2- carboxylic acid | 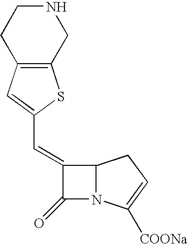 |
| Compound 25 | (5R), (6E)-6-(6,7-dihydro-5H- cyclopenta[d]imidazo[2,1- b][1,3]thiazol-2-ylmethylene)-7- oxo-1-azabicyclo[3.2.0]hept-2- ene-2-carboxylic acid | 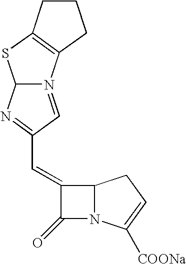 |
| Compound 26 | (5R), (6E)-7-oxo-6-(5,6,7,8- tetrahydroimidazo[2,1- b][1,3]benzothiazol-2- ylmethylene)-1- azabicyclo[3.2.0]hept-2-ene-2- carboxylic acid sodium salt | 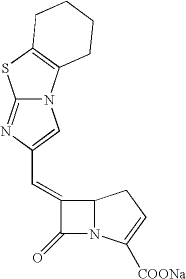 |
| Compound 27 | (5R), (6E)-6-(5,8-dihydro-6H- imidazo[2,1-b]pyrano[4,3- d][1,3]thiazol-2-ylmethylene)-7- oxo-1-azabicyclo[3.2.0]hept-2- ene-2-carboxylic acid | 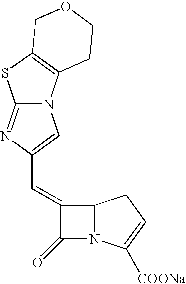 |
| Compound 28 | (5R), (6E)-6-(4,5,6,7-tetrahydro- 1,3a,3b,8-tetraaza- cyclopenta[a]indene-2- ylmethylene)-7-oxo-1-aza- bicyclo[3.2.0]hept-2-ene-2- carboxylic acid sodium salt | 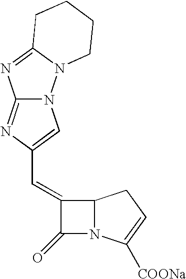 |
| Compound 29 | (5R), (6E)-6-(5H-Imidazo[2,1- a]isoindol-2-ylmethylene)-7-oxo- 1-aza-bicyclo[3.2.0]hept-2-ene- 2-carboxylic acid sodium salt | 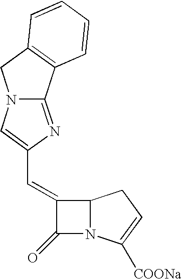 |
| Compound 30 | (5R), (6E)-7-oxo-6-(5,6,7,8- tetrahydropyrazolo[5,1- b][1,3]benzoxazol-2-ylmethylene)- 1-azabicyclo[3.2.0]hept-2-ene-2- carboxylic acid, sodium salt | 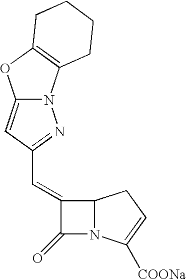 |
| Compound 31 | (5R), (6E)-6-(7,8-dihydro-5H- pyrano[4,3-d]pyrazolo[5,1- b][1,3]oxazol-2-ylmethylene)7- oxo-1-azabicyclo[3.2.0]hept-2- ene-2-carboxylic acid, sodium salt and (the E isomer) | 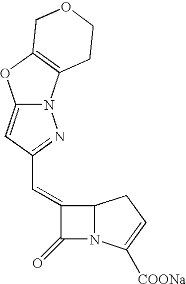 |
| Compound 32 | (5R), (6E)-6-{[6- (ethoxycarbonyl)-5,6,7,8- tetrahydropyrazolo [5′,1′:2,3][1,3]oxazolo[5,4- c]pyridin-2-yl]methylene}-7-oxo- 1-azabicyclo[3.2.0]hept-2-ene-2- carboxylic acid, sodium salt | 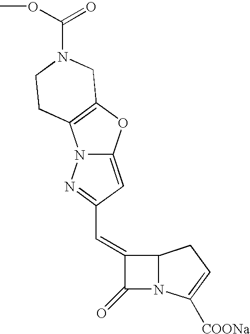 |
| Compound 33 | (5R), (6E)-6-({5-[2- (benzyloxy)ethoxy]-7,8-dihydro- 6H-cyclopenta[e]imidazo[1,2- a]pyrimidin-2-yl}methylene)-7- oxo-1-azabicyclo[3.2.0]hept-2- ene-2-carboxylic acid, sodium salt | 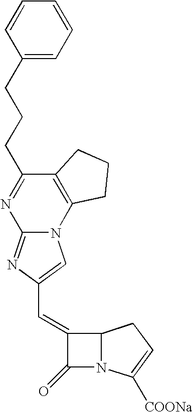 |
| Compound 34 | (5R), (6E)-6-[(5-methoxy-7,8- dihydro-6H- cyclopenta[e]imidazo[1,2- a]pyrimidin-2-yl)methylene]-7- oxo-1-azabicyclo[3.2.0]hept-2- ene-2-carboxylic acid, sodium salt | 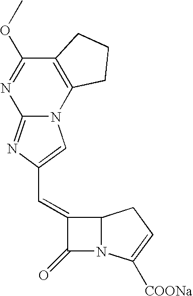 |
| Compound 35 | (5R), (6E)-6-imidazo[1,2- a]quinolin-2-ylmethylene-7-oxo- 1-azabicyclo[3.2.0]hept-2-ene-2- carboxylic acid, sodium salt | 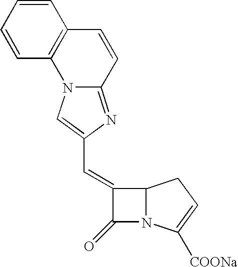 |
| Compound 36 | (5R), (6E)-6-(imidazo[1,2- a]quinoxaline-2-ylmethylene)-7- oxo-1-azabicyclo[3.2.0] hepto-2- ene-2-carboxylic acid, sodium salt | 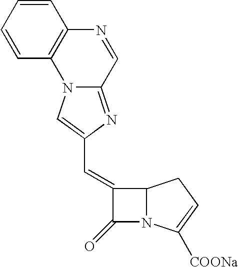 |
| Compound 37 | (5R), (6E)-6-(imidazo[2,1- b]benzothiazol-7-ylmethylene)-7- oxo-1-azabicyclo[3.2.0]hept-2- ene-2-carboxylic acid, sodium salt | 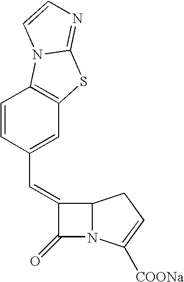 |
| Compound 38 | (5R), (6E)-6-(2,3- dihydro[1,3]thiazolo[3,2- a]benzimidazol-6-ylmethylene)-7- oxo-1-azabicyclo[3.2.0]hept-2- ene-2-carboxylic acid, sodium salt | 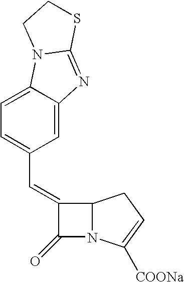 |
| Compound 39 | (5R), (6E)-7-oxo-6- ([1,3]thiazolo[3,2-a]benzimidazol- 6-ylmethylene)-1- azabicyclo[3.2.0]hept-2-ene-2- carboxylic acid, sodium salt | 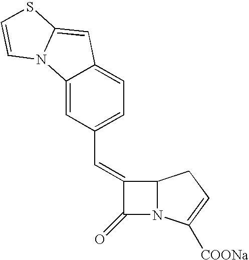 |
| Compound 40 | (5R), (6E)-6-(3,4-dihydro-2H- [1,3]thiazino[3,2-a]benzimidazol- 7-ylmethylene)-7-oxo-1- azabicyclo[3.2.0]hept-2-ene-2- carboxylic acid, sodium salt | 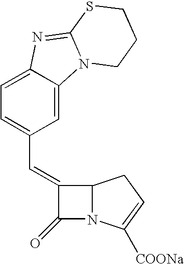 |
| Compound 41 | (5R), (6E)-7-oxo-6- ([1,3]thiazolo[3,2-a]benzimidazol- 2-ylmethylene)-1-1- azabicyclo[3.2.0]hept-2-ene-2- carboxylic acid, sodium salt | 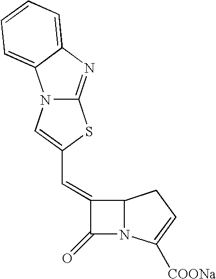 |
| Compound 42 | (5R), (6E)-7-oxo-6-(4H- thieno[2′,3′:4,5]thiopyrano[2,3- b]pyridin-2-ylmethylene)-1- azabicyclo[3.2.0]hept-2-ene-2- carboxylic acid , sodium salt | 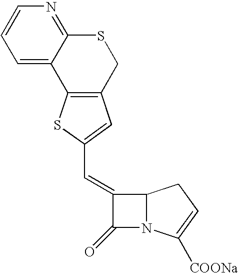 |
| Compound 43 | (5R), (6E)-8-[(9-methyl-9H- imidazo[1,2-a]benzimidazol-2- yl)methylene]-7-oxo-1- azabicyclo[3.2.0]hept-2-ene-2- carboxylic acid, sodium salt | 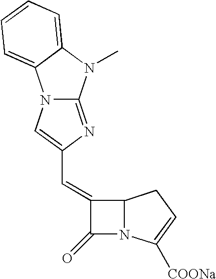 |
| Compound 44 | (5R), (6E)-6-(7,8-dihydro-5H- pyrano[4,3-d]pyrazolo[5,1- b][1,3]oxazol-2-ylmethylene)7- oxo-1-azabicyclo[3.2.0]hept-2- ene-2-carboxylic acid, sodium salt | 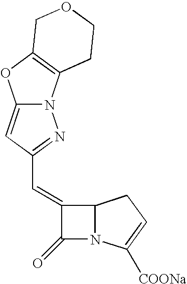 |
| Compound 45 | 6-(5-ethoxy-7,8-dihydro-6H- 3,4,8b-triaza-as-indacen-2- ylmethylene)-7-oxo-1-aza- bicyclo[3.2.0]hept-2-ene-2- carboxylic acid, sodium salt | 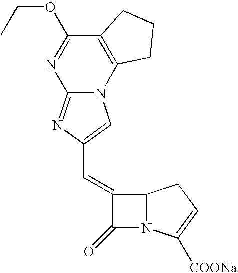 |

Pharmaceutically acceptable salts are those salts that may be administered or provided to a warm-blooded animal, such as, sodium, potassium or calcium alkaline earth metal salts.

A compound's structural formula includes any tautomers, any stereoisomers (except where stereochemistry is clearly noted) and any crystalline forms.

### DEFINITION OF TERMS

As used herein, the term “alkyl” as a group or part of a group is intended to denote hydrocarbon groups including straight chain and branched saturated hydrocarbons. Alkyl groups can contain from 1-20, or 1-12, or 1-6 carbon atoms. Nonlimiting examples of straight chain and branched alkyl groups include methyl (Me), ethyl (Et), propyl (e.g., n-propyl and isopropyl), butyl (e.g., n-butyl, isobutyl, s-butyl, and t-butyl), pentyl groups (e.g., n-pentyl, isopentyl, and neopentyl), hexyl groups, and the like.

The term “cycloalkyl” is intended to mean a monocyclic or bicyclic saturated hydrocarbon group having the indicated number of carbon atoms. For example, a C₃₋₇cycloalkyl group would include cyclopropyl, cyclobutyl, cyclopentyl, cyclohexyl, and cycloheptyl groups, as well as polycyclic systems (e.g., containing fused, bridged, and/or spiro ring systems). Any suitable ring position of a cyclic alkyl group can be covalently linked to the defined chemical structure. Unless otherwise indicated, alkyl groups are unsubstituted. However, where indicated, alkyl groups may be substituted with one or more independently selected substituents as described herein.

As used herein, the term “alkenyl” as a group or part of a group is intended to denote an alkyl group that contains at least one carbon-carbon double bond. Alkenyl groups can contain from 2-20, or 2-12, or 2-8 carbon atoms. Nonlimiting examples of straight chain and branched alkenyl groups include ethenyl, propenyl, butenyl, pentenyl, hexenyl, butadienyl, pentadienyl, hexadienyl, vinyl, allyl, 2-methyl-allyl, 4-but-3-enyl, 4-hex-5-enyl, 3-methyl-but-2-enyl, cyclohex-2-enyl, and the like. The one or more carbon-carbon double bonds can be internal (such as in 2-butene) or terminal (such as in 1-butene). Additionally, hydrocarbon alkenyl moieties may be mono or polyunsaturated, and may exist in the E or Z configurations. The compounds of this invention are meant to include all possible E and Z configurations. Alkenyl groups may be substituted with one or more independently selected substituents as described herein.

The term “cycloalkenyl” is intended to mean a cycloalkyl group that contains at least one carbon-carbon double bond. Examples of cycloalkenyl groups include, but are not limited to, cyclopentenyl, cyclohexenyl, cyclohexadienyl, cycloheptatrienyl, and the like. Alkenyl groups may be substituted with one or more independently selected substituents as described herein. Any suitable ring position of a cycloalkenyl group can be covalently linked to the defined chemical structure. Unless otherwise indicated, alkenyl groups are unsubstituted. However, where indicated, alkenyl groups may be substituted with one or more independently selected substituents as described herein.

As used herein, the term “alkynyl” is intended to denote an alkyl group that contains at least one carbon-carbon triple bond. Alkynyl groups can contain from 2-20, or 2-12, or 2-6, or 2-3 carbon atoms. Examples of alkynyl groups include, but are not limited to, ethynyl, propynyl, butynyl, pentynyl, pent-2-yne, ethynyl-cyclohexyl, and the like. The one or more carbon-carbon triple bonds can be internal (such as in 2-butyne) or terminal (such as in 1-butyne). Alkynyl groups may be substituted with one or more independently selected substituents as described herein.

If alkyl, alkenyl, alkynyl, cycloalkyl, or cycloalkenyl is “optionally substituted”, possible substituents are: nitro, C₆₋₂₀aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl, C₁₋₆alkoxy, (C₁₋₆alkoxy)(C₁₋₆alkyl), (C₁₋₆alkyl)-O-(C₂₋₄alkyl)-O-, cyano, halogen, hydroxy, —N—R₆R₇, —COON, C(═O)O(C₁₋₆alkyl), -trifluoromethyl, -trifluoromethoxy, (C₆₋₁₄aryl)(C₁₋₆alkyl), (C₁₋₆alkyl)(C₆₋₁₄aryl), R₆R₇N—(C₁₋₆alkyl), HO—(C₁₋₆alkyl), (C₁₋₆alkoxy)(C₁₋₆alkyl), (C₁₋₆alkyl)-S—, —SO₂N—R₆R₇, —SO₂NHR₆, —CO₂H, CONR₆R₇, (C₆₋₁₄aryl)O—, heteroaryl-O—, —S(═O)ₛ—(C₆₋₁₄aryl), where s is 0-2, -alkyl-O-alkyl-NR₆R₇, (C₁₋₆alkyl)(C₆₋₁₄aryl)-O—(C₁₋₆alkyl)N—R₆R₇, C₁₋₆alkyl, C₂₋₈alkenyl, C₂₋₆alkynyl, C₃₋₇cycloalkyl, (C₁₋₆alkoxy)(C₁₋₆alkyl)-O—, R₆R₇N—(C₁₋₆alkyl), and —S(═O)ₛ-heteroaryl, where s is 0-2.

As used herein, the term “aryl” as a group or part of a group refers to an aromatic monocyclic hydrocarbon ring system or a polycyclic hydrocarbon ring system (e.g., bicyclic or tricyclic), e.g., of 6-20 carbon atoms where at least any one of the rings present in the ring system is an aromatic hydrocarbon ring. Any suitable ring position of the aryl group can be covalently linked to the defined chemical structure. In some embodiments, an aryl group can have only aromatic rings e.g., phenyl, 1-naphthyl, 2-naphthyl, anthracenyl, phenanthrenyl groups, and the like. In other embodiments, an aryl group can be a polycyclic ring system in which at least one aromatic ring is fused (i.e., having a bond in common with) to one or more cyclic alkyl or heterocyclic alkyl rings, provided that the group is attached to the remainder of the molecule through the aromatic portion thereof. Examples of such aryl groups include, among others, benzo derivatives of cyclopentane (i.e., an indanyl group, which is a 5,6-bicyclic cyclic alkyl/aromatic ring system), cyclohexane (i.e., a tetrahydronaphthyl group, which is a 6,6-bicyclic cyclic alkyl/aromatic ring system), imidazoline (i.e., a benzimidazolinyl group, which is a 5,6-bicyclic heterocyclic alkyl/aromatic ring system), and pyran (i.e., a chromenyl group, which is a 6,6-bicyclic heterocyclic alkyl/aromatic ring system). Other examples of aryl groups include, but are not limited to, benzodioxanyl, benzodioxolyl, chromanyl, indolinyl groups, and the like.

In some embodiments, an aryl group can be substituted with one or more (e.g., up to 4) independently selected substituents as described herein.

As used herein, the terms, “carbocyclyl”, “carbocycle” or “carbocyclic” refer to (1) a non-aromatic cyclic hydrocarbon group having from 3 to 10 ring carbon atoms. In some embodiments (“C₃₋₈ carbocyclyl”), a carbocyclyl group can have from 3 to 8 ring carbon atoms. In some embodiments a carbocyclyl group can have from 3 to 6 ring carbon atoms (“C₃₋₆ carbocyclyl”). Examples of C₃₋₆ carbocyclyl groups include cyclopropyl, cyclobutyl, cyclopentyl, cyclopentenyl, cyclohexyl, cyclohexenyl, cyclohexadienyl and the like. Examples of C₃₋₈ carbocyclyl groups include the aforementioned C₃₋₆ carbocyclyl groups as well as cycloheptyl, cycloheptadienyl, cycloheptatrienyl, cyclooctyl, bicyclo[2.2.1]heptanyl, bicyclo[2.2.2]octanyl and the like. Examples of C₃₋₁₀ carbocyclyl groups include the aforementioned C₃₋₈ carbocyclyl groups as well as octahydro-1H-indenyl, decahydronaphthalenyl, spiro[4.5]decanyl and the like. As the foregoing examples illustrate, in some embodiments a carbocyclyl group can be monocyclic (“monocyclic carbocyclyl”) or bicyclic (e.g., containing a fused, bridged or spiro ring system), and can be saturated or can contain one or more carbon-carbon double or triple bonds. “Carbocyclyl” also refers to (2) a phenyl group; (3) an aryl group (as defined herein); and (4) a 5- or 6-membered heteroaryl group (as defined herein) fused to a monocyclic carbocyclyl group, where the point of attachment is on the carbocyclyl portion of the group. Examples of such carbocyclyl groups include 1,2,3,4-tetrahydronaphthalen-1-yl, 1,2,3,4-tetrahydronaphthalen-2-yl, 2,3-dihydro-1H-inden-1-yl, 2,3-dihydro-1H-inden-2-yl, 1H-inden-1-yl, 5,6,7,8-tetrahydroquinolin-5-yl, 5,6,7,8-tetrahydroquinolin-7-yl, 4,5,6,7-tetrahydro-1H-indol-4-yl, 4,5,6,7-tetrahydro-1H-indol-6-yl, 4,5,6,7-tetrahydrobenzofuran-7-yl and the like.

The term “heterocyclic” or “heterocyclic group” or “heterocycle” is used herein to describe a 3-14 membered monocyclic or polycyclic, ring system having at least 1, and up to 4, ring heteroatoms independently selected from N, O, and S. Heterocyclic groups can be saturated, partially unsaturated, or wholly unsaturated, but cannot be aromatic. When the heterocyclic ring contains nitrogen or sulfur atoms in the backbone of any one of the rings, the nitrogen or sulfur atoms can be oxidized, for example, N-oxides, SO, or SO₂. Heterocyclic groups include, without limitation, oxygen-containing rings, nitrogen-containing rings, sulfur-containing rings, and mixed heteroatom containing rings. Nonlimiting examples of heterocyclic groups include aziridinyl, azetidinyl, 1,4-dioxanyl, hexahydroazepinyl, piperazinyl, piperidinyl, piperazinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl.

Saturated or partially saturated heterocyclyl groups include heterocyclic rings selected from the moieties; aziridinyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl. Some saturated or partially saturated heterocyclyl include: aziridinyl, azetidinyl, 1,4-dioxanyl, hexahydroazepinyl, piperazinyl, piperidinyl, pyrrolidinyl, morpholinyl, thiomorpholinyl, tetrahydroquinolinyl, tetrahydroisoquinolinyl, dihydroimidazolyl, and dihydroisooxazolyl.

C₁₋₆alkyl mono or bicyclic saturated or partially saturated heterocyclyl refers to an alkyl group (straight or branched) of C₁₋₆ attached to a heterocycle (which is defined above) through a carbon atom or a nitrogen atom and the other end of the alkyl chain attached to the rest of the molecule. The term ‘optionally substituted’ refers to unsubstituted or substituted with 1 or 2 substituents present on the alkyl or heterocyclic portion of the molecule, as defined above;

The term “optionally substituted” is also used herein to refer to the optional substitution of one or more protons with a named substituent or substituents.

The term “alkoxy” as used herein refers to a group of formula —O-alkyl. Examples of alkoxy groups include, but are not limited to, methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy, octoxy, prop-2-oxy, but-2-oxy and methylprop-2-oxy.

The term “halogen” refers to Cl, Br, F, and I.

The term perfluoroalkyl is used herein to refer to both straight- and branched-chain saturated aliphatic hydrocarbon groups having at least one carbon atom and two or more fluorine atoms. Examples include CF₃, CH₂CF₃, CF₂CF₃ and CH(CF₃)₂.

Heteroaryl refers to a aromatic heterocyclic ring system (monocyclic or bicyclic) where the heteroaryl moieties can be: (1) furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazoie, benzimidazole, N-methylbenzimidazole, azabenzimidazole, indazole, quinazoline, quinoline, and isoquinoline; (2) a bicyclic aromatic heterocycle where a phenyl, pyridine, pyrimidine or pyridizine ring is: (a) fused to a 6-membered aromatic (unsaturated) heterocyclic ring having one nitrogen atom; (b) fused to a 5 or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N, or S. Some heteroaryl groups are furan, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, quinoline, isoquinoline, and naphthyridine.

If an aryl or heteroaryl is optionally substituted possible substituents are: nitro, C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, alkoxycarbonyl-, -alkoxy, -alkoxy-alkyl, alkyl-O—C₂₋₄alkyl-O—, -cyano, -halogen, -hydroxy, —N—R₆R₇, -trifluoromethyl, -trifluoromethoxy, _{aryl}C₁₋₆alkyl, alkylaryl, R₆R₇N-alkyl-, HO—C₁₋₆alkyl-, alkoxyalkyl-, alkyl-S—, —SO₂N—R₆R₇, —SO₂NHR₆, —CO₂H, CONR₆R₇, C₆₋₁₄aryl-O—, heteroaryl-O—, —S(═O)ₛ-aryl where s is 0-2, -alkyl-O-alkyl-NR₆R₇, -alkyl-aryl-O-alkylN—R₆R₇, C₁₋₆alkyl, C₂₋₈alkenyl, C₂₋₆alkynyl, C₃₋₇ cycloalkyl, alkoxy-alkyl-O—, R₆R₇N—(C₁₋₆alkyl), and —S(═O)ₛ-heteroaryl where s is 0-2; Substituents for C₆₋₂₀aryl and heteroaryl can include: C₁₋₆alkyl, halogen, —N—R₆R₇, trifluoromethyl, -trifluoromethoxy, (C₆₋₂₀aryl)(C₁₋₆alkyl), and (C₁₋₆alkyl)(C₆₋₂₀aryl).

Arylalkyl refers to an aryl ring of 6-20 carbon atoms attached to an alkyl moiety, for example, (C₆₋₂₀aryl)-C₁₋₆alkyl. Arylalkyl moieties include benzyl, 1-phenylethyl, 2-phenylethyl, 3-phenylpropyl, 2-phenylpropyl and the like. The term ‘optionally substituted’ refers to unsubstituted or substituted with 1 or 2 substituents on the C₁₋₆alkyl or C₆₋₁₄aryl moiety as defined above.

Alkylaryl refers to an alkyl chain of 1-6 carbon atoms (straight or branched) attached to an aryl moiety, which is bonded to the rest of the molecule. For example (C₁₋₆alkyl)-C₆₋₂₀aryl. The term ‘optionally substituted’ refers to unsubstituted or substituted with 1 or 2 substituents on the aryl or alkyl moiety as defined above.

Heteroarylalkyl refers to a heteroaryl attached to a straight or branch alkyl chain of 1 to 6 carbon atoms. Heteroary-alkyl moieties include heteroaryl-(CH₂)₁₋₆ and the like. The term ‘optionally substituted’ refers to unsubstituted or substituted with 1 or 2 substituents on the C₁₋₆alkyl or heteroaryl moiety as defined above.

Alkylheteroaryl refers to an alkyl chain of 1-6 carbon atoms (straight or branched) attached to a heteroaryl moiety, which is bonded to the rest of the molecule. For example (C₁₋₆alkyl)-heteroaryl. The term ‘optionally substituted’ refers to unsubstituted or substituted with 1 or 2 substituents on the alkyl or heteroaryl moiety as defined above.

Arylalkyloxyalkyl refers to (C₆₋₂₀aryl)-(C₁₋₆alky)I—O—(C₁₋₆alkyl). The term ‘optionally substituted’ refers to unsubstituted or substituted with 1 or 2 substituents present on the alkyl and/or aryl portions as defined above;

Alkyloxyalkyl refers to (C₁₋₆alkyl)-O—(C₁₋₆alkyl). The term ‘optionally substituted’ refers to unsubstituted or substituted with 1 or 2 substituents present at the alkyl moiety as defined above;

Aryloxyalkyl refers to (C₆₋₂₀aryl)-O—(C₁₋₆alkyl). The term ‘optionally substituted’ refers to unsubstituted or substituted with 1 or 2 substituents present at the alkyl or aryl moiety as defined above;

Heteroarylalkyloxyalkyl refers to heteroaryl-(C₁₋₆alkyl)-O—(C₁₋₆alkyl). The term ‘optionally substituted’ refers to unsubstituted or substituted with 1 or 2 substituents present on the alkyl or heteroaryl moiety as defined above;

Aryloxyaryl refers to (C₆₋₂₀aryl)-O—(C₆₋₂₀aryl). The term ‘optionally substituted’ refers to unsubstituted or substituted with 1 or 2 substituents present on the aryl moiety as defined above;

Aryloxyheteroaryl refers to (C₆₋₂₀aryl)-O-heteroaryl- or —(C₆₋₂₀aryl)-O-heteroaryl; In this definition either the aryl moiety or the heteroaryl moiety can be attached to the remaining portion of the molecule; The term ‘optionally substituted’ refers to unsubstituted or substituted with 1 or 2 substituents present on the aryl moiety or on the heteroaryl moiety as defined above;

Alkylaryloxyaryl refers to (C₆₋₂₀aryl)-O—(C₆₋₂₀aryl)-(C₁₋₆alkyl) wherein the term ‘optionally substituted’ refers to unsubstituted or substituted with 1 or 2 substituents present at the aryl moiety as defined above;

Alkylaryloxyheteroaryl refers to heteroaryl-O—(C₆₋₂₀aryl)-(C₁₋₆alkyl) wherein the term ‘optionally substituted’ refers to unsubstituted or substituted with 1 or 2 substituents present on the aryl moiety or on the heteroaryl moiety as defined above;

Alkylaryloxyalkylamine refers to R₆R₇N—C₁₋₆alkyl-O—(C₆₋₂₀aryl)-(C₁₋₆alkyl); The terms ‘optionally substituted’ refers to unsubstituted or substituted with 1 or 2 substituents present on the alkyl or aryl moiety as defined above; R₆ and R₇ as defined above;

Alkoxycarbonyl refers to (C₁₋₆alkyl)-O—C(═O) wherein the term ‘optionally substituted’ refers to unsubstituted or substituted with 1 or 2 substituents present on the alkyl portion of the alkoxy moiety as defined above;

Aryloxycarbonyl refers to (C₆₋₁₄aryl)-O—C(═O) wherein the term ‘optionally substituted’ refers to unsubstituted or substituted with 1 or 2 substituents present at the aryl moiety as defined above;

Heteroaryloxy carbonyl refers to heteroaryl-O—C(═O) wherein the term ‘optionally substituted’ refers to unsubstituted or substituted with 1 or 2 substituents present at the heteroaryl moiety as defined above;

Alkoxy refers to (C₁₋₆alkyl)-O— wherein the term ‘optionally substituted’ refers to unsubstituted or substituted with 1 or 2 substituents present at the alkyl moiety as defined above;

Aryloxy refers to C₆₋₂₀aryl-O— wherein the term ‘optionally substituted’ refers to unsubstituted or substituted with 1 or 2 substituents present at the aryl moiety as defined above;

Heteroaryloxy refers to heteroaryl-O— wherein the term ‘optionally substituted’ refers to unsubstituted or substituted with 1 or 2 substituents present at the heteroaryl moiety as defined above;

Alkenyloxy refers to (C₂₋₈alkene)-O—; such as, for example, allyl-O—, but-2-ene-O or like moieties; The term ‘optionally substituted’ refers to unsubstituted or substituted with 1 or 2 substituents present at the alkene moiety as defined above, with the proviso that they form no hetero atom such as, for example, O, S or N—R₁ is present on a carbon, which is attached to a double bond;

Alkynyloxy refers to (C₂₋₆alkyne)-O—; Example CH triple bond C—CH₂-O—, or like moieties; The term ‘optionally substituted’ refers to unsubstituted or substituted with 1 or 2 substituents present at the alkyne moiety as defined above, with the proviso that they form no hetero atom such as, for example, O, S or N—R₁ is present on a carbon atom which is attached to a double or triple bond;

Alkylaminoalkoxy refers to R₆R₇N—(C₁₋₆alkyl)-O—(C₁₋₆alkyl), where the terminal alkyl group attached to the oxygen is connected to the rest of the molecule; The terms

R₆ and R₇ are defined above; The term ‘optionally substituted’ refers to unsubstituted or substituted with 1 or 2 substituents present at the alkyl moiety as defined above;

Alkylenedioxy refers to —O—CH₂—O— or —O—(CH₂)₂—O—;

Aryloxyalkylamine refers to R₆R₇N—(C₁₋₆alkyl)-O—(C₆₋₂₀aryl), where the aryl is attached to the rest of the molecule; The term ‘optionally substituted’ refers to unsubstituted or substituted with 1 or 2 substituents present at the alkyl or aryl moiety as defined above;

Arylalkenyl refers to (C₆₋₂₀aryl)(C₂₋₈alkene), with the proviso that they form no hetero atom such as, for example, O, S or N—R₁ is present on a carbon, which is attached to a double bond; The term ‘optionally substituted’ refers to unsubstituted or substituted with 1 or 2 substituents present on the alkene or aryl moiety as defined above;

Heteroaryloxyalkyl refers to heteroaryl-O—(C₁₋₆alkyl) wherein the term ‘optionally substituted’ refers to unsubstituted or substituted with 1 or 2 substituents present at the heteroaryl moiety as defined above;

Heteroaryloxyaryl refers to heteroaryl-O—(C₆₋₂₀aryl), where the aryl moiety is attached to the rest of the molecule; The term ‘optionally substituted’ refers to unsubstituted or substituted with 1 or 2 substituents present at the heteroaryl moiety or the aryl moiety as defined above;

Alkoxy, alkoxyalkyl, alkoxyalkyloxy and alkylthioalkyloxy are moieties wherein the alkyl chain is 1-6 carbon atoms (straight or branched). Aryloxy, heteroaryloxy, arylthio and heteroarylthio are moieties wherein the aryl and heteroaryl groups are as herein before defined. Arylalkyloxy, heteroarylalkyloxy, arylalkylthio and heteroarylalkylthio are moieties wherein the aryl and heteroaryl groups are as herein before defined and wherein the alkyl chain is 1-6 carbons (straight or branched). Aryloxyalkyl, heteroaryloxyalkyl, aryloxyalkyloxy and heteroaryloxyalkyloxy are substituents wherein the alkyl radical is 1-6 carbon atoms.

The term patient as used herein includes, without limitation, a human, mouse, rat, guinea pig, dog, cat, horse, cow, pig, monkey, chimpanzee, baboon, or rhesus. In one embodiment, the patient is a warm-blooded animal. In another embodiment, the patient is a human.

The term effective amount as used herein refers to an amount of a compound or pharmaceutically acceptable salt of the compound that, when administered to a patient, is effective to prevent, partially ameliorate, or to cure, a condition from which the patient suffers or is suspected to suffer.

The term substantially free of its corresponding opposite enantiomer as used herein means that the compound contains no more than about 10% by weight of its corresponding opposite enantiomer. In other embodiments, the compound that is substantially free of its corresponding opposite enantiomer contains no more than about 5%, or no more than about 1%, or no more than about 0.5%, or no more than about 0.1% by weight of its corresponding opposite enantiomer. An enantiomer that is substantially free of its corresponding opposite enantiomer includes a compound that has been isolated and purified or has been prepared substantially free of its corresponding opposite enantiomer.

The term isolated and purified as used herein refers to an isolate that is separate from other components of a reaction mixture or a natural source. In certain embodiments, the isolate contains at least about 50%, or at least about 55%, or at least about 60%, or at least about 65%, at least about 70%, or at least about 75%, or at least about 80%, or at least about 85%, or at least about 90%, or at least about 95%, or at least about 98% of the compound or pharmaceutically acceptable salt of the compound by weight of the isolate.

The term tautomer as used herein refers to compounds produced by the phenomenon wherein a proton of one atom of a molecule shifts to another atom (Jerry March, Advanced Organic Chemistry: Reactions, Mechanisms and Structures, Fourth Edition, John Wiley & Sons 1992, 69-74). Tautomeric forms can be in equilibrium or sterically locked into one form by appropriate substitution.

### PROCESS OF THE INVENTION

Methods of Making Compounds of Formula I

The compounds of formula I can be prepared using a variety of methods starting from commercially available compounds, known compounds, or compounds prepared by known methods. General synthetic routes to many of the compounds are included in the following schemes. It is understood by those skilled in the art that protection and deprotection steps not shown in the Schemes may be required for these syntheses, and that the order of steps may be changed to accommodate functionality in the target molecule See, Greene, T. W. and Wuts, P. G. M., Protective Groups in Organic Chemistry, Second Edition, John Wiley & Sons 1991, 224-276, which is hereby incorporated in its entirety, for protection of carboxyl groups.

In one embodiment, compounds of the general formula I can be prepared by condensing an appropriately substituted aldehyde 4 with a 6-bromo-carbapenem derivative of structure 1 (Scheme 1) in the presence of anhydrous MgBr₂ or MgBr₂:etherate and a base such as, for example, triethylamine, DMAP or DBU, at minus 20° C. to minus 40° C. The intermediate aldol product 5 can be functionalized with acid chlorides or anhydrides to an acetate, triflate or a tosylate 6. Compound 6 can be smoothly converted to the desired product by a reductive elimination process using a metal such as, for example, activated zinc and phosphate buffer at 20° C. to 35° C. at a pH of 6.5 to 8.0. If the protecting group on the carboxylate oxygen is a para-nitrobenzyl group, then the reductive elimination and deprotection can be achieved by a single step. However, if the protecting group is other than a para-nitrobenzyl group, a two step procedure can be followed.

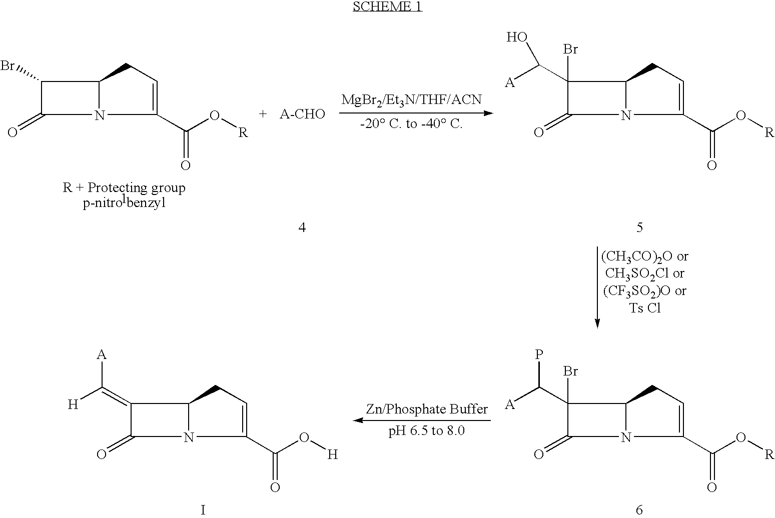

P is O-Acetate; O-Mesylate; O-Triflate; O-Tosylate; Ts is Tosylate

In an alternate procedure, the intermediate 6 can be hydrogenated at 40 pounds per square inch (psi) pressure; in the pressure of 10% pd/C. The product can be isolated as a free acid or as an alkali metal salt. The above-mentioned two-step procedure can be carried out in one step by not isolating intermediate 6. This is a very general, relatively simple and efficient procedure in terms of yield and economic feasibility. This procedure can be adopted to large-scale synthesis and is amenable to a variety of aldehydes.

### Synthesis of an Aldehyde

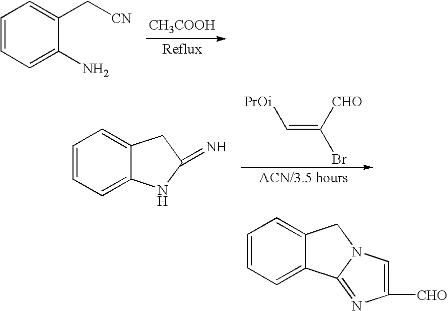

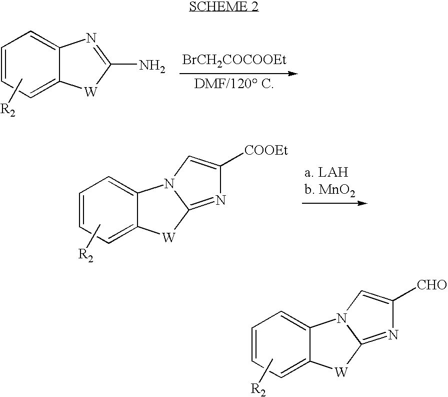

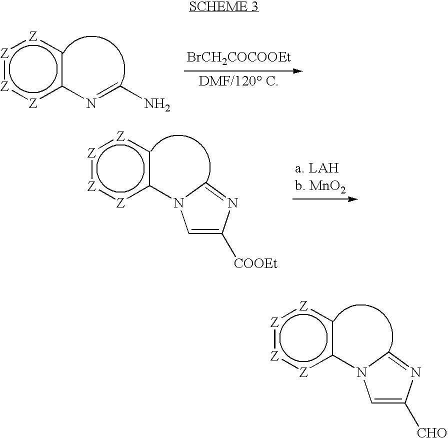

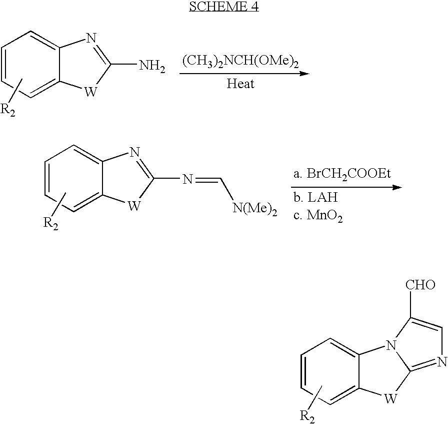

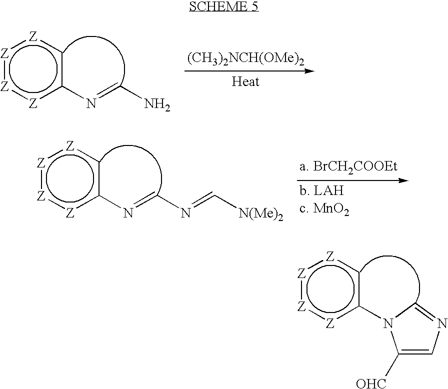

The required aldehydes 4 for the above mentioned transformations can be prepared from their respective alcohol derivatives by MnO₂ oxidation or by Swern oxidation. In some cases the required aldehyde functionality can be introduced directly in the heterocyclic moiety by a Vilsmier Haack reaction using DMF/POCl₃. The aldehydes required for the present investigation may be prepared as depicted in Schemes 2 to 5. This procedure can be adopted to any system where there is an amino functionality adjacent to the —N═ system.

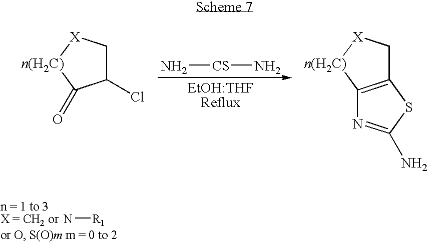

The pyrimidine-condensed aldehyde can be synthesized by following the procedure outlined in Scheme 8. This procedure can be adopted to prepare a variety of condensed tricyclic imidazolo pyrimidine ring systems.

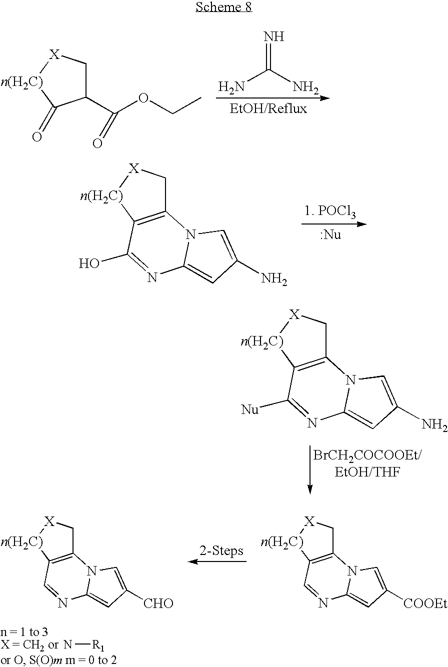

The bicyclic aldehydes 4 required to prepare bicyclic 6-methylidene carbapenem derivatives can be prepared from their respective alcohol derivatives by MnO₂ oxidation or by Swern oxidation. In some cases the required aldehyde functionality can be introduced directly in the heterocyclic moiety by a Vilsmier Haack reaction using DMF/POCl₃. The aldehydes required for the present investigation may be prepared as depicted in Schemes 9 to 12. The N-(tert-butoxycarbonyl)- (i.e. t-Boc protected -4-piperidone), is treated with DMF/POCl₃ to yield 4-chloro-3-formyl derivative. (Scheme 9). This reaction can be conducted on tetrahydro-4H-pyran-4-one and the corresponding tetrahydro-4H-thiopyran-4-one derivative to give the corresponding oxygen and the sulfur derivatives. This reaction can also be conducted on 5- to 8-membered cyclic ketone derivatives. The chloroformyl intermediate can be reacted with 2-mercaptoethylacetate to give the thieno derivative. The ester can be converted to alcohol, which can be converted to the starting aldehyde functionality. Scheme 3 illustrates the preparation of the imidazolo-tetrahydro pyridine derivative and imidazolo pyrazine derivative. 2-aminopyridine or 2-aminopyrazine can be reacted with ethyl bromopyruvate in boiling ethanol to give the cyclized derivative (Scheme 10). Reduction of one ring can be achieved.

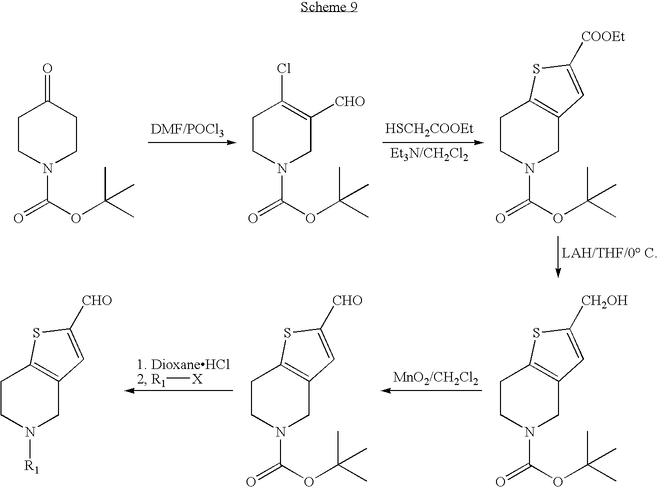

This above mentioned sequence can be conducted starting from tetrahydro-4H-pyran-4-one and the corresponding tetrahydro-4H-thiopyran-4-one. The Vilsmier reaction can be performed on five to eight membered cyclic ketones.

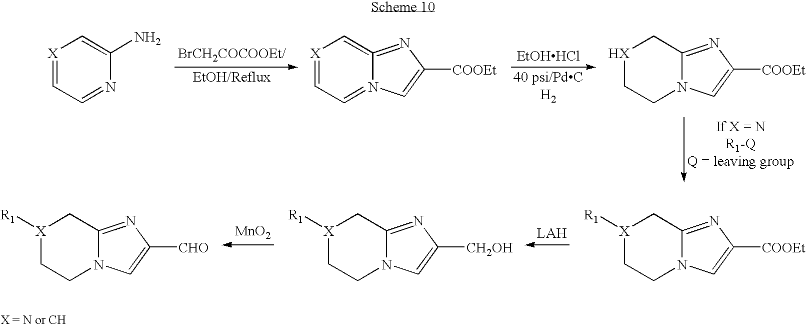

by hydrogenating it over Pd/C under 40 psi pressure in a parr apparatus. Subsequently the ester group can be reduced to alcohol and converted to the aldehyde. In the case of X is N the intermediate amino ester can be derivatized using an appropriate R₁Q (where Q is a leaving group or a condensing group). In the case of Scheme 10, where R₁ is H can be synthesized by the procedure outlined in Scheme 11.

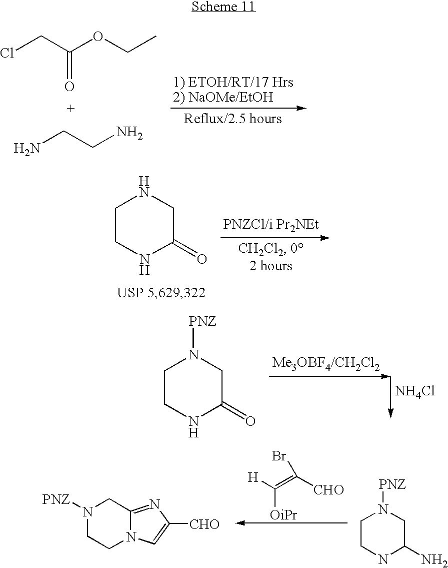

Additional aldehydes may be synthesized as outlined in Schemes 12-14.

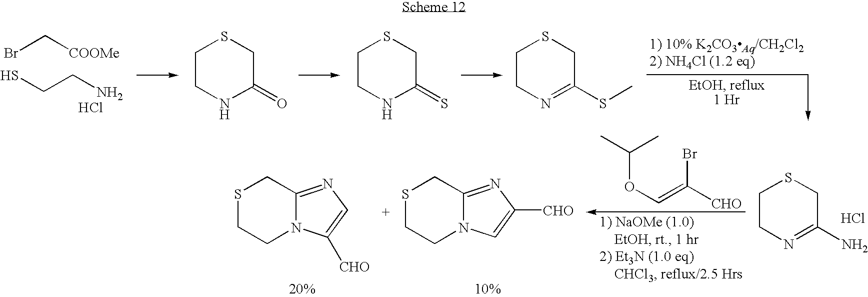

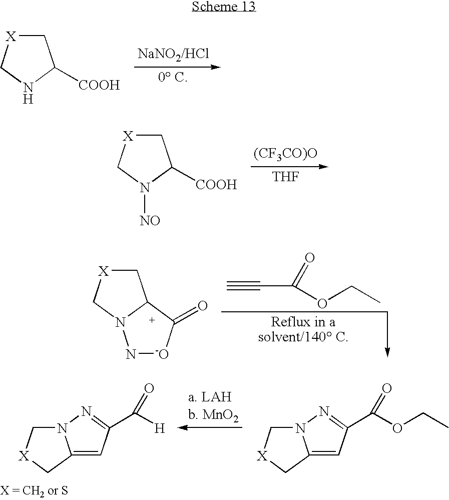

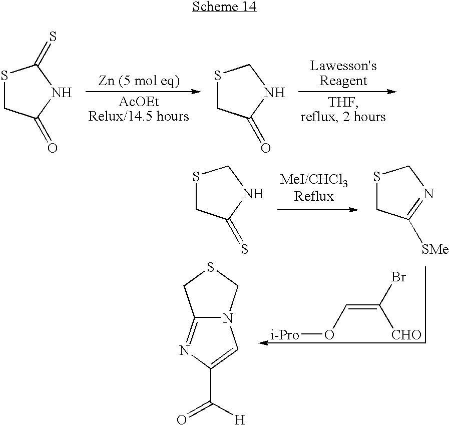

The starting material, compound 1, required for the above mentioned transformations can be prepared by the route outlined in Scheme 15.

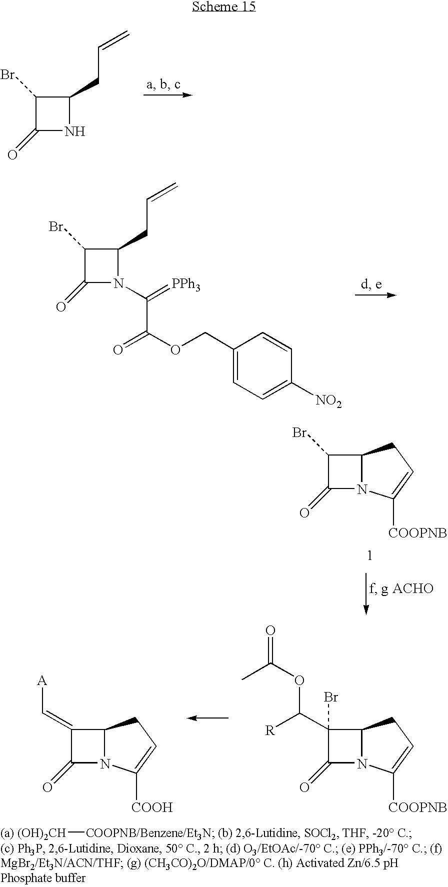

Additional examples of optionally substituted bicyclic heteroaryl groups A and B are set forth in WO 03/093279 A1, WO 03/093277 A1, and US 2004 132708 A1 all of which are incorporated herein by reference in its entirety.

Compounds useful in the present teachings include pharmaceutically acceptable salts or in vivo hydrolysable esters thereof, and as such, the term “compound” as used herein includes a pharmaceutically acceptable salt or in vivo hydrolysable ester thereof. A compound's structural formula also includes any tautomers, any stereoisomers (except where stereochemistry is clearly noted) and any crystalline forms.

In addition, the compounds of formula I can exist as tautomers. Such tautomers can be transient or isolatable as a stable product. These tautomers are within the scope of the present teachings as are the Prodrugs of the compounds of formula I.

Some compounds of the present teachings can contain an asymmetric atom (also referred as a chiral center), and some of the compounds can contain one or more asymmetric atoms or centers, which can thus give rise to optical isomers (enantiomers) and diastereomers (geometric isomers). The present teachings include such optical isomers and diastereomers, as well as, the racemic and resolved, enantiomerically pure R and E stereoisomers, as well as, other mixtures of the R and E stereoisomers and pharmaceutically acceptable salts, hydrates, solvates, metabolites and prodrugs thereof. Optical isomers can be obtained in pure form by standard procedures known to those skilled in the art, and include, but are not limited to, chiral chromatography, diastereomeric salt formation, kinetic resolution, and asymmetric synthesis. The present teachings also encompass cis and trans or E/Z isomers of compounds containing alkenyl moieties (e.g., alkenes and imines). It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

Compounds of the teachings can also include all isotopes of atoms occurring in the intermediates or final compounds. Isotopes include those atoms having the same atomic number but different mass numbers. For example, isotopes of hydrogen include tritium and deuterium.

Therapeutic Administration

In one embodiment, a compound of formula I has β-lactamase inhibitory and antibacterial properties and is useful for the treatment of infections in a patient when combined with cefepime. In another embodiment of the present teachings, a compound of formula I in combination with cefepime provide an effective treatment of a bacterial infection or disease.

In another embodiment, there is provided a method for treating a bacterial infection or disease comprising providing to a patient in need thereof an effective amount of cefepime or a pharmaceutically acceptable salt thereof and a compound of formula I or pharmaceutically acceptable salt or in vivo hydrolysable ester thereof.

The compounds according to the present teachings have β-lactamase inhibitory and antibacterial properties and are useful for the treatment of infections in humans and animals. It should be noted that the compounds of the present teachings, when used in combination with β-lactamase antibiotics will result in the increased antibacterial activity against class-A and class-C producing organisms. β-lactamase antibiotics include penicillin antibiotics such as, for example, piperacillin, amoxycillin, ticarcillin, benzylpenicillins, ampicillin, sulbenicillin, other known penicillin's; cephalosporin such as, for example, cefatrizine, cephaloridine, cephalothin, cefazolin, cephalexin, cephradine, and other known cephalosporins, aztreonam and latamoxef (Moxalactamase). Compounds of the present teachings can be used with piperacillin or amoxicillin, which have a broad spectrum of activity against Gram positive and Gram-negative pathogens.

The compounds of the present teachings may be provided prior to, simultaneously with, or subsequent to a β-lactamase antibiotic (“co-administration”). By “provided”, it is intended to include administering the compound directly or in vivo, e.g. pro-drugs. When the compounds of the present teachings are co-administered with a β-lactamase antibiotic, the ratio of the amount of β-lactamase inhibitor or compound to the amount of the β-lactamase antibiotic may vary 1:1 to 1:100 and can be a ratio of less than 1:10. The composition of the present teachings may be in a form suitable for oral (PO), intravenous (IV) or topical administration. The compositions of the teachings may be in a form of tablets, capsules, creams, syrups, suspension, and sterile solutions suitable for injection or infusion. The compounds of the present teachings can be co-administered, for example, with cefepime orally, with piperacillin intravenously, or amoxicillin intravenously or orally.

In another embodiment, a compound of formula I has β-lactamase inhibitory and antibacterial properties and is useful for the treatment of infections in a patient when combined with a β-lactamase antibiotic. In another embodiment of the present teachings, a compound of formula I in combination with a β-lactamase antibiotic provide an effective treatment of a bacterial infection or disease.

β-lactamase antibiotics as used herein include penicillin antibiotics, cephalosporin antibiotics, and carbapenem antibiotics. For example, penicillin antibiotics such as, for example, carbenicillin, azlocillin, mezlocillin, mecillinam, nafcillin, and oxacillin; cephalosporin antibiotics such as, for example, cefaclor, cefepime, cefamandol, cefdinir, cefditoren, cefatamet, cefixime, cefmetazole, cefotaxime, cefotetan, cefoxitin, cefpodoxime, ceftibuten, ceftizoxime, and cefuroxime; and carbapenem antibiotics such as, for example, loracarbef, imipenem, meropenem, and ertapenem; are useful for the treatment of infections in a patient when combined with a compound of formula I.

In one embodiment, a compound of formula I when used in combination with cefepime results in increased antibacterial activity against a Class-A producing organism. In another embodiment, a compound of formula I when used in combination with cefepime results in increased antibacterial activity against a Class-B producing organism. In another embodiment, a compound of formula I when used in combination with cefepime results in increased antibacterial activity against a Class-C producing organism. In another embodiment, β-compound of formula I when used in combination with cefepime results in increased antibacterial activity against a Class-D producing organism. In another embodiment, a compound of formula I when used in combination with cefepime results in increased antibacterial activity against a Class-A and a Class-C producing organism. In still another embodiment, a compound of formula I when used in combination with cefepime results in increased antibacterial activity against a Class-A, a Class-C, and a Class-D producing organism.

In one embodiment, a compound of formula I when used in combination with a β-lactamase antibiotic results in increased antibacterial activity against a Class-A producing organism. In another embodiment, a compound of formula I when used in combination with a β-lactamase antibiotic results in increased antibacterial activity against a Class-B producing organism. In another embodiment, a compound of formula I when used in combination with a β-lactamase antibiotic results in increased antibacterial activity against a Class-C producing organism. In another embodiment, a compound of formula I when used in combination with a β-lactamase antibiotic results in increased antibacterial activity against a Class-D producing organism. In another embodiment, a compound of formula I when used in combination with a β-lactamase antibiotic results in increased antibacterial activity against a Class-A and a Class-C producing organism. In still another embodiment, a compound of formula I when used in combination with a β-lactamase antibiotic results in increased antibacterial activity against a Class-A, a Class-C, and a Class-D producing organism.

In one embodiment, a compound of formula I and a β-lactamase antibiotic are administered in doses commonly employed when such agents are used individually for the treatment of a bacterial infection or disease.

In another embodiment, a compound of formula I and a β-lactamase antibiotic are administered in doses that are less than the doses commonly employed when such agents are used individually for the treatment of a bacterial infection or disease.

As used herein, a β-lactamase antibiotic includes a pharmaceutically acceptable salt thereof.

In one embodiment, the ratio of β-lactamase antibiotic or pharmaceutically acceptable salt thereof to the compound of formula I or pharmaceutically acceptable salt of in vivo hydrolysable ester thereof is from about 1:1 to about 80:1; 1:1 to about 70:1; 1:1 to about 60:1; from about 1:1 to about 50:1; 1:1 to about 40:1; from about 1:1 to about 30:1; from about 1:1 to about 20:1; from about 1:1 to about 15:1; 1:1 to about 14:1; 1:1 to about 13:1; from about 1:1 to about 12:1; from about 1:1 to about 11:1; from about 1:1 to about 10:1; from about 1:1 to about 9:1; from about 1:1 to about 8:1; from about 1:1 to about 7:1; from about 1:1 to about 6:1; from about 1:1 to about 5:1; from about 1:1 to about 4:1; from about 1:1 to about 3:1; or from about 1:1 to about 2:1.

In one embodiment, a compound of formula I and cefepime are administered in doses commonly employed when such agents are used individually for the treatment of a bacterial infection or disease.

In another embodiment, a compound of formula I and cefepime are administered in doses that are less than the doses commonly employed when such agents are used individually for the treatment of a bacterial infection or disease.

As used herein, cefepime includes a pharmaceutically acceptable salt thereof.

In one embodiment, the ratio of cefepime or pharmaceutically acceptable salt thereof to the compound of formula I or pharmaceutically acceptable salt of in vivo hydrolysable ester thereof is from about 1:1 to about 80:1; 1:1 to about 70:1; 1:1 to about 60:1; from about 1:1 to about 50:1; 1:1 to about 40:1; from about 1:1 to about 30:1; from about 1:1 to about 20:1; from about 1:1 to about 15:1; 1:1 to about 14:1; 1:1 to about 13:1; from about 1:1 to about 12:1; from about 1:1 to about 11:1; from about 1:1 to about 10:1; from about 1:1 to about 9:1; from about 1:1 to about 8:1; from about 1:1 to about 7:1; from about 1:1 to about 6:1; from about 1:1 to about 5:1; from about 1:1 to about 4:1; from about 1:1 to about 3:1; or from about 1:1 to about 2:1.

Cefepime can be administered to a patient at a dosage ranging from about 250 milligram (mg) to about 2g per administration. In one embodiment, the dosage of cefepime is about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1 g, about 1.1 g, about 1.2 g, about 1.25 g, about 1.3 g, about 1.4 g, about 1.5 g, about 1.6 g, about 1.7 g, about 1.75 g, about 1.8 g, or about 1.9 g. Cefepime can be administered at a time ranging from about every 8 hours to about every 48 hours. In another embodiment, cefepime is administered about every 12 hours, about every 16 hours, about every 20 hours, about every 24 hours, about every 28 hours, about every 32 hours, about every 36 hours, about every 40 hours, or every 44 hours. Cefepime can be administered for a duration ranging from about 7 days to about 10 days. In some embodiments, cefepime is administered over about 8 or 9 days.

When administered to a patient, a compound (e.g., a compound of formula I, cefepime, or a β-lactamase antibiotic) can be administered neat or as a component of a composition that comprises a physiologically acceptable carrier or vehicle. A composition can be prepared using a method comprising admixing the compound or a pharmaceutically acceptable salt of the compound and a physiologically acceptable carrier, excipient, or diluent. Admixing can be accomplished using methods well known for admixing a compound or a pharmaceutically acceptable salt of the compound and a physiologically acceptable carrier, excipient, or diluent.

Another embodiment provides for a composition comprising cefepime or a pharmaceutically acceptable salt thereof and a compound of formula I or a pharmaceutically acceptable salt or in vivo hydrolysable ester thereof. In another embodiment, the composition includes a compound of formula I or a pharmaceutically acceptable salt or in vivo hydrolysable ester thereof, and a composition of cefepime or a pharmaceutically acceptable salt thereof.

In another embodiment, there is provided a composition including a β-lactamase antibiotic or a pharmaceutically acceptable salt thereof and a compound of formula I or a pharmaceutically acceptable salt or in vivo hydrolysable ester thereof. In another embodiment, a composition is provided including a compound of formula I or a pharmaceutically acceptable salt or in vivo hydrolysable ester thereof, and a composition comprising a β-lactamase antibiotic or a pharmaceutically acceptable salt thereof.

The present compositions, comprising compounds or pharmaceutically acceptable salts of compounds can be administered orally. These compositions can also be administered by any other convenient route, for example, by continuous infusion or bolus injection, by absorption through epithelial or mucocutaneous linings (e.g., oral, rectal, vaginal, and intestinal mucosa, etc.) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in release of the compound or a pharmaceutically acceptable salt of the compound into the bloodstream. The mode of administration is left to the discretion of the practitioner.

In one embodiment, the compound or a pharmaceutically acceptable salt of the compound of formula I is administered orally.

In another embodiment, cefepime is administered orally.

In another embodiment, the β-lactamase antibiotic is administered orally.

In another embodiment, the compound or a pharmaceutically acceptable salt of the compound of formula I is administered intravenously.

In another embodiment, cefepime is administered intravenously.

In another embodiment, the β-lactamase antibiotic is administered intravenously.

In another embodiment, it may be desirable to administer the compound or a pharmaceutically acceptable salt of the compound of formula I locally. This can be achieved, for example, by local infusion during surgery, topical application, e.g., in conjunction with a wound dressing after surgery, by injection, by means of a catheter, by means of a suppository or edema, or by means of an implant, the implant being of a porous, non-porous, or gelatinous material, including membranes, such as, for example, sialastic membranes or fibers.

In some embodiments, it can be desirable to introduce the compound or a pharmaceutically acceptable salt of the compound of formula I into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as, an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compound or a pharmaceutically acceptable salt of the compound of formula I can be formulated as a suppository, with traditional binders and excipients such as, for example, triglycerides.

In another embodiment, the compound or a pharmaceutically acceptable salt of the compound of formula I can be delivered in a vesicle, in particular a liposome (see Langer, Science 1990, 249, 1527-1533 and Treat et al., Liposomes in the Therapy of Infectious Disease and Cancer 1989, 317-327 and 353-365).

In yet another embodiment, the compound or a pharmaceutically acceptable salt of the compound of formula I can be delivered in a controlled-release system or sustained-release system (see, e.g., Goodson, in Medical Applications of Controlled Release, vol. 2, 1984, 115-138). Other controlled or sustained-release systems discussed in the review by Langer, Science 1990, 249, 1527 1533 can be used. In one embodiment, a pump can be used (Langer, Science 1990, 249, 1527-1533; Sefton, CRC Crit. Ref. Biomed. Eng. 1987, 14, 201; Buchwald et al., Surgery 1980, 88, 507; and Saudek et al., N. Engl. J Med. 1989, 321, 574). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 1983 2, 61; Levy et al., Science 1935, 228, 190; During et al., Ann. Neural. 1989, 25, 351; and Howard et al., J. Neurosurg. 1989, 71, 105).

In yet another embodiment, a controlled- or sustained-release system can be placed in proximity of a target of the compound or a pharmaceutically acceptable salt of the compound of formula I, thus requiring only a fraction of the systemic dose.

The present compositions can optionally comprise a suitable amount of a physiologically acceptable excipient.

Such physiologically acceptable excipients can be liquids, such as, for example, water and oils, including those of petroleum, animal, vegetable, or synthetic origin, such as, for example, peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to a patient. The physiologically acceptable excipient should be stable under the conditions of manufacture and storage and should be preserved against the contaminating action of microorganisms. Water is a particularly useful excipient when the compound or a pharmaceutically acceptable salt of the compound is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers may be used in preparing solutions, suspensions, emulsions, syrups, and elixirs. The compound or pharmaceutically acceptable salt of the compound of formula I can be dissolved or suspended in a pharmaceutically acceptable liquid carrier such as, for example, water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as, for example, ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule. Other examples of suitable physiologically acceptable excipients are described in Remington's Pharmaceutical Sciences 1447 1676 (Alfonso R. Gennaro, ed., 19th ed. 1995).

In one embodiment, the compound or a pharmaceutically acceptable salt of the compound of formula I is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs for example. Orally administered compositions can contain one or more agents, for example, sweetening agents such as, for example, fructose, aspartame or saccharin; flavoring agents such as, for example, peppermint, oil of wintergreen, or cherry; coloring agents; and preserving agents, to provide a pharmaceutically palatable preparation. In powders, the carrier can be a finely divided solid, which is an admixture with the finely divided compound or pharmaceutically acceptable salt of the compound. In tablets, the compound or pharmaceutically acceptable salt of the compound is mixed with a carrier having the necessary compression properties in suitable proportions and compacted in the shape and size desired. The powders and tablets can contain up to about 99% of the compound or pharmaceutically acceptable salt of the compound.

Capsules may contain mixtures of the compounds or pharmaceutically acceptable salts of the compounds with inert fillers and/or diluents such as, for example, pharmaceutically acceptable starches (e.g., corn, potato, or tapioca starch), sugars, artificial sweetening agents, powdered celluloses (such as, for example, crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as, for example, glycerol monostearate or glycerol stearate can also be used. Oral compositions can include standard excipients such as, for example, mannitol, lactose, starch, magnesium stearate, sodium saccharin, cellulose, and magnesium carbonate. In one embodiment the excipients are of pharmaceutical grade.

In another embodiment, the compound or a pharmaceutically acceptable salt of the compound of formula I can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as, for example, lignocaine to lessen pain at the site of the injection. Generally, the ingredients are supplied either separately or mixed together in unit dosage form, for example, as a dry lyophilized powder or water-free concentrate in a hermetically sealed container such as, for example, an ampoule or sachette indicating the quantity of active agent. Where the compound or a pharmaceutically acceptable salt of the compound of formula I is to be administered by infusion, it can be dispensed, for example, with an infusion bottle containing sterile pharmaceutical grade water or saline. Where the compound or a pharmaceutically acceptable salt of the compound is administered by injection, an ampule of sterile water for injection or saline can be provided so that the ingredients can be mixed prior to administration.

In another embodiment, the compound or pharmaceutically acceptable salt of the compound of formula I can be administered transdermally through the use of a transdermal patch. Transdermal administrations include administrations across the surface of the body and the inner linings of the bodily passages including epithelial and mucosal tissues. Such administrations can be carried out using the present compounds or pharmaceutically acceptable salts of the compounds, in lotions, creams, foams, patches, suspensions, solutions, and suppositories (e.g., rectal or vaginal).

Transdermal administration can be accomplished through the use of a transdermal patch containing the compound or pharmaceutically acceptable salt of the compound and a carrier that is inert to the compound or pharmaceutically acceptable salt of the compound, is non-toxic to the skin, and allows delivery of the agent for systemic absorption into the blood stream via the skin. The carrier may take any number of forms such as, for example, creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the compound or pharmaceutically acceptable salt of the compound into the blood stream, such as, for example, a semi-permeable membrane covering a reservoir containing the compound or pharmaceutically acceptable salt of the compound with or without a carrier, or a matrix containing the active ingredient.

The compounds or pharmaceutically acceptable salts of the compounds of formula I may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as, for example, polyethylene glycols of various molecular weights, may also be used.

The compound or a pharmaceutically acceptable salt of the compound of formula I can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the compounds of the present teachings. A single unit dosage form suitable for oral administration such as, for example, tablets, capsules, gelcaps, and caplets that are adapted for controlled- or sustained-release.

In one embodiment a controlled- or sustained-release composition comprises a minimal amount of the compound or a pharmaceutically acceptable salt of the compound of formula Ito treat or prevent a bacterial infection or disease in a minimal amount of time. Advantages of controlled- or sustained-release compositions include extended activity of the drug, reduced dosage frequency, and increased compliance by the patient being treated. In addition, controlled or sustained release compositions can favorably affect the time of onset of action or other characteristics, such as, for example, blood levels of the compound or a pharmaceutically acceptable salt of the compound, and can thus reduce the occurrence of adverse side effects.

Controlled- or sustained-release compositions can initially release an amount of the compound or a pharmaceutically acceptable salt of the compound of formula I that promptly produces the desired therapeutic or prophylactic effect, and gradually and continually release other amounts of the compound or a pharmaceutically acceptable salt of the compound to maintain this level of therapeutic or prophylactic effect over an extended period of time. To maintain a constant level of the compound or a pharmaceutically acceptable salt of the compound of formula I in the body, the compound or a pharmaceutically acceptable salt of the compound of formula I can be released from the dosage form at a rate that will replace the amount of the compound or a pharmaceutically acceptable salt of the compound being metabolized and excreted from the body. Various conditions, including but not limited to, changes in pH, changes in temperature, concentration or availability of enzymes, concentration or availability of water, or other physiological conditions or compounds can stimulate controlled- or sustained-release of an active ingredient.

In some embodiments, the present teachings are directed to prodrugs of the compounds or pharmaceutically acceptable salts of compounds of formula I. Various forms of prodrugs are known in the art, for example as discussed in Bundgaard (ed.), Design of Prodrugs, Elsevier 1985; Widder et al. (ed.), Methods in Enzymology, vol. 4, Academic Press 1985; Kgrogsgaard-Larsen et al. (ed.); “Design and Application of Prodrugs”, Textbook of Drug Design and Development, 1991, Chapter 5, 113-191; Bundgaard et al., Journal of Drug Delivery Reviews, 1992, 8, 1-38; Bundgaard et al., J. Pharmaceutical Sciences, 1988, 77, 285 et seq.; and Higuchi and Stella (eds.), Prodrugs as Novel Drug Delivery Systems, American Chemical Society (1975).

The amount of the compound or a pharmaceutically acceptable salt of the compound of formula I is an amount that is effective for treating a bacterial infection or disease. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound or a pharmaceutically acceptable salt of the compound is administered, the effective dosage amounts correspond to the total amount administered.

The amount of the compound or a pharmaceutically acceptable salt of the compound of formula I that is effective for treating a bacterial infection or disease will typically range from about 0.001 mg/kg to about 250 mg/kg of body weight per day, or from about 1 mg/kg to about 250 mg/kg body weight per day, or from about 1 mg/kg to about 50 mg/kg body weight per day, or from about 1 mg/kg to about 20 mg/kg of body weight per day.

In some embodiments, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may be given in a single dose or in two or more divided doses.

The compound or a pharmaceutically acceptable salt of the compound of formula I can be assayed in vitro or in vivo for the desired therapeutic or prophylactic activity prior to use in humans. Animal model systems can be used to demonstrate safety and efficacy.

### Examples

The bicyclic and the tricyclic aldehydes required to prepare the final compounds were prepared by following the procedures as outlined in US patents U.S. Pat. No. 7,112,582 (2006) and U.S. Pat. No. 0,074,064 (2006) each of which are incorporated herein by reference in there entirety.

### Example 1

### (Scheme 15)

### (5R), (6E)-6-(6,7-dihydro-5H-pyrrolo[1,2-a]imidazol-2-ylmethylene)-7-oxo-4-thia-1-aza-bicyclo[3.2.0]hept-2-ene-2-carboxylic acid

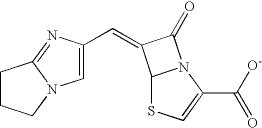

Step 1: (3R,4R)-4-allyI-3-bromoazetidin-2-one

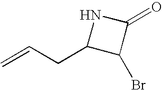

(3R,4R)-4-allyl-3-bromoazetidin-2-one was prepared by the procedure as outlined in S. Coulton et al. (J.C.S.Parkin Trans., 2699 (1991)).

Step 2: {(2R,3R)-2-allyl-3-bromo-4-oxo-azetidin-1-yl)-(triphenyl-5-phosphanylidene)}-acetic acid 4-nitro-benzyl ester

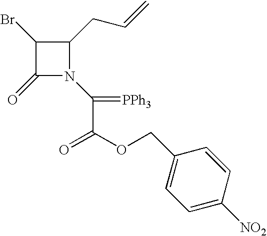

A solution of p-nitrobenzyl glyoxylate monohydrate (22.6 grams (g)) in benzene (1500 milliliter (ml)) was heated to reflux with the provision to remove water (Dean-Stark) for 1.5 hours. After the mixture was cooled to room temperature and a solution of (3R,4R)-4-allyl-3-bromoazetidin-2-one (18.6g) in benzene (200 ml) containing Et₃N (1.4 ml) was added and the mixture was stirred for 19 hours before being evaporated at reduced pressure to yield a crude material as an amorphous solid.

The crude material was dissolved in dry tetrahydrofuran (THF) (740 ml) and the solution was cooled to minus 20 degrees Celsius (° C.) under nitrogen. To the stirred solution was added 2,6-Lutidine (17.2 ml) and SOCl₂ (11 ml) and the mixture was stirred for 15 minutes. The resulting suspension was filtered and the filtrate was evaporated at reduced pressure to yield a crude material as an oil.

The crude material was dissolved in 1,4-dioxane (200 ml) and the solution was stirred with Ph₃P (77.7 g) for 20 minutes. The mixture was heated to 50° C. under nitrogen for 2 hours with 2,6-Lutidine (13.8 ml). The solution was then diluted with AcOEt and washed with water, 5% aqueous citric acid and brine. The organic layers was dried (MgSO₄), filtered and evaporated under reduced pressure. The residue was applied to silica gel column chromatography and eluted with AcOEt-Hex with a gradient (3:4-4:3) to obtain the title compound as a yellow amorphous solid (35.9 g, 57%). 1H NMR (CDCl₃) δ 2.6-2.8 (m, 2H), 4.2 (m, 1H), 4.87 (d, 1H, J=4.4 Hz), 5.1 (m, 2H), 5.27 (d, 2H, J is 13.6 Hz), 5.8-5.9 (m, 1H), 7.4-7.7 (m, 15H).

Step 3: (5R, 6S)-6-Bromo-7-oxo-1-aza-bicyclo[3.2.0]hept-2-ene-2-carboxylic acid 4-nitrobenzyl ester

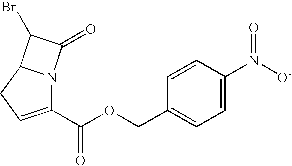

Phosphorane {(2R,3R)-2-Allyl -3-bromo-4-oxo-azetidin-1-yl)-(triphenyl-5-phosphanylidene)}-acetic acid 4-nitro-benzyl ester (3.77 g) was dissolved in AcOEt (65.6 ml) and the solution was cooled to 0° C. TFA (21.8 ml) was added to the stirred solution. After 15 minutes at 0° C. the solution was cooled to minus 70° C. and a stream of ozonolyzed oxygen was passed through the solution for 2 minutes. In the case of longer than 2 minutes over-oxidation occurred to reduce the yield. The excess of ozone was blown off with nitrogen for 30 minutes and a solution of Ph₃P (1.53 g) in AcOEt (11.7 ml) was added. The solution was allowed to reach room temperature and was then neutralized with aqueous NaHCO₃. The organic layers was washed with brine, dried (MgSO₄), and evaporated under reduced pressure to yield a crude solid. The residue was triturated with AcOEt (3 ml) and THF (3 ml) and filtered to give the title compound as a colorless crystal (1.1 g, 51%). 1H NMR (CDCI₃) δ2.85-2.93 (m, 1H), 3.05-3.14 (m, 1H), 4.42-4.47 (m, 1H), 4.76 (d, 1H, J=2.9 Hz), 5.31 (d, 1H, J=13.5 Hz), 5.45 (d, 1H, J=13.5 Hz), 6.58 (t, 1H, J=2.9 Hz), 7.62 (d, 1H, J=8.9 Hz), 8.24 (d, 1H, J=8.9 Hz).

Step 4: (5R), (6E)-6-(2,3-dihydro-imidazo[2,1-b]thiazol-5-ylmethylene)-7-oxo-1-aza-bicyclo[3.2.0]hept-2-ene-2-carboxylic acid, sodium salt (Compound 1)

7H-Imidazo[2,1-Nthiazole-2-carbaldehyde (277mg) was added to the dry acetonitrile (12.8 mL) solution of anhydrous MgBr₂ (479 mg) under a nitrogen atmosphere at room temperature. The dry THF solution (12.8 ml) of (5R, 6S)-6-Bromo-7-oxo-1-aza-bicyclo[3.2.0]hept-2-ene-2-carboxylic acid 4-nitrobenzyl ester (551 mg) was added to the mixture, cooled to minus 20° C., and Et₃N (0.71 ml) was added in one portion. The reaction vessel was covered with foil to exclude light. The reaction mixture was stirred for 1 hours at minus 20° C. and stirred for additional 1.5 hours at 5° C. then treated with acetic anhydride (0.28 mL) and DMAP (37 mg) in one portion. The reaction mixture was warmed to 0° C. and stirred for 19 hours at 0° C. The mixture was diluted with ethyl acetate and washed with 5% citric acid aqueous solution, saturated sodium hydrogen carbonate, water and brine. The organic layer was dried (MgSO₄) and filtered. The filtrate was concentrated under reduced pressure.

The residue was dissolved in THF (12.2 ml) and acetonitrile (5.7 ml). Freshly activated Zn dust (3.3 g) and 0.5 Molar (M) phosphate buffer (pH 6.5, 17.9 ml) were added to the mixture. The reaction vessel was covered with foil to exclude light. The reaction mixture was vigorously stirred for 1.5 hours at 35° C., cooled to 5° C. and 1 M NaOH was added to adjust pH to 7.5. The reaction mixture was diluted with ethyl acetate and filtered through a pad of Celite. The pad was washed with water and the aqueous layer was separated. The aqueous layer was cooled to 5° C. and stored overnight. The mixture was concentrated under high vacuum at 35° C. The concentrate was applied to Diaion HP-21 (50 ml, Mitsubishi Kasei Co. Ltd.) resin column chromatography. After adsorbing, the column was eluted with H₂O—MeCN (1:0-9/1). The combined fractions were concentrated under high vacuum at 35° C. and lyophilized to give the title compound as a yellow amorphous solid (97 milligram (mg), 21%, pH 7.5). 1H NMR (D₂O) δ2.38-2.46 (m, 1H), 2.71-2.79 (m, 1H), 3.71 (t, 2H, J=7.3 Hz), 4.01-4.11 (m, 2H), 4.76-4.80 (m, 1H), 6.06 (t, 1H, J=2.6 Hz), 6.64 (s, 1H), 7.27 (s, 1H).

### Example 2

### IC₅₀ Determination for the Penem Inhibitor

β-Lactamase inhibitory activity of the penem inhibitors was determined spectrophotometrically as described by Bush et al., [Bush, K., Macalintal, C., Rasmussen, B. A., Lee, V. and Yang, Y. Antimicrobial Agents and Chemotherapy 1993, 37, 851]. Homogeneously purified class A, β-lactamases TEM-1 from E. coli and Imi-1 from Enterobacter cloacae, class B enzyme CcrA from Bacteroides fragilis and class C enzyme AmpC from Enterobacter cloaca were employed in the assay. The enzyme concentrations for TEM-1, Imi-1, CcrA and AmpC were 4.3, 7.1, 1.2 and 2.1 nanomolar (nM), respectively. A wide range of inhibitor concentrations were prepared in 50 millimolar (mM) PO₄, pH 7.0 to include the possible IC₅₀ values. The substrate used to initiate the enzyme reaction was nitrocefin at 50 μg/ml in the same buffer as the inhibitor. Initially the enzyme and inhibitor (20 μl each) were preincubated for 10 minutes at 25° C. prior to the addition of 160 μl volume of nitrocefin. Initial rates of hydrolysis were monitored for 5 minutes at 495 nm using a Molecular Devices Spectra Max 250 with kinetic protocol of SoftMax Program. Readings from the Spectra Max 250 were exported and transferred to Microsoft Excel. The percent of inhibition of each inhibitor concentration was calculated based on the control enzyme activity. The inhibitor concentration that caused a 50% reduction in the enzymatic activity (1050) was determined graphically.

| Example # | TEM-1 (nM) | AmpC (nM) |
|---|---|---|
| Example 1 | 80 | 120 |

Antimicrobial Susceptibility Testing.

The in vitro activities of the antibiotics were determined by the microbroth dilution method as recommended by the National Committee for Clinical Laboratory Standards (NCCLS 2000). Methods for Dilution Antimicrobial Susceptibility Tests for Bacteria That Grow Aerobically; Approved Standards: M7-A5, vol. 19. National Committee for Clinical Laboratory Standards, Villanova, Pa.). Mueller-Hinton II broth (MHBII)(BBL Cockeysville, Md.), was used for the testing procedure. Microtiter plates containing 50 μl per well of two-fold serial dilutions of piperacillin combined with a constant amount (4 ug/ml) of a β-lactamase inhibitor were inoculated with 50 μl of inoculum to yield the appropriate density (10⁵ CFU/ml) in 100×l final volume. The plates were incubated for 18 to 22 hours at 35° C. in ambient air. The minimal inhibitory concentration (MIC) for all isolates was defined as the lowest concentration of antimicrobial agent that completely inhibits the growth of the organism as detected by the unaided eye. The MIC data obtained by the above said procedure are given below.

Experimental Protocol

Mice are challenged by injecting 0.5 ml intraperitoneally or 0.05 ml intranasally of a predetermined bacterial inoculum suspended in broth, saline or hog gastric mucin (supplemented with dried bovine hemoglobin for N. gonorrhoeae). The bacterial inoculum is equivalent to 10-100 LD₅₀s of the specific infecting strain and will result in death of the non-treated control animals within 7 days: “Bacterial Virulence in Mice”. Antibacterial doses (dose concentration prepared by two fold serial dilutions of the antibiotic) are dissolved or suspended in 0.2% aqueous agar or methocel, phosphate buffered saline or an adjuvant are administered orally, subcutaneously or intravenously in the following manner:

a) Orally or subcutaneously: Dose volume of 0.5 ml administered ½ hour after infection. A second dose may be administered 3 hours after infection for treatment of infections with more virulent organisms.

b) Intravenously: Dose volume of 0.2 ml, administered ½ hour after infection. For the treatment of infections with more virulent organisms, more doses, up to 48 hours may be administered. (Intravenous dosing will not exceed 3 doses/24 hour period.)

c) Oral pretreatment : Under special circumstances, the pH of the stomach needs to be adjusted in order to increase the gastric stability of the antibiotic. For this purpose, 0.5 ml of phosphate buffered saline (pH7.8, 0.06M) (or specific approved adjuvant) is administered orally ½ hour after infection, followed 5 minutes later by 0.5 ml of antibiotic (also orally) contained in phosphate buffered saline (pH7.8, 0.06M).

Animal Species

A detailed explanation as to the number of animals needed for the determination of in vivo efficacy follows:

a) Antibiotics are tested at 5 different dose levels with 5 mice per dose level at each of three routes of administration (oral, subcutaneous and intravenous). Initially the three routes of administration should be investigated so as to determine if the drug is orally absorbed and/or which is the most effective route. This would require 25 mice/route with 3 routes/antibiotic or 75 mice per compound tested. One to two antibiotics will be tested per experiment (75-150 mice)

b) The effectiveness of the new compound is compared to that of a standard, or antibiotic of known effectiveness. Known or previously tested antibiotics are tested at 5 dose levels with 5 mice per dose level by a single route of administration, for a total of 25 mice/antibiotic. Usually 3-6 antibiotics will be tested per experiment. (75-150 mice).

c) Untreated controls—In each of the above tests, untreated animals are infected with 3 different concentrations of bacterial inoculum with 10 mice per concentration (30 mice total in each and every test). These untreated controls are used to determine and maintain the infection level between 10-100 LD50's as required for test-to-test comparison and validity.

Determination of Protective Effects of Antibacterial Agents:

The protective effects of the antibacterial agent(s) are measured by the survival of the infected untreated as compared to the treated animals. For this determination, animals are observed for 7 days after treatment. A census of survivors is taken twice daily and at that time dead as well as moribund animals are removed. The 7 day survival ratio from three separate tests are pooled for estimation of median effective dose (ED50) by computerized program for probit analysis (Cleeland, R. and E. Squires. 1991. Evaluation of New Antimicrobials in Vitro and in Experimental Animal Infections. In Antibiotics in Laboratory Medicine, 3rd. ed., edited by Victor Lorian. Williams and Wilkins Baltimore, Md. pp. 752-783).

The test is performed three times on separate days to provide a statistically valid number of animals and to minimize variation in test results on a day-to-day and test-to-test basis.

Variations, modifications, and other implementations of what is described herein will occur to those of ordinary skill in the art without departing from the spirit and the essential characteristics of the present teachings. Accordingly, the present teachings are intended to include all such modifications and implementations, and their equivalence.

Each reference cited in the present application, including books, patents, published applications, journal articles and other publications, is incorporated herein by reference in its entirety.

1. A compound of Formula I: 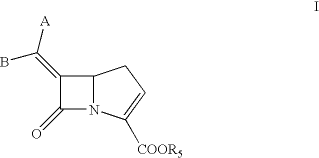
wherein:
one of A and B denotes hydrogen and the other an 8- to 14-membered fused bicyclic or tricyclic heteroaryl group optionally substituted with nitro, C₆₋₁₄aryl, -heteroaryl, alkoxycarbonyl-, C₁₋₆alkoxy, -alkoxyalkyl, alkyl-O—C₂₋₄alkyl-O—, -cyano, -halogen, -hydroxy, —N—R₆R₇, -trifluoromethyl, -trifluoromethoxy, arylalkyl, alkylaryl, R₆R₇N-alkyl-, HO—C₁₋₆alkyl-, alkoxyalkyl-, C₁₋₆alkyl-S—, —SO₂N—R₆R₇, —SO₂NHR₆, —CO₂H, CONR₆R₇, C₆₋₁₄aryl-O—, heteroaryl-O—, —S(═O)ₛ-aryl where s is 0-2, -alkyl-O—-alkyl-NR₆R₇, -alkyl-aryl-O-alkylN—R₆R₇, C₁₋₆alkyl, C₂₋₈alkenyl, C₂₋₆alkynyl, C₃₋₇cycloalkyl, alkoxy-alkyl-O—, R₆R₇N—(C₁₋₆alkyl), and —S(═O)ₛ-heteroaryl where s is 0-2;
R₅ is H, C₁₋₆alkyl, C₅₋₆cycloalkyl, or CHR₃OCOC₁₋₆alkyl;
R₃ is hydrogen, C₁₋₆alkyl, C₃₋₇cycloalkyl, C₆₋₁₄aryl, or 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, wherein aryl or heteroaryl can be optionally substituted with 1 or 2 of nitro, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl, C₁₋₆alkoxy, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, alkyl-S—, —SO₂N—R₆R₇, aryl-O—, heteroaryl-O—, —S((═O)ₛ-aryl where s is 0-2, -alkyl-O-alkyl-NR₆R₇, -alkyl-aryl-O-alkylN—R₆R₇, -alkyl-aryl-O—-alkylN—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; and
R₆ and R₇ are independently H, or a group selected from C₁₋₆alkyl, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, alkylaryl, arylalkyl, heteroarylalkyl, C₁₋₆alkylheteroaryl, said group being optionally substituted with nitro, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl-, C₁₋₆alkoxy, -alkoxyalkyl, alkyl-O—C₂₋₄alkyl-O-cyano, -halogen, -hydroxy, —NR₆R₇, —COOH, —COO-alkyl, -trifluoromethyl, -trifluoromethoxy, arylalkyl, alkylaryl, R₆R₇N-alkyl-, HO—C₁₋₆alkyl, alkoxyalkyl-, C₁₋₅alkyl-S—, —SO₂NHR₆, —CO₂H, CONR₆R₇, C₆₋₁₄aryl-O—, heteroaryl-O—, —S(═O)ₛ-aryl, wherein s is 0-2, -alkyl-O-alkyl-NR₆R₇, -alkyl-aryl-O-alkyl-N—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl, where s is 0-2; or R₆ and R₇ can together with the nitrogen to which they are attached form a 3 to 7 membered saturated ring system optionally having in addition to the nitrogen, one or two heteroatoms selected from N—R₁, O, and S(═O)ₙ, where n is 0-2;
or a pharmaceutically acceptable salt thereof.

2. A compound according to claim 1, wherein A or B is a bicyclic heteroaryl group of the formula: 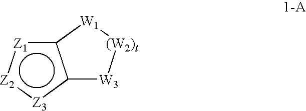
wherein:
one of Z₁, Z₂ and Z₃ is a carbon bonded to the remainder of the molecule and the other two are independently selected from CR₂, N, O, S, and N—R₁;
W₁, W₂ and W₃ are each independently selected from CR₄R₄, S, SO, SO₂, O, N—R₁, and C(═O), with the proviso that whenever W₂ is S or O it is not adjacent to a W₁, W₂ or W₃ that is also S or O;
t is 1-4;
R₁ is H, or is a group selected from C₁₋₆alkyl, C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₃₋₇cycloalkyl, C₂₋₈alkenyl, C₂₋₆alkynyl, C₁₋₆perfluoroalkyl, —S(═O)₂C₁₋₆alkyl or C₆₋₁₄aryl, —C(═O)heteroaryl, —C(═O)aryl, —C(═O)(C₁₋₆alkyl), —C(═O)C₃₋₆cycloalkyl, —C(═O) mono or bicyclic saturated heterocyclyl, C₁₋₆alkylaryl, (C₁₋₆alkyl)heteroaryl, aryl(C₁₋₆alkyl), heteroaryl(C₁₋₆alkyl), C₁₋₆alkyl mono or bicyclic saturated heterocyclyl, arylalkenyl of 8 to 16 carbon atoms, arylalkyloxyalkyl, (C₁₋₆alkyl )O(C₁₋₆alkyl)-aryl, (C₁₋₆alkyl )O(C₁₋₆alkyl)heteroaryl, aryloxy heteroaryloxyalkyl, aryloxyaryl, aryloxyheteroaryl, alkylaryloxyaryl, alkylaryloxyheteroaryl, alkylaryloxyalkylamines, saturated heterocyclyl carbonyl, aryloxy carbonyl, heteroaryloxy carbonyl, —CONR₆R₇, and —SO₂NR₆R₇, said group being optionally substituted with nitro, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, alkoxycarbonyl, C₁₋₆alkoxy, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, alkyl-S—, —SO₂N—R₆R₇, aryl-O—, heteroaryl-O—, —S((═O)ₛ-aryl where s is 0-2, -alkyl-O-alkyl-NR₆R₇, -alkyl-aryl-O-alkylN—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; provided the atom of R₁ which bonds to N is not a carbon that is double or triple bonded to another carbon;
R₂ is absent, hydrogen, halogen, cyano, N—R₆R₇, hydroxy; COOR₆, C₁₋₆alkylamino (C₁₋₆alkoxy), alkylenedioxy, C₁₋₆perfluoroalkyl, CONR₆R₇, guanidino or cyclic guanidino, SO₂NR₆R₇, C₁₋₆alkyl, C₂₋₈alkenyl having 1 to 2 double bonds, C₂₋₆alkynyl having 1 to 2 triple bonds; C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxy, alkylaryloxy alkylamines, aryloxy, heteroaryloxy, alkenyloxy, alkynyloxy, aryloxyalkyl amine, C₁₋₆perfluoroalkyl, S(═O)_{q}—C₁₋₆alkyl, S(═O)_{q}— where q is 0-2, alkylaryl, arylalkyl, C₁₋₆alkylheteroaryl, heteroaryl-C₁₋₆alkyl, C₁₋₆alkyl mono or bicyclic saturated heterocyclyl having 1-3 heteroatoms independently selected from O, N or S, arylalkenyl of 8 to 16 carbon atoms, arylalkyloxyalkyl, aryloxyalkyl, heteroaryloxyalkyl, aryloxyaryl, aryloxyheteroaryl, heteroaryloxyaryl, alkyl aryloxyaryl, alkylaryloxyheteroaryl, aryloxyalkyl, heteroaryloxyalkyl, alkylaryloxyalkylamines, C₃₋₇cycloalkyl, saturated or partially saturated heterocyclyl, said group being optionally substituted with nitro, C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl, C₁₋₆alkoxy, -alkoxyalkyl, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, C₁₋₆alkyl-S—, —SO₂N—R₆R₇, C₆₋₁₄aryl-O—, heteroaryl-O—, —S((═O)ₛ-aryl where s is 0-2, -alkyl-O-alkyl-NR₆R₇, -alkyl-aryl-O—- alkylN—R₆R₇, -alkyl-aryl-O-alkylN—R₅R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; provided that the atom of R₂ that bonds to N is not a carbon that is double or triple bonded to another carbon;
each R₄ is independently H or C₁₋₆alkyl optionally substituted with nitro, C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl, C₁₋₆alkoxy, -alkoxyalkyl, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, C₁₋₆alkyl-S—, —SO₂N—R₆R₇, C₆₋₁₄aryl-O-, heteroaryl-O—, —S((═O)₅-aryl where s is 0-2, -alkyl-O-alkyl-NR₆R₇, -alkyl-aryl-O-alkylN—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; or any one of R₄ can be OH, C₁₋₆alkoxy, —S—C₁₋₆alkyl, COOR₆, —NR₆R₇, —CONR₆R₇; or R₄R₄ may together be (═O); or R₄R₄together with the carbon to which they are attached may form a spiro ring of five to eight members optionally having 1-3 heteroatoms selected from N, O, S(═O), where n is 0-2, and N—R₁; and
R₆ and R₇ are as defined in claim 1.

3. A compound according to claim 1, wherein A or B is a bicyclic heteroaryl group of the formula 1-B: 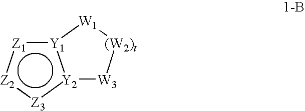
wherein:
Y₁ and Y₂ are independently C or N;
t is 1-4; and
Z₁, Z₂, Z₃, W₁, W₂, W₃, R₁, R₂, R₄, R₆, and R₇ are as defined in claim 2.

4. A compound according to claim 3, wherein Z₁, Z₂, W₁, and W₂ are CR₂, Z₃ and Y₁ are N, Y₂, W₃ is S; and
R₁, R₂, R₄, R₆, and R₇ are as defined in claim 2.

5. A compound according to claim 4 wherein R₂ is H.

6. A compound according to claim 1, wherein A or B is a bicyclic heteroaryl group of the formula 1-C 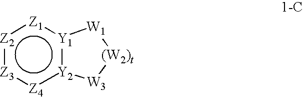
wherein:
one of Z₁, Z₂, Z₃ and Z₄ is a carbon bonded to the remainder of the molecule and the other three are independently CR₂ or N;
t is 1-4; and
W₁, W₂, W₃, Y₁, Y₂, and R₂ are as defined in claim 3.

7. A compound according to claim 2 wherein one of Z₁, Z₂, or Z₃ is CR₂.

8. A compound according to claim 2, wherein at least one of Z₂ or Z₃ is N, O, or S.

9. A compound according to claim 2, wherein at least one of W₁, W₂, and W₃ is CR₄R₄.

10. A compound according to claim 2, wherein W₁, W₂, and W₃ are all independently CR₄R₄.

11. A compound according to claim 2, wherein t is 1-3.

12. A compound according to claim 3, wherein t is 3.

13. A compound according to claim 3, wherein at least two of Z₁, Z₂, Z₃, Y₁, and Y₂ are N.

14. A compound according to claim 3, wherein three of Z₁, Z₂, Z₃, Y₁, and Y₂ are N.

15. A compound according to claim 3, wherein two of W₁, W₂, and W₃ are independently CR₄R₄.

16. A compound according to claim 15, wherein each R₄ is H.

17. A compound according to claim 3, wherein one of Y₁ and Y₂ is CR₂ and the other is N.

18. A compound according to claim 3, wherein two of Z₁, Z₂, and Z₃ are independently CR₂.

19. A compound according to claim 6, wherein at least three of Z₁, Z₂, Z₃, and Z₄ are independently CR₂.

20. A compound according to claim 6, wherein at least one of Z₁, Z₂, Z₃, and Z₄ is N.

21. A compound according to claim 20, wherein Z₁ is N.

22. A compound according to claim 6, wherein at least two of Z₁, Z₂, Z₃, and Z₄ are N.

23. A compound according to claim 6 wherein at least three of Z₁, Z₂, Z₃, and Z₄ are N.

24. A compound according to claim 1, wherein A or B is a tricyclic heteroaryl group of the formula 2-A or 2-B 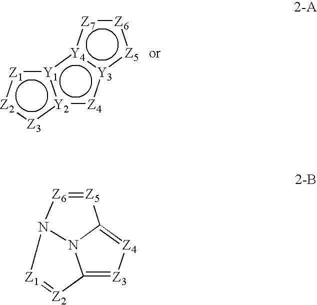
wherein:
one of Z₁, Z₂, Z₃, Z₄, Z₅, Z₆, and Z₇ is a carbon bonded to the remainder of the molecule and the other Z₁, Z₂, Z₃, Z₄, Z₅, Z₆ and Z₇ are independently selected from CR₂, N, O, S and N—R₁, with the proviso that in formula 2-B, Z₁, Z₂, Z₃, Z₄, Z₅ and Z₆ are selected from CR₂ and N;
Y₁, Y₂, Y₃ and Y₄ are each independently C or N;
R₁ is H, —CONR₆R₇, —SO₂NR₆R₇, or is a group selected from C₁₋₆ealkyl, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₃₋₇cycloalkyl, C₂₋₈alkenyl, C₂₋₆alkynyl, C₁₋₆perfluoroalkyl, —S(═O)₂C₁₋₆alkyl or C₆₋₁₄aryl, —C(═O) heteroaryl, —C(═O)aryl, —C(═O)(C₁₋₆alkyl), —C(═O)C₃₋₆cycloalkyl, —C(═O) mono or bicyclic saturated heterocyclyl, C₁₋₆alkylaryl, (C₁₋₆alkyl)heteroaryl, aryl(C₁₋₆alkyl), heteroaryl(C₁₋₆alkyl), C₁₋₆alkyl mono or bicyclic saturated heterocyclyl, arylalkenyl of 8 to 16 carbon atoms, arylalkyloxyalkyl, (C₁₋₆alkyl )O(C₁₋₆alkyl)-aryl, (C₁₋₆alkyl )O(C₁₋₆alkyl)heteroaryl, aryloxyalkyl, heteroaryloxyalkyl, aryloxyaryl, aryloxyheteroaryl, alkylaryloxyaryl, alkylaryloxyheteroaryl, alkylaryloxyalkylamines, C₁₋₆alkoxycarbonyl, aryloxy carbonyl, heteroaryloxy carbonyl, said group being optionally substituted with nitro, -aryl, heteroaryl, C₁₋₆alkoxycarbonyl, C₁₋₆alkoxy, -alkoxyalkyl, alkyl-O—C₂₋₄alkyl-O—, -cyano, -halogen, -hydroxy, —NR₆R₇, -trifluoro methyl, -trifluoromethoxy, arylalkyl, alkylaryl, R₆R₇N-alkyl-, HO—C₁₋₆alkyl-, alkoxyalkyl-, alkyl-S—, —SO₂N—R₆R₇, aryl-O—, heteroaryl-O—, —S((═O)ₛ-aryl where s is 0-2, -alkyl-O-alkyl-NR₆R₇, -alkyl-aryl-O-alkylN—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; provided the atom of R₁ which bonds to N is not a carbon that is double or triple bonded to another carbon.
R₂ is absent, hydrogen, halogen, cyano, N—R₆R₇, hydroxy; COOR₆, C₁₋₆alkylamino (C₁₋₆alkoxy), alkylenedioxy, C₁₋₆perfluoroalkyl, CONR₆R₇, guanidino or cyclic guanidino, SO₂NR₆R₇, C₁₋₆alkyl, C₂₋₈alkenyl having 1 to 2 double bonds, C₂₋₆alkynyl having 1 to 2 triple bonds; C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxy, alkylaryloxy alkylamines, aryloxy, heteroaryloxy, alkenyloxy, alkynyloxy, aryloxyalkyl amine, C₁₋₆perfluoroalkyl, S(═O)_{q}—C₁₋₆alkyl, S(═O)_{q} where q is 0-2, alkylaryl, arylalkyl, C₁₋₆alkylheteroaryl, heteroaryl-C₁₋₆alkyl, C₁₋₆alkyl mono or bicyclic saturated heterocyclyl having 1-3 heteroatoms independently selected from O, N or S, arylalkenyl of 8 to 16 carbon atoms, arylalkyloxyalkyl, aryloxyalkyl, heteroaryloxyalkyl, aryloxyaryl, aryloxyheteroaryl, heteroaryloxyaryl, alkyl aryloxyaryl, alkylaryloxyhethoaryl, aryloxyalkyl, heteroaryloxyalkyl, alkylaryloxyalkylamines, C₃₋₇cycloalkyl, saturated or partially saturated heterocyclyl, said group being optionally substituted with nitro, C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S,C₁₋₆alkoxycarbonyl, C₁₋₆alkoxy, -alkoxyalkyl, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, C₁₋₆alkyl-S—, —SO₂N—R₆R₇, C₆₋₁₄aryl-O—, heteroaryl-O—, —S((═O)ₛ-aryl where s is 0-2, -alkyl-O-alkyl-NR₆R₇, -alkyl-aryl-O-alkylN—R₆R₇, -alkyl-aryl-O-alkylN—R₅R₇, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; provided that the atom of R₂ that bonds to N is not a carbon that is double or triple bonded to another carbon;
R₆ and R₇ are independently H, or a group selected from C₁₋₆alkyl, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, alkylaryl, arylalkyl, heteroarylalkyl, C₁₋₆alkylheteroaryl, said group being optionally substituted with nitro, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl-, C₁₋₆alkoxy, -alkoxyalkyl, alkyl-O—C₂₋₄alkyl-O-cyano, -halogen, -hydroxy, —NR₆R₇, —COOH, —COO-alkyl, -trifluoromethyl, -trifluoromethoxy, arylalkyl, alkylaryl, R₆R₇N-alkyl-, HO—C₁₋₆alkyl, alkoxyalkyl-, C₁₋₆alkyl-S-, —SO₂NHR₆, —CO₂H, CONR₆R₇, C₆₋₁₄aryl-O—, heteroaryl-O—, —S(═O)ₛ-aryl, wherein s is 0-2, -alkyl-O-alkyl-NR₆R₇, C₁₋₆alkyl, C₂₋₆alkenyl, C₂₋₆alkynyl, C₃₋₇cycloalkyl, alkoxy-alkyl-O—, R₆R₇N-alkyl-, and —S(═O)ₛ-heteroaryl, wherein s is 0-2, or R₆ and R₇ can together with the nitrogen to which they are attached form a 3 to 7 membered saturated ring system optionally having in addition to the N one or two heteroatoms selected from N—R₁, O, and S(═O)ₙ, where n is 0-2.

25. A compound according to claim 1, wherein A or B is a tricyclic heteroaryl group of the formula 3-A, 3-B, 4-A, 4-B, 5-A, 5-B, or 5-C 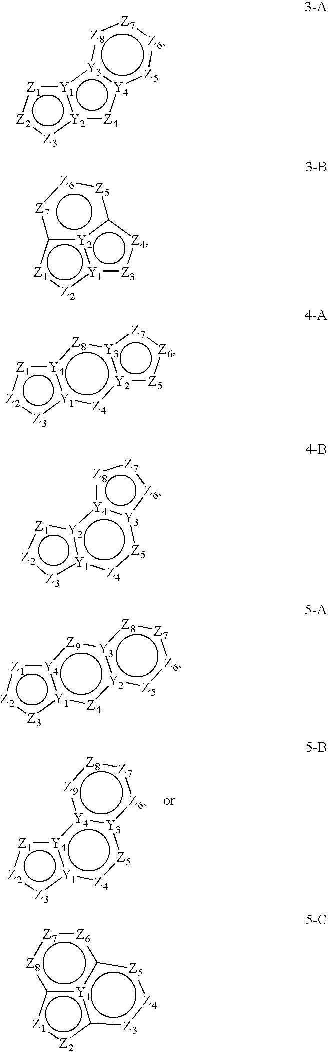
wherein:
one of Z₁, Z₂, Z₃, Z₄, Z₅, Z₆, Z₇, and Z₈ is a carbon bonded to the remainder of the molecule and the other Z₁, Z₂, Z₃, Z₄, Z₅, Z₆, Z₇, and Z₈ are independently selected from CR₂, N, O, S, and N—R₁; and Y₁, Y₂, Y₃, Y₄, R₁, and R₂ are as defined in claim 24.

26. A compound according to claim 1, wherein A or B is a tricyclic heteroaryl group of the formula 6-A or 6-B 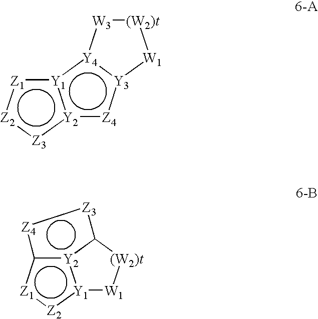
wherein:
one of Z₁, Z₂, Z₃, and Z₄, is a carbon bonded to the remainder of the molecule and the other Z₁, Z₂, Z₃, and Z₄ are independently selected from CR₂, N, O, S, and N—R₁;
W₁, W₂, and W₃ are each independently selected from CR₄R₄, S(═O)r where r is 0-2, O, and N—R₁, with the proviso that whenever W₂ is S or O it is not adjacent to a W₁, W₂ or W₃ that is also S or O each R₄ is independently H or C₁₋₆alkyl optionally substituted with nitro, C₆₋₁₄aryl, a 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl, C₁₋₆alkoxy, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, alkoxyalkyl-, C₁₋₆alkyl-S—, —SO₂N—R₆R₇, C₆₋₁₄aryl-O—, heteroaryl-O—, —S((═O)ₛ-aryl where s is 0-2, -alkyl-O-alkyl-NR₆R₇, -alkyl-aryl-O-alkylN—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; or any one of R₄ can be OH, C₁₋₆alkoxy, COOR₆, —NR₆R₇, —CONR₆R₇; or R₄R₄ may together be (═O); or R₄R₄ together with the carbon to which they are attached may form a spiro ring of five to eight members optionally having 1-3 heteroatoms selected from N, O, S(═O)ₙ where n is 0-2, and N—R₁;
t is 1-3; and
Y₁, Y₂, Y₃, Y₄, R₁, R₂, R₆, and R₇ are as defined in claim 24.

27. A compound according to claim 1, wherein A or B is a tricyclic heteroaryl group of the formula 7-A, 7-B, or 7-C 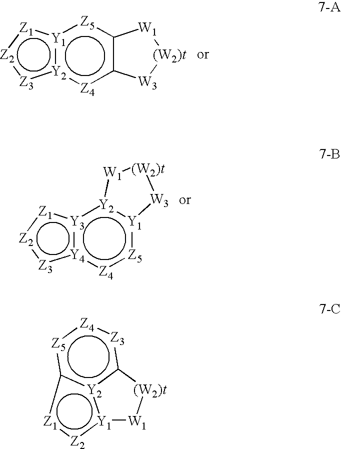
wherein:
one of Z₁, Z₂, Z₃, Z₄, and Z₅, is a carbon bonded to the remainder of the molecule and the other Z₁, Z₂, Z₃, Z₄ and Z₅ are independently selected from CR₂, N, O, S and N—R₁;
t is 1-3; and
Y₁, Y₂, Y₃, Y₄, W₁, W₂, W₃, R₁, R₂, R₄, R₆, and R₇ are as defined in claim 24.

28. A compound according to claim 1, wherein A or B is a tricyclic heteroaryl group of the formula 8-A or 8-B 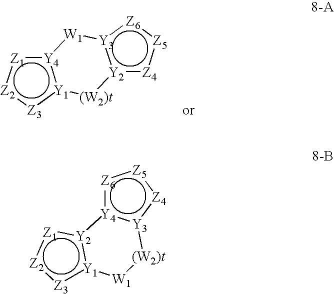
wherein:
one of Z₁, Z₂, Z₃, Z₄, Z₅, and Z₆ is a carbon bonded to the remainder of the molecule and the other Z₁, Z₂, Z₃, Z₄, Z₅ and Z₆ are independently selected from CR₂, N, O, S, and N—R₁;
t is 1-3; and
Y₁, Y₂, Y₃, Y₄, W₁, W₂, R₁, R₂, R₄, R₆, and R₇ are as defined in claim 24.

29. A compound according to claim 1, wherein A or B is a tricyclic heteroaryl group of the formula 9-A or 9-B 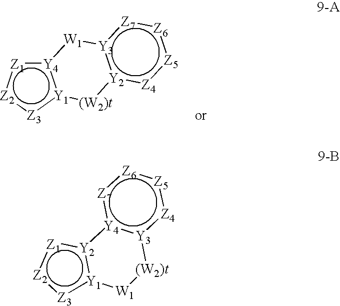
wherein:
one of Z₁, Z₂, Z₃, Z₄, Z₅, Z₆, and Z₇ is a carbon bonded to the remainder of the molecule and the other Z₁, Z₂, Z₃, Z₄, Z₅, Z₆ and Z₇ are independently selected from CR₂, N, O, S, and N—R₁;
t is 0-3; and
Y₁, Y₂, Y₃, Y₄, W₁, W₂, R₁, R₂, R₄, R6, and R₇ are as defined in claim 24.

30. A compound according to claim 1 or 20, wherein A or B is a tricyclic heteroaryl group of the formula 10-A or 10-B 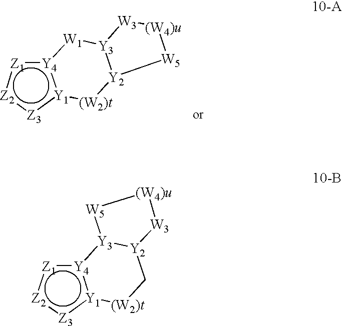
wherein:
one of Z₁, Z₂, and Z₃ is a carbon bonded to the remainder of the molecule and the other Z₁, Z₂ and Z₃ are independently selected from CR₂, N, O, S, and N—R₁;
Y₁ and Y₄ are independently C or N; Y₂ and Y₃ are independently CH or N; W₁, W₂, W₃, W₄, and W₅ are independently selected from CR₄R₄, S(═O)r where r is 0-2, 0, and N—R₁; with the proviso that whenever W₂ is S or O it is not adjacent to a W₁, W₂ or W₃ that is also S or O, with the proviso that whenever W₂ is S or O it is not adjacent to a W1, W₂ or W3 that is also S or O; t is 0-2; u is 1-3; and
R₁, R₂ and R₄ are as defined in claim 24.

31. A compound according to claim 1, wherein A or B is a tricyclic heteroaryl group of the formula 11-A or 11-B 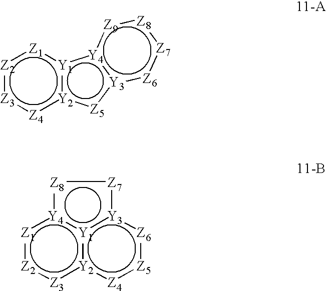
wherein
one of Z₁, Z₂, Z₃, Z₄, Z₅, Z₇, Z₈ and Z₉ is a carbon bonded to the remainder of the molecule and the other Z₁, Z₂, Z₃, Z₄, Z₅, Z₆, Z₇, Z₈ and Z₉ are independently selected from CR₂, N, O, S, and N—R₁; and
Y₁, Y₂, Y₃, Y₄, R₁ and R₂ are as defined in claim 24.

32. A compound according to claim 1, wherein A or B is a tricyclic heteroaryl group of the formula 12-A, 12-B, or 12-C 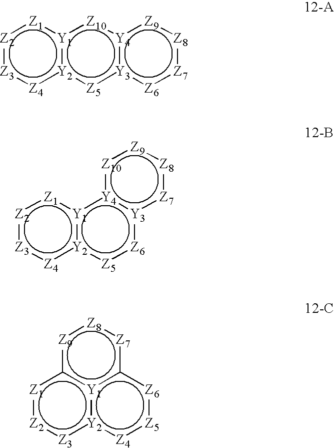
wherein:
one of Z₁, Z₂, Z₃, Z₄, Z₅, Z₆, Z₇, Z₈, Z₉ and Z₁₀ is a carbon bonded to the remainder of the molecule and the other Z₁Z₂Z₃, Z₄, Z₅, Z₆, Z₇, Z₈, Z₉, and Z₁₀ are independently selected from CR₂, N, O, S and N—R₁; Y₁, Y₂, Y₃, and Y₄ are independently C or N; and R₁ and R₂ are as defined in claim 24.

33. A compound according to claim 1, wherein the tricyclic heteroaryl group has the formula 13-A or 13-B 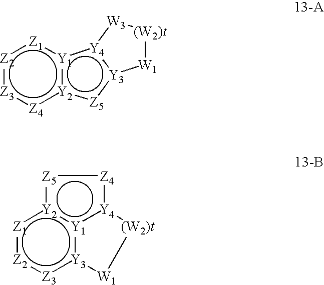
wherein:
one of Z₁, Z₂, Z₃, Z₄, and Z₅ is a carbon bonded to the remainder of the molecule and the other Z₁, Z₂, Z₃, Z₄ and Z₅ are independently selected from CR₂, N, O, S, and N—R₁;
t is 1-4; and
Y₁, Y₂, Y₃, Y₄, W₁, W₂, W₃, R₁, R₂, R₄, R₆, and R₇ are as defined in claim 24.

34. A compound according to claim 1, wherein A or B is a tricyclic heteroaryl group of the formula 14-A, 14-B, or 14-C 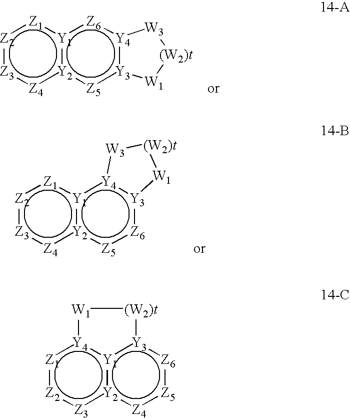
wherein:
one of Z₁, Z₂, Z₃, Z₄, Z₅, and Z₆ is a carbon bonded to the remainder of the molecule and the other Z₁, Z₂, Z₃, Z₄, Z₅, and Z₆ are independently selected from CR₂, N, O, S, and N—R₁;
t is 1-3; and
Y₁, Y₂, Y₃, Y₄, W₁, W₂, W₃, R₁, R₂, R₄, R₆, and R₇ are as defined in claim 24.

35. A compound according to claim 1, wherein A or B is a tricyclic heteroaryl group of the formula 15-A, 15-B, or 15-C 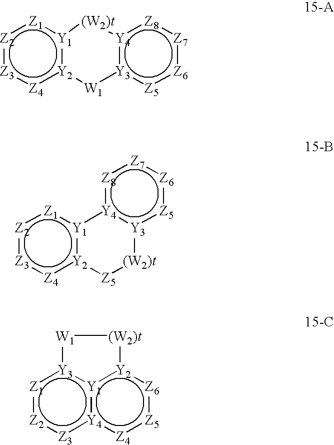
wherein:
one of Z₁, Z₂, Z₃, Z₄, Z₅, Z₆, Z₇, and Z₅ is a carbon bonded to the remainder of the molecule and the other Z₁, Z₂, Z₃, Z₄, Z₅, Z₆, Z₇ and Z₈ are independently selected from CR₂, N, O, S, and N—R₁;
t is 1-2; and
Y₁, Y₂, Y₃, Y₄, W₁, W₂, R₁, R₂, R₄, R₆, and R₇ are as defined in claim 24.

36. A compound according to claim 1 or 20 wherein the tricyclic heteroaryl group has the formula 16-A, 16-B, or 16-C 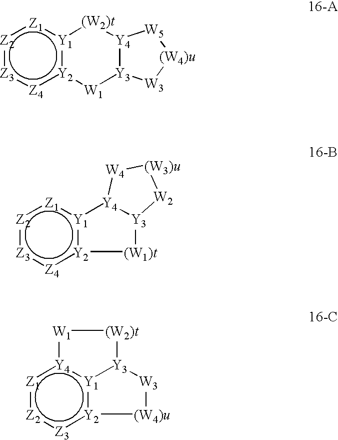
wherein:
one of Z₁, Z₂, Z₃, and Z₄ is a carbon bonded to the remainder of the molecule and the other Z₁, Z₂, Z₃, and Z₄ are independently selected from CR₂, N, O, S, and N—R₁;
t is 1-3;
u is 1-3; and
Y₁, Y₂, Y₃, Y₄, W₄, W₅, R₁, R₂, R₄, R₆, and R₇ are as defined in claim 28.

37. A compound of claim 1, wherein the compound is selected from:
(a) (5R), (6E)-6-(2,3-dihydro-imidazo[2,1-b]thiazol-5-ylmethylene)-7-oxo-1-aza-bicyclo[3.2.0]hept-2-ene-2-carboxylic acid, sodium salt;
(b) (5R), (6E)-6-[(5-benzyl-4,5,6,7-tetrahydrothieno[3,2-c]pyridin-2-yl)methylene]-7oxo-1- azabicyclo[3.2.0]hept-2-ene-2-carboxylic acid, sodium salt; or
(c) (5R), (6E)-6-(7-methyl-5,6,7,8-tetrahydroimidazo[1,2-a]pyrazin-2-ylmethylene)-7-oxo-1-aza-bicyclo[3.2.0]hept-2-ene-2-carboxylic acid, sodium salt; or
(d) (5R), (6E)-7-oxo-6-(5,6,7,8-tetrahydroimidazo[1,2-a]pyrazin-2-ylmethylene)-1-azabicyclo[3.2.0]hept-2-ene-2-carboxylic acid, sodium salt; or
(e) (5R), (6E)-6-{[5-(4-methoxybenzyl)-4,5,6,7-tetrahydrothieno[3,2-c]pyridin-2-yl)]methylene}-7-oxo-1-azabicyclo[3.2.0]hept-2-ene-2-carboxylic acid, sodium salt; or
(f) (5R), (6E)-6-(5,6-dihydro-8H-imidazo[2,1-c][1,4]thiazin-2-ylmethylene)-7-oxo-1-azabicyclo[3.2.0]hept-2-ene-2-carboxylic acid, sodium salt; or
(g) (5R), (6E)-6-(6,7-dihydro-5H-pyrrolo[1,2-a]imidazol-2-ylmethylene)-7-oxo-1-aza-bicyclo[3.2.0]hept-2-ene-2-carboxylic acid, sodium salt; or
(h) (5R), (6E)-6-(5,6-dihydro-8H-imidazo[2,1-c][1,4]oxazin-2-ylmethylene)-7-oxo-1-azabicyclo[3.2.0]hept-2-ene-2-carboxylic acid, sodium salt; or
(i) (5R), (6E)-6-(5,6-dihydro-4H-pyrrolo[1,2-b]pyrazol-2-ylmethylene)-7-oxo-1-azabicyclo[3.2.0]hept-2-ene-2-carboxylic acid, sodium salt; or
(j) (5R), (6E)-7-oxo-6-(4,5,6,7-tetrahydropyrazolo[1,5-a]pyridin-2-ylmethylene)-1- azabicyclo[3.2.0]hept-2-ene-2-carboxylic acid, sodium salt; or
(k) (5R), (6E)-6-(7-methyl-6-oxo-5,6,7,8-tetrahydro-imidazo[1,2-a]pyrazin-2-ylmethylene)-7-oxo-1-aza-bicyclo[3.2.0]hept-2-ene-2-carboxylic acid sodium salt; or
(l) (5R), (6E)-6-(6,7-dihydro-4H-pyrazolo[5,1-c][1,4]thiazin-2-ylmethylene)-7-oxo-1-azabicyclo[3.2.0]hept-2-ene-2-carboxylic acid, sodium salt; or
(m) (5R), (6E)-7-Oxo-6-(4H-5-thia-1,6a-diazapentalen-2-ylmethylene)-1-azabicyclo[3.2.0]hept-2-ene-2-carboxylic acid, sodium salt; or
(n) (5R), (6E)-6-(7H-imidazo[1,2-c]thiazol-2-ylmethylene)-7-oxo-1-azabicyclo[3.2.0]hept-2-ene-2-carboxylic acid, sodium salt; or
(o) (5R), (6E)-7-oxo-6-[(4-oxo-6,7-dihydro-4H-pyrazolo[5,1-c][1,4]oxazin-2-yl)methylene]-1-azabicyclo[3.2.0]hept-2-ene-2-carboxylic acid; or
(p) 6-(6,7-dihydro-4H-thieno[3,2-c]pyran-2-ylmethylene)-7-oxo-1-aza-bicyclo[3.2.0]hept-2-ene-2-carboxylic acid; or
(q) 6-(6,7-dihydro-4H-thieno[3,2-c]thiopyran-2-ylmethylene)-7-oxo-1-aza-bicyclo[3.2.0]hept-2-ene-2-carboxylic acid; or
(r) 6-(5-methyl-4,5,6,7-tetrahydro-thieno[3,2-c]pyridin-2-ylmethylene)-7-oxo-1-aza-bicyclo[3.2.0]hept-2-ene-2-carboxylic acid; or
(s) 2-(2-Carboxy-7-oxo-1-aza-bicyclo[3.2.0]hept-2-en-6-ylidenemethyl)-6,7-dihydro-4H-thieno[3,2-c]pyridine-5-carboxylic acid ethyl ester; or
(t) 7-oxo-6-(6,7,8,9-tetrahydro-5H-imidazo[1,2-a]azepin-2-ylmethylene)-1-aza-bicyclo[3.2.0]hept-2-ene-2-carboxylic acid; or
(u) (5R), (6E)-6-(7-benzyl-5,6,7,8-tetrahydroimidazo[1,2-a]pyrazin-2-ylmethylene)-7-oxo-1-azabicyclo[3.2.0]hept-2-ene-2-carboxylic acid; or
(v) (5R), (6E)-7-oxo-6-{[5-(pyridin-3-ylmethyl)-4,5,6,7-tetrahydrothieno[3,2-c]pyridin-2-yl)]methylene}-7oxo-1-azabicyclo[3.2.0]hept-2-ene-2-carboxylic acid; or
(w) (5R), (6E)-7-oxo-6-{[5-(pyridin-3-ylcarbonyl)-4,5,6,7-tetrahydrothieno[3,2-c]pyridin-2-yl]methylene}-7oxo-1-azabicyclo[3.2.0]hept-2-ene-2-carboxylic acid; or
(x) (5R), (6E)-7-oxo-6-{[5-(phenylacetyl)-4,5,6,7-tetrahydrothieno[3,2-c]pyridin-2-yl]methylene}-7oxo-1-azabicyclo[3.2.0]hept-2-ene-2-carboxylic acid; or
(y) (5R), (6E)-6-(6,7-dihydro-5H-cyclopenta[d]imidazo[2,1-b][1,3]thiazol-2-ylmethylene)-7-oxo-1-azabicyclo[3.2.0]hept-2-ene-2-carboxylic acid; or
(z) (5R), (6E)-7-oxo-6-(5,6,7,8-tetrahydroimidazo[2,1-b][1,3]benzothiazol-2-ylmethylene)-1-azabicyclo[3.2.0]hept-2-ene-2-carboxylic acid sodium salt; or
(aa) (5R), (6E)-6-(5,8-dihydro-6H-imidazo[2,1-b]pyrano[4,3-d][1,3]thiazol-2-ylmethylene)-7-oxo-1- azabicyclo[3.2.0]hept-2-ene-2-carboxylic acid; or
(bb) (5R), (6E)-6-(4,5,6,7-tetrahydro-1,3a,3b,8-tetraaza-cyclopenta[a]indene-2-ylmethylene)-7-oxo-1-aza-bicyclo[3.2.0]hept-2-ene-2-carboxylic acid sodium salt;
(cc) (5R), (6E)-6-(5H-imidazo[2,1-a]isoindol-2-ylmethylene)-7-oxo-1-aza-bicyclo[3.2.0]hept-2-ene-2-carboxylic acid sodium salt; or
(dd) (5R), (6E)-7-oxo-6-(5,6,7,8-tetrahydropyrazolo[5,1-b][1,3]benzoxazol-2-ylmethylene)-1-azabicyclo[3.2.0]hept-2-ene-2-carboxylic acid, sodium salt; or
(ee) (5R), (6E)-6-(7,8-dihydro-5H-pyrano[4,3-d]pyrazolo[5,1-b][1,3]oxazol-2-ylmethylene)7-oxo-1-azabicyclo[3.2.0]hept-2-ene-2-carboxylic acid, sodium salt; or
(ff) (5R), (6E)-6-{[6-(ethoxycarbonyI)-5,6,7,8-tetrahydropyrazolo[5′,1′,2,3][1,3]oxazolo[5,4-c]pyridin-2-yl]methylene}-7-oxo-1-azabicyclo[3.2.0]hept-2-ene-2-carboxylic acid, sodium salt; or
(gg) (5R), (6E)-6-({5-[2-(benzyloxy)ethoxy]-7,8-dihydro-6H-cyclopenta[e]imidazo[1,2-a]pyrimidin-2-yl}methylene)-7-oxo-1-azabicyclo[3.2.0]hept-2-ene-2-carboxylic acid, sodium salt; or
(hh) (5R), (6E)-6-[(5-methoxy-7,8-dihydro-6H-cyclopenta[e]imidazo[1,2-a]pyrimidin-2-yl)methylene]-7-oxo-1-azabicyclo[3.2.0]hept-2-ene-2-carboxylic acid, sodium salt; or
(ii) (5R), (6E)-6-imidazo[1,2-a]quinolin-2-ylmethylene-7-oxo-1-azabicyclo[3.2.0]hept-2-ene-2-carboxylic acid; or
(jj) (5R), (6E)-6-(imidazo[1.2-a]quinoxaline-2-ylmethylene)-7-oxo-1-azabicyclo[3.2.0]hepto-2-ene-2-carboxylic acid, sodium salt; or
(kk) (5R), (6E)-6-(imidazo[2,1-b]benzothiazol-7-ylmethylene)-7-oxo-1-azabicyclo[3.2.0]hept-2-ene-2-carboxylic acid; or
(ll) (5R), (6E)-6-(2,3-dihydro[1,3]thiazolo[3,2-a]benzimidazol-6-ylmethylene)-7-oxo-1-azabicyclo[3.2.0]hept-2-ene-2-carboxylic acid, sodium salt; or
(mm)(5R), (6E)-7-oxo-6-([1,3]thiazolo[3,2-a]benzimidazol-6-ylmethylene)-1-azabicyclo[3.2.0]hept-2-ene-2-carboxylic acid, sodium salt; or
(nn) (5R), (6E)-6-(3,4-dihydro-2H-[1,3]thiazino[3,2-a]benzimidazol-7-ylmethylene)-7-oxo-1-azabicyclo[3.2.0]hept-2-ene-2-carboxylic acid, sodium salt; or
(oo) (5R), (6E)-7-oxo-6-([1,3]thiazolo[3,2-a]benzimidazol-2-ylmethylene)-1-1-azabicyclo[3.2.0]hept-2-ene-2-carboxylic acid; or
(pp) (5R), (6E)-7-oxo-6-(4H-thieno[2′,3′,4,5]thiopyrano[2,3-b]pyridin-2-ylmethylene)-1-azabicyclo[3.2,0]hept-2-ene-2-carboxylic acid, sodium salt; or
(qq) (5R), (6E)-8-[(9-methyl-9H-imidazo[1,2-a]benzimidazol-2-yl)methylene]-7-oxo-1-azabicyclo[3.2.0]hept-2-ene-2-carboxylic acid sodium salt; or
(rr) (5R), (6E)-6-(7,8-dihydro-5H-pyrano[4,3-d]pyrazolo[5,1-b][1,3]oxazol-2-ylmethylene)7-oxo-1-azabicyclo[3.2.0]hept-2-ene-2-carboxylic acid, sodium salt; or
(ss) 6-(5-ethoxy-7,8-dihydro-6H-3,4,8b-triaza-as-indacen-2-ylmethylene)-7-oxo -1-aza-bicyclo[3.2.0]hept-2-ene-2-carboxylic acid; or
(tt) (5R), (6E)-6-(7,8-dihydro-5H-pyrano[4,3-d]pyrazolo[5,1-b][1,3]oxazol-2-ylmethylene)7-oxo-1-azabicyclo[3.2.0]hept-2-ene-2-carboxylic acid, sodium salt and (5R), (6E)-6-(7,8-dihydro-5H-pyrano[4,3-d]pyrazolo[5,1-b][1,3]oxazol-2-ylmethylene)7-oxo-1-azabicyclo[3.2.0]hept-2-ene-2-carboxylic acid, sodium salt; or
(uu) (5R), (6E)-6-{[6-(ethoxycarbonyl)-5,6,7,8-tetrahydropyrazolo [5′,1′:2, 3][1, 3}oxazolo [5,4-c]pyridin-2-yl]methylene}-7-oxo-1-azabicyclo[3.2.0]hept-2-ene-2-carboxylic acid, sodium salt.

38. A compound of claim 37, wherein the compound is (5R), (6E)-6-(2,3-dihydro-imidazo[2,1-b]thiazol-5-ylmethylene)-7-oxo-1-aza-bicyclo[3.2.0]hept-2-ene-2-carboxylic acid, sodium salt.

39. A method for treating a bacterial infection or disease comprising providing to a patient in need thereof an effective amount of cefepime or a pharmaceutically acceptable salt thereof and compound of formula I 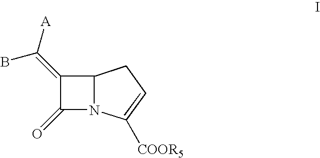
wherein:
one of A and B denotes hydrogen and the other an 8- to 14-membered fused bicyclic or tricyclic heteroaryl group optionally substituted with nitro, C₆₋₁₄aryl, -heteroaryl, C₁₋₆alkoxycarbonyl-, C₁₋₆alkoxy, -alkoxyalkyl, alkyl-O—C₂₋₄alkyl-O—, -cyano, -halogen, -hydroxy, -N—R₆R₇, -trifluoromethyl, -trifluoromethoxy, arylalkyl, alkylaryl, R₆R₇N-alkyl-, HO—C₁₋₆alkyl-, alkoxyalkyl-, alkyl-S—, —SO₂N—R₆R₇, —SO₂NHR₆, —CO₂H, CONR₆R₇, aryl-O—, heteroaryl-O—, —S(═O)ₛ-aryl where s is 0-2, -alkyl-O-alkyl-NR₆R₇, -alkyl-aryl-O-alkylN—R₆R₇, C₁₋₆alkyl, Cₘalkenyl, C₂₋₆alkynyl, C₃₋₇cycloalkyl, alkoxy-alkyl-O—, R₆R₇N—(C₁₋₆alkyl), and —S(═O)ₛ-heteroaryl where s is 0-2;
R₅ is H, C₁₋₆alkyl, C₅₋₆cycloalkyl, or CHR₃OCOC₁₋₆alkyl;
R₃ is hydrogen, C₁₋₆alkyl, C₃₋₇cycloalkyl, C₆₋₁₄aryl, or 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, wherein aryl or heteroaryl can be optionally substituted with 1 or 2 of nitro, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl, C₁₋₆alkoxy, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, alkyl-S—, —SO₂N—R₆R₇, aryl-O—, heteroaryl-O—, —S((═O)ₛ-aryl where s is 0-2, -alkyl-O-alkyl-NR₆R₇, -alkyl-aryl-O-alkylN-R₆R₇, -alkyl-aryl-O-alkylN—R₅R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; and
R₆ and R₇ are independently H, or a group selected from C₁₋₆alkyl, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, alkylaryl, arylalkyl, heteroarylalkyl, C₁₋₆alkylheteroaryl, said group being optionally substituted with nitro, C₆₋₁₄aryl, 5- to 10-membered heteroaryl having 1-3 heteroatoms independently selected from O, N, or S, a 5- to 10-membered mono or bicyclic saturated heterocyclyl having 1 to 3 heteroatoms selected from O, N, or S, C₁₋₆alkoxycarbonyl-, C₁₋₆alkoxy, -alkoxyalkyl, alkyl-O—C₂₋₄alkyl-O-cyano, -halogen, -hydroxy, —NR₆R₇, —COON, —COO-alkyl, -trifluoromethyl, -trifluoromethoxy, arylalkyl, alkylaryl, R₆R₇N-alkyl-, HO—C₁₋₆alkyl, alkoxyalkyl-, C₁₋₆alkyl-S—, —SO₂NHR₆, —CO₂H, CONR₆R₇, C₆₋₁₄aryl-O—, heteroaryl-O—, —S(═O)ₛ-aryl, wherein s is 0-2, -alkyl-NR₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)₅-heteroaryl, wherein S is 0-2, or R₆ and R₇ can together with the nitrogen to which they are attached form a 3 to 7 membered saturated ring system optionally having one or two heteroatoms selected from N—R₁, O, and S(═O)ₙ, where n is 0-2;
or a pharmaceutically acceptable salt thereof.

40. The method of claim 39, wherein A or B is a bicyclic heteroaryl group of the formula II

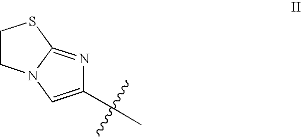

41. The method of claim 39, comprising co-administering cefepime or a pharmaceutically acceptable salt thereof and the compound of formula I or pharmaceutically acceptable salt or in vivo hydrolysable ester thereof.

42. The method of claim 41, wherein the ratio of cefepime or a pharmaceutically acceptable salt thereof to the compound of formula I or pharmaceutically acceptable salt or in vivo hydrolysable ester thereof is from about 1:1 to about 100:1.

43. The method of claim 42, wherein the ratio of cefepime or a pharmaceutically acceptable salt thereof to the compound of formula I or pharmaceutically acceptable salt or in vivo hydrolysable ester thereof is less than about 10:1.

44. The method of any one of claims 39-43, comprising orally administering to a patient.

45. The method of any one of claims 39-43, comprising intravenously administering to a patient.

46. A composition comprising a pharmaceutically acceptable carrier, cefepime or a pharmaceutically acceptable salt thereof, and a compound of formula I 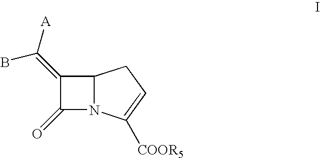
wherein:
one of A and B denotes hydrogen and the other an 8- to 14-membered fused bicyclic or tricyclic heteroaryl group optionally substituted with nitro, -aryl, -heteroaryl, C₁₋₆alkoxycarbonyl-, C₁₋₆alkoxy, -alkoxyalkyl, alkyl-O-C₂₋₄alkyl-O—, -cyano, -halogen, -hydroxy, -N—R₆R₇, -trifluoromethyl, -trifluoromethoxy, arylalkyl, alkylaryl, R₆R₇N-alkyl-, HO—C₁₋₆alkyl-, alkoxyalkyl-, alkyl-S—, —SO₂N—R₆R₇, —SO₂NHR₆, —CO₂H, CONR₆R₇, aryl-O—, heteroaryl-O—, —S(═O)ₛ-aryl where s is 0-2, -alkyl-O-alkyl-NR₆R₇, -alkyl-aryl-O-alkylN—R₆R₇, C₁₋₆alkyl, C₂₋₈alkenyl, C₂₋₆alkynyl, C₃₋₇cycloalkyl, alkoxy-alkyl-O—, R₆R₇N—(C₁₋₆alkyl), and -S(═O)ₛ-heteroaryl where s is 0-2;
R₅ is H, C₁₋₆alkyl, C₅₋₆cycloalkyl, or CHR₃OCOC₁₋₆alkyl; and
R₃ is hydrogen, C₁₋₆alkyl, C₃₋₇cycloalkyl, C₆₋₁₄aryl, or heteroaryl, wherein aryl or heteroaryl can be optionally substituted with 1 or 2 of nitro, -aryl, -heteroaryl, C₁₋₆alkoxycarbonyl, C₁₋₆alkoxy, -alkoxyalkyl, alkyl-O—C₂₋₄alkyl-O—, -cyano, -halogen, -hydroxy, —NR₆R₇, -trifluoromethyl, -trifluoromethoxy, arylalkyl, alkylaryl, R₆R₇N-alkyl-, HO—C₁₋₆alkyl-, alkoxyalkyl-, alkyl-S—, —SO₂N—R₆R₇, aryl-O—, heteroaryl-O—, —S((═O)ₛ-aryl where s is 0-2, -alkyl-O-alkyl-NR₆R₇, -alkyl-aryl-O-alkylN—R₆R₇, -aryl-O-alkylN—R₆R₇, C₁₋₆alkyl, C₂₋₈alkenyl, C₂₋₆alkynyl, C₃₋₇cycloalkyl, alkoxy-alkyl-O—, R₆R₇N-alkyl-, and —S(═O)ₛ-heteroaryl where s is 0-2; or a pharmaceutically acceptable salt thereof.

47. The composition of claim 46, wherein the bicyclic heteroaryl group has the formula II:

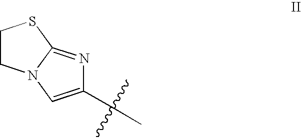

48. The composition of claim 46, wherein the ratio of cefepime or a pharmaceutically acceptable salt thereof to the compound of formula I or pharmaceutically acceptable salt or in vivo hydrolysable ester thereof is from about 1:1 to about 100:1.

49. The composition of claim 48, wherein the ratio of cefepime or a pharmaceutically acceptable salt thereof to the compound of formula I or pharmaceutically acceptable salt or in vivo hydrolysable ester thereof is less than about 10:1.

50. A process for preparing a compound of formula I as claimed in claim 1, which comprises subjecting to reductive elimination a compound of formula III: 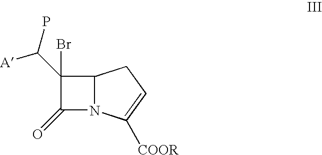
wherein A′ is A or B as defined in claim 1, P is an ester leaving group, R is a protecting group that can be removed to give a compound of formula I 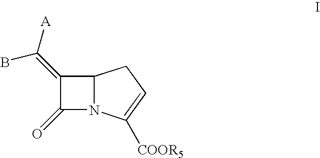
or a pharmaceutically acceptable salt or ester thereof.

51. The process according to claim 50, wherein the ester leaving group is selected from O-acetate, O-mesylate, O-triflate, O-tosylate, and tosylate.

52. The process according to claim 50 or 51, wherein the protecting group is a para-nitrobenzyl group.
